(19) United States
(12) Patent Application Publication
(10) Pub. No.: US 2021/0403415 A1
(43) Pub. Date: Dec. 30, 2021

(54) COMPOUNDS AND METHODS TO ATTENUATE TUMOR PROGRESSION AND METASTASIS

(71) Applicant: THE TRUSTEES OF INDIANA UNIVERSITY, Bloomington, IN (US)

(72) Inventors: Samy MEROUEH, Carmel, IN (US); Khuchtumur BUM-ERDENE, Indianapolis, IN (US)

(21) Appl. No.: 17/288,181

(22) PCT Filed: Oct. 28, 2019
(86) PCT No.: PCT/US2019/058294

§ 371 Date: Apr. 23, 2021

Related U.S. Application Data
(60) Provisional application No. 62/751,190, filed on Oct. 26, 2018.

(51) Int. Cl. C07C 229/60; C07C 225/22; C07D 213/74; C07D 333/20; C07D 495/04; C07C 233/43; C07C 233/44

(52) U.S. Cl. CPC C07C 229/60; C07C 225/22; C07D 213/74; C07C 233/44; C07D 495/04; C07C 233/43; C07D 333/20

(57) ABSTRACT

This invention relates to certain compounds or pharmaceutically acceptable salts thereof, and for the use of the compounds to treat cancer. In another aspect, the disclosure relates to a pharmaceutical composition comprising a compound of Formula (1), Formula (1a), or Formula (1b), or a pharmaceutically acceptable salt thereof, and at least one of a pharmaceutically acceptable carrier, diluent or excipient.

### CROSS-REFERENCE TO RELATED APPLICATIONS

This application claims the benefit under 35 U.S.C. § 119(e) of U.S. Provisional Application No. 62/751,190, filed on Oct. 26, 2018, the entire disclosure of which is incorporated herein by reference.

### SEQUENCE LISTING

The instant application contains a Sequence Listing which has been submitted electronically in ASCII format and is hereby incorporated by reference in its entirety. The ASCII copy, created on Oct. 23, 2019, is named 298907_ST25.txt and is 5 kilobytes.

### BACKGROUND

The Yes-associated protein (YAP) is a transcriptional coactivator. Yap needs to bind transcription factors to stimulate gene expression. Reported YAP target transcription factors include TEA/ATS (TEAD) transcription factors. In humans, the TEAD family has four highly homologous proteins sharing a conserved DNA-binding TEA domain.

TEAD and its YAP coactivator have a central role is many cancers. A need remains for compounds that inhibit TAP/TEAD interaction to treat cancer.

### SUMMARY

In one aspect, the disclosure relates to a compound of Formula (1a)

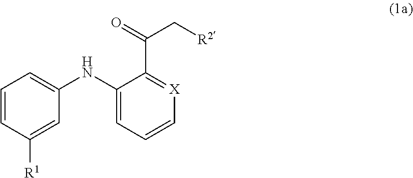

wherein

R¹ is C(R)₃ or lower (C₁-C₆) alkyl, where one or more of the carbon atoms in lower (C₁-C₆) alkyl are replaced by oxygen; where R is a halogen;

R^{2′} is halogen or lower (C₁-C₆) alkyl, where one or more of the hydrogens in lower (C₁-C₆) alkyl are replaced with a halogen; and

X is carbon or a heteroatom independently selected from nitrogen, oxygen and sulfur;

or a pharmaceutically acceptable salt thereof.

In another aspect, the disclosure relates to a compound of the of Formula (1)

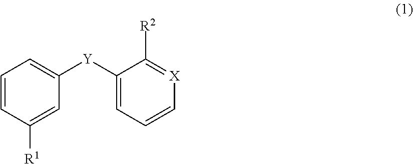

wherein

Y is selected from the group consisting of —C(O)—, —NH—, —O—, —S—, —CH₂—, and —S(O)₂—;

R¹ is C(R)₃ or lower (C₁-C₆) alkyl, where one or more of the carbon atoms in lower (C₁-C₆) alkyl are replaced by oxygen; where R is a halogen;

R² is selected from the group consisting of —CH₂CN, —CN, —NHC(O)CHCH₂, —S(O)₂CHCH₂, —NHC(O)CHCHCH₂N(CH₃)₂, —NHC(O)CCH, —C(O)CH₂R^{2′}, and —NHC(O)CH₂R^{2′}, where R^{2′} is halogen or lower (C₁-C₆) alkyl, where one or more the hydrogens in lower (C₁-C₆) alkyl are replaced with a halogen; and

X is carbon or a heteroatom independently selected from nitrogen, oxygen, and sulfur;

or a pharmaceutically acceptable salt thereof.

In another aspect, the disclosure relates to a compound of the of Formula (1b)

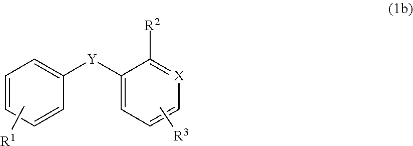

wherein

Y is selected from the group consisting of —C(O)—, —NH—, —O—, —S—, —CH₂—, and —S(O)₂;

R¹ is selected from the group consisting of —C(R)₃, thiophene, —O(CH₂)ₙ(OCH₂CH₂)ₘNH-biotin, and lower (C₁-C₆) alkyl, where one or more of the carbon atoms in lower (C₁-C₆) are replaced by oxygen, n is an integer from 1 to 6 and m is an integer from 1 to 3; where R is a halogen;

R² is selected from the group consisting of —CH₂CN, —CN, —NHC(O)CHCH₂, —S(O)₂CHCH₂, —NHC(O)CHCHCH₂N(CH₃)₂, —NHC(O)CCH, —C(O)CH₂R^{2′}, and —NHC(O)CH₂R^{2′}, where R^{2′} is halogen or lower (C₁-C₆) alkyl where one or more the hydrogens in lower (C₁-C₆) alkyl are replaced with a halogen;

R³ is H or C₁-C₆ alkoxy,

X is carbon or a heteroatom independently selected from nitrogen, oxygen and sulfur;

or a pharmaceutically acceptable salt thereof.

In another aspect, the disclosure relates to a pharmaceutical composition comprising a compound of Formula (1), Formula (1a), or Formula (1b), or a pharmaceutically acceptable salt thereof, and at least one of a pharmaceutically acceptable carrier, diluent or excipient.

In another aspect, the disclosure relates to a method to treat cancer in a mammal in need thereof, comprising administering an effective amount of a compound of Formula (1), Formula (1a), or Formula (1b).

In another aspect, the disclosure relates to a method to modulate a protein-protein interaction between a Yes-associated protein (YAP) transcriptional coactivator and a TEA/ATS (TEAD) transcription factor in a cell comprising contact the cell with a compound of Formula (1), Formula (1a), or Formula (1b).

Additional embodiments, features, and advantages of the disclosure will be apparent from the following detailed description and through practice of the disclosure.

### BRIEF DESCRIPTION OF THE DRAWINGS

FIGS. 1A-1B show an illustrative representation of a TEAD4/Yap1 complex, wherein FIG. 1A shows stereo views of the X-ray structure of the TEAD4/Yap1 complex and FIG. 1B shows the structure of the TEAD4/Yap1 complex and depicts the hydrophobic pocket of TEAD4 occupied by palmitate represented by the stick structure and letters PLM.

FIGS. 2A-2B show the bonds between compound 2 bound to TEAD4, wherein FIG. 2A shows the non-covalent bonds and FIG. 2B shows the covalent bonds.

FIGS. 3A-3H show graphs, wherein FIG. 3A is a graphic representation of increasing concentration of TEAD4 incubated with 16 nM FAM-labled Yap peptide; FIG. 3B shows the change in fluorescene polarization (FP) as TEAD4 is incubated with increasing concentration of compounds 1, 2, or 3; FIG. 3C shows time-dependent inhibition of TEAD4 by compound 2; FIG. 3D shows the observed rate of inactivation (k_{obs}) calculated at various compound 2 concentrations; FIG. 3E shows the results of incubating TEAD4 with 50 μM of compound 2; FIG. 3F shows ESI mass spectrometry analysis of 10 μM of TEAD4 incubated with 200 μM of DMSO or compound 2 or 3; FIG. 3G shows the percent ratio of the adduct over total protein signal; and FIG. 3H shows no significant aggregation was observed when TEAD4 was incubated with DMSO or compound 2.

FIGS. 4A-4I are graphs, wherein FIG. 4A shows an ESI mass spectrometry analysis of TEAD4 Cys367Ser mutant (10 μM) incubated with 200 μM of compound 2; FIG. 4B shows the measurement of FP of increased TEAD4 Cys367Ser mutant mixed with 16 nM FAM-Yap₆₀₋₉₉ peptide; FIG. 4C shows the percent of inhibition as TEAD4 Cys367Ser mutant was incubated with compound 1 or 2; FIG. 4D shows the interferometry sensor response when Yap1 interacted with various concentrations of TEAD4; FIG. 4E shows the percent relevant response to Yap1 interacting 100 nM of TEAD4 that was preincubated with varying concentrations of compound 2; FIG. 4F shows increased concentration of His-TEAD2 mixed with 16 nM FAM-Yap₆₀₋₉₉ peptide; FIG. 4G shows His-TEAD2 incubated with increasing concentration of compound 1, 2, or 3; FIG. 4H shows the percent inhibition of urokinase receptor incubated with varying concentrations of compound 2 or 3; and FIG. 4I shows the β-3 subunit of the voltage-gated calcium channel Cav2.2 was incubated with varying concentrations of compounds 2 or 3 followed by addition of an α-subunit peptide for FP measurement.

FIGS. 5A-5B are cartoon representations wherein FIG. 5A depicts a stereo image of compound 2 covalently bound to Cys-380 in the central binding pocket of TEAD2, and FIG. 5B depicts a two-dimensional ligand interaction map of covalently bound compound 2 in the central pocket of TEAD2.

FIG. 6A-6K are graphs, wherein FIG. 6A shows TEAD4 was incubated with increasing concentration of compounds 4, 5, 6, 7, and 8; FIG. 6B shows TEAD4 Cys367Ser mutant was incubated with increasing concentrations of compounds 4, 5, 6, 7, and 8; FIG. 6C shows 10 μM TEAD4 was incubated with 200 μM compounds 4, 5, 6, 7, and 8 and then analyzed by ESI mass spectrometry; FIG. 6D shows 10 μM TEAD4 Cys367Ser mutant was incubated with 200 μM compounds 4, 5, 6, 7, and 8 and then analyzed by ESI mass spectrometry; FIG. 6E shows His-TEAD2 was incubated with increasing concentration of compounds 4, 5, 6, 7, and 8 for FP measurement; FIG. 6F shows time-dependent inhibition of TEAD4 by compound 4; FIG. 6G shows time-dependent inhibition of TEAD4 by compound 5; FIG. 6H shows time-dependent inhibition of TEAD4 by compound 6; FIG. 6I shows time-dependent inhibition of TEAD4 by compound 4 and the observed rate of inactivation (k_{obs}) was calculated for each concentration of compound 4; FIG. 6J shows time-dependent inhibition of TEAD4 by compound 5 and the k_{obs} was calculated for each concentration of compound; and FIG. 6K shows time-dependent inhibition of TEAD4 by compound 6 and the k_{obs} was calculated for each concentration of compound 6.

FIGS. 7A-7F show that compound 2 inhibits TEAD transcriptional activity and protein-protein interaction in cell culture, wherein FIG. 7A shows the activity of the TEAD4 measured in HEK-293 cells treated with either vehicle or compound 2 wherein CNYT corresponds to no transfection; FIG. 7B shows co-immunoprecipitation of FLAG-tagged Yap1 and myc-tagged TEAD4 from lysates of HEK-293 cells treated with vehicle, compound 2, or a peptide (FAM-Yap₆₀₋₉₉); FIG. 7C shows average normalized values relative to a corresponding lane from FIG. 7B for three biologic replicates; FIG. 7D shows lysates from HEK-293 cells treated with compounds 2, 5, or 9; FIG. 7E shows average normalized values relative to a corresponding lane from FIG. 7D for three biological replicates; FIG. 7F shows qRT-PCR analysis of CTGF levels following treatment of HEK-293 cells with DMSO or compounds 2, 3, or 5.

FIGS. 8A-8D show compound 2 inhibits patient derived GBM43 glioblastoma, wherein FIG. 8A shows spheroids of glioblastoma treated with compound 1, 2, or 5; FIG. 8B shows the activity of the TEAD4 in GBM43 cells treated with either vehicle or compound 2 and wherein CNYT corresponds to no transfection; FIG. 8C shows qRT-PCR analysis of CTGF levels following treatment of GBM43 cells with DMSO or compounds 2, 3, or 5; FIG. 8D shows GBM43 cells treated with temozolomide.

FIGS. 9A-9E show the total ion count when TEAD4 Cys-367 is reacted with iodoacetamide, wherein FIG. 9A shows a sample of 5 μM TEAD4 was reacted with iodoacetamide for 30 min; FIG. 9B for 6 hr; FIG. 9C for 24 hr, wherein wild-type TEAD4 YBD construct showed an apparent MW of 25952, and the iodoacetamide adduct was +57; FIG. 9D shows total ion count for a sample of 5 μM TEAD4 Cys367Ser mutant reacted with iodoacetamide and wherein TEAD4 YBD Cys367Ser mutant construct showed an apparent MW of 25936; and FIG. 9E shows the percent inhibition when TEAD4 was incubated with increasing concentration of iodoacetamide.

FIGS. 10A-10C are graphs showing that compound 2 does not inhibit TEAD mutant transcriptional activity nor protein-protein interactions in cell culture, wherein FIG. 10A shows the activity of the TEAD4 in HEK-293 cells at 24 hr treated with either vehicle or compound 2; FIG. 10B shows the activity of the TEAD4 Cys367Ser measured in HEK-293 cells at 24 hr treated with either vehicle or compound 2; and FIG. 10C shows coomassie stained gel of the pull-down samples, and wherein the arrow identifies myc-TEAD4.

FIG. 11 shows the amount of absorbance of MTS by human astrocytes treated by either DMSO or compound 2 at the indicated concentration on the x-axis.

FIG. 12 shows compound 2 inhibits TEAD transcriptional activity in a concentration-dependent manner in HEK-293 cells at 24 hr treated with either DMSO or compound 2.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially available ACD/Name 2014 (ACD/Labs) or ChemBioDraw ultra 13.0 (Perkin Elmer).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Definitions

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, C₁-C₄, and C₁-C₃. Illustratively, such particularly limited length alkyl groups, including C₁-C₈, C₁-C₇, C₁-C₆, C₁-C₄, and C₁-C₃ and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, “hydroxy” or “hydroxyl” refers to an —OH group.

As used herein, “alkoxy” refers to both an —O-(alkyl). Representative examples include, but are not limited to, methoxy, ethoxy, propoxy, butoxy, and the like.

As used herein, “halo” or “halogen” refers to fluorine, chlorine, bromine, or iodine.

As used herein, “bond” refers to a covalent bond.

As used herein, “biotin” refers to

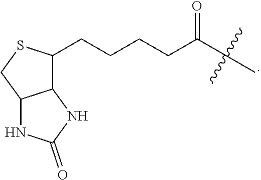 [SMILES: *C(=O)CCCCC1SCC2NC(=O)NC21]

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

As used herein, the phrase where one or more of the carbon atoms in lower (C₁-C₆) are replaced by oxygen means that at least one carbon in the C₁-C₆ chain has been replace with oxygen. For example, where one or more of the carbon atoms in pentyl are replaced by oxygen would lead to a structure such as —CH₂OCH₂CH₂CH₃, —CH₂CH₂CH₂OCH₃, —OCH₂CH₂OCH₃, and the like.

As used herein, the term “pharmaceutically acceptable salt” refers to those salts which counter ions which may be used in pharmaceuticals. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. Such salts include:

(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Pharmaceutically acceptable salts are well known to those skilled in the art, and any such pharmaceutically acceptable salt may be contemplated in connection with the embodiments described herein. Examples of pharmaceutically acceptable salts include sulfates, pyro sulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, is obutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenyl acetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (1), Formula (1a), or Formula (1b) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula (1), Formula (1a), or Formula (1b) and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (1), Formula (1a), or Formula (1b). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of compounds of Formula (1), Formula (1a), or Formula (1b), and uses of such metabolites in the methods of the disclosure. A “pharmaceutically active metabolite” means a pharmacologically active product of metabolism in the body of a compound of Formula (1) and Formula (1a), or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Although the present invention contemplates all individual enantiomers and diastereomers, as well as mixtures of the enantiomers of the compounds, including racemates, the compounds with the absolute configuration as set forth below are particularly preferred.

Individual isomers, enantiomers, and diastereomers may be separated or resolved by one of ordinary skill in the art at any convenient point in the synthesis of compounds of the invention, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Any disubstituent referred to herein is meant to encompass the various attachment possibilities when more than one of such possibilities are allowed. For example, reference to disubstituent A-B—, where A≠B, refers herein to such disubstituent with A attached to a first substituted member and B attached to a second substituted member, and it also refers to such disubstituent with A attached to the second substituted member and B attached to the first substituted member.

### Representative Embodiments

In some embodiments, compounds described herein comprise a compound of Formula (1a)

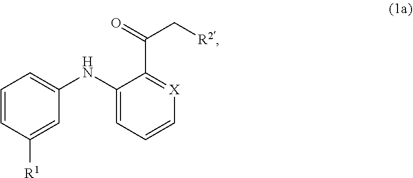

or a pharmaceutically acceptable salt thereof.

In some embodiments, compounds described herein comprise a compound of Formula (1)

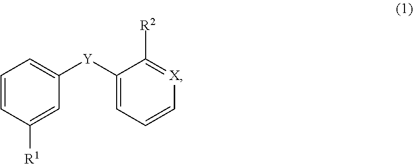

or a pharmaceutically acceptable salt thereof.

In some embodiments, compounds described herein comprise a compound of Formula (1b)

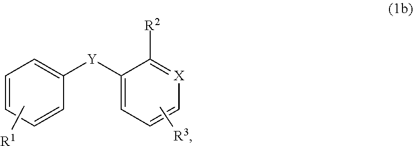

or a pharmaceutically acceptable salt thereof.

In some embodiments, R¹ is C(R)₃, where R is a halogen, or lower (C₁-C₆) alkyl. In some embodiments, R¹ is selected from the group consisting of —C(R)₃ where R is a halogen, thiophene, —O(CH₂)ₙ(OCH₂CH₂)ₘNH-biotin, and lower (C₁-C₆) alkyl. In some embodiments, one or more of the carbon atoms in lower (C₁-C₆) are replaced by oxygen. In some embodiments, lower (C₁-C₆) alkyl is methyl, ethyl, or n-propyl. In some embodiments, R¹ is C(R)₃ where R is a halogen. In some embodiments, R is chloro or fluoro. In some embodiments, R is fluoro. In some embodiments, R¹ is thiophene.

In some embodiments, n is an integer from 1 to 6. In some embodiments, n is 5.

In some embodiments, m is an integer from 1 to 3. In some embodiments, m is 2.

In some embodiments, is R² is selected from the group consisting of —CH₂CN, —CN, —NHC(O)CHCH₂, —S(O)₂CHCH₂, —NHC(O)CHCHCH₂N(CH₃)₂, —NHC(O)CCH, —C(O)CH₂R^{2′}, and —NHC(O)CH₂R^{2′}. In some embodiments, R² is C(O)CH₂CH₃. In some embodiments, R² is —COOH.

In some embodiments, R^{2′} is halogen or lower (C₁-C₆) alkyl. In some embodiments, one or more the hydrogens in lower (C₁-C₆) alkyl are replaced with a halogen. In some embodiments, lower (C₁-C₆) alkyl is methyl, ethyl, or n-propyl. In some embodiments R^{2′} is halogen. In some embodiments, R^{2′} is chloro.

In some embodiments, R² is —C(O)CH₂R^{2′}. In some embodiments R^{2′} is halogen. In some embodiments, R^{2′} is chloro.

In some embodiments, R³ is H or C₁-C₆ alkoxy. In some embodiments, R³ is H. In some embodiments, R³ is alkoxy. In some embodiments, R³ is methoxy.

In some embodiments, Y is selected from the group consisting of —C(O)—, —NH—, —O—, —S—, —CH₂—, and S(O)₂—. In some embodiments, Y is —NH—.

In some embodiments, X is carbon or a heteroatom. In some embodiments, the heteroatom is independently selected from nitrogen, oxygen, and sulfur. In some embodiments, X is carbon or nitrogen. In some embodiments, X is carbon. In some embodiments, X is nitrogen.

This invention contemplates all individual enantiomers and diasteromers, as well as mixtures of the enantiomers of the compounds, including racemates. Individual isomers, enantiomers, and diastereomers may be separated or resolved by one of ordinary skill in the art at any convenient point in the synthesis of compounds of the invention, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

The invention further provides a method to treat a cancer in a patient in need of such treatment, comprising administering to the patient an effective amount of a compound of Formula (1a), Formula (1), or Formula (1b), or a pharmaceutically acceptable salt thereof.

The invention further provides a compound of Formula (1), Formula (1a), or Formula (1b), or a pharmaceutically acceptable salt thereof, for the manufacture of a medicament for the treatment of cancer.

The invention further provides a pharmaceutical composition, comprising a compound of Formula (1), Formula (1a), or Formula (1b), or a pharmaceutically acceptable salt thereof, with one or more pharmaceutically acceptable carriers, diluents, or excipients. The invention further provides a process for preparing a pharmaceutical composition, comprising admixing a compound of Formula (1), Formula (1a), or Formula (1b), or a pharmaceutically acceptable salt thereof, with one or more pharmaceutically acceptable carriers, diluents, or excipients. This invention also encompasses novel intermediates and processes for the synthesis of the compounds of Formula 1.

In an embodiment, R′ is CF₃. In an embodiment, R² is chlorine. In an embodiment, X is carbon or nitrogen.

In an embodiment, the compound of Formula (1), Formula (1a), or Formula (1b) binds to the TEAD4 palmitate pocket. In another embodiment, the compound of Formula (1), Formula (1a), or Formula (1b) form a covalent bond with a conserved cysteine. In a further embodiment, the compound of Formula (1), Formula (1a), or Formula (1b) disrupts TEAD4.Yap1 protein-protein interaction.

In embodiments of the invention, the compounds of Formula (1), Formula (1a), or Formula (1b) inhibit Yap1 transcriptional activity, thereby suppressing tumor activity, suppress. In other embodiments, the compounds of Formula (1), Formula (1a), or Formula (1b) suppress or inhibit expression of connective tissue growth factor (CTGF). In yet another embodiment, the compounds of Formula 1 act as chemical probes to explore Hippo signaling in cancer as the compounds inhibit cell viability of patient-derived glioblastoma spheroids.

The present invention also provides a pharmaceutical composition comprising a compound of Formula (1), Formula (1a), or Formula (1b), or a pharmaceutically acceptable salt thereof, together with at least one of a pharmaceutically acceptable carrier, diluent or excipient.

The compounds of the invention are preferably formulated as pharmaceutical compositions administered by any route which makes the compound bioavailable, including oral and transdermal routes. Most preferably, such compositions are for oral administration. Such pharmaceutical compositions and processes for preparing same are well known in the art (See, e.g., Remington: The Science and Practice of Pharmacy, L. V. Allen, Editor, 22^{nd} Edition, Pharmaceutical Press, 2012). The compounds of Formula I, or pharmaceutically acceptable salts thereof are particularly useful in the treatment methods of the invention.

This invention also provides a method to treat a cancer such as cancer in a mammal in need thereof. The method comprises administering to the mammal in need of treatment an effective amount of a compound, or a pharmaceutically acceptable salt thereof of Formula (1), Formula (1a), or Formula (1b). Cancers that may be treated with compounds of Formula (1), Formula (1a), or Formula (1b) include, but are not limited to, solid tumors such as glioblastoma multiforme, cancers of the lung (non-small cell lung cancer), thyroid, skin, ovaries, colon, rectum, prostate, pancreas (pancreatic ductal adenocarcinoma), esophagus, liver and breast (triple negative breast cancer).

This invention further provides a method to modulate, suppress, or inhibit protein-protein interaction between a Yes-associated protein (YAP) transcriptional coactivator and a TEA/ATS (TEAD) transcription factor in a cell, comprising contacting the cell with an effective amount of the compound of Formula (1), Formula (1a), or Formula (1b) or a pharmaceutically acceptable salt thereof. In one embodiment, the TEA/ATS (TEAD) transcription factor is selected from the group consisting of TEAD1, TEAD2, TEAD3, and TEAD4. In another embodiment, the YAP transcriptional coactivator is YAP1. In another embodiment, the cell is a mammalian cell. In a further embodiment, the cell is a cancer cell. In another embodiment, the compound of Formula (1), Formula (1a), or Formula (1b) forms a covalent bond or complex with cysteine residue Cys-367 in a central pocket in TEAD4. In one embodiment, the compound of Formula (1), Formula (1a), or Formula (1b) inhibits protein-protein interaction between a Yes-associated protein (YAP) transcriptional coactivator and a TEA/ATS (TEAD) transcription factor in a cell. In another embodiment, the compound of Formula (1), Formula (1a), or Formula (1b) irreversibly inhibits protein-protein interaction between a Yes-associated protein (YAP) transcriptional coactivator and a TEA/ATS (TEAD) transcription factor in a cell.

This invention also provides a process or method for preparing a compound of Formula (1), Formula (1a), or Formula (1b).

This invention further provides a method to identify a compound capable of forming an adduct with a conserved cysteine in a protein, the method, comprising identifying a protein having a conserved cysteine in an allosteric pocket and performing solvent molecular dynamics simulations on a compound.

A compound of the present invention can be provided as a pharmaceutically acceptable salt. A pharmaceutically acceptable salt of the compounds of the invention can be formed, for example, by reaction of an appropriate free base of a compound of the invention and an appropriate pharmaceutically acceptable acid in a suitable solvent under standard conditions well known in the art. The formation of such salts is well known and appreciated in the art. See, for example, Gould, P. L., “Salt selection for basic drugs,” International Journal of Pharmaceutics, 33: 201-217 (1986); Bastin, R. J., et al. “Salt Selection and Optimization Procedures for Pharmaceutical New Chemical Entities,” Organic Process Research and Development, 4: 427-435 (2000); and Berge, S. M., et al., “Pharmaceutical Salts,” Journal of Pharmaceutical Sciences, 66: 1-19, (1977).

The terms “treating” or “to treat” include restraining, slowing, stopping, or reversing the progression or severity of an existing symptom or disorder. As used herein, the term “patient” refers to a human. The term “effective amount” refers to the amount or dose of compound of the invention, or a pharmaceutically acceptable salt thereof which, upon single or multiple dose administration to the patient, provides the desired effect in the patient under diagnosis or treatment.

An effective amount can be readily determined by one skilled in the art by the use of known techniques and by observing results obtained under analogous circumstances. In determining the effective amount for a patient, a number of factors are considered, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compounds of Formula (1), Formula (1a), or Formula (1b) are generally effective over a wide dosage range. For example, dosages per day normally fall within the range of about 0.1 mg/kg to about 1.0 mg/kg of body weight. In some instances dosage levels below the lower limit of the aforesaid range may be more than adequate, while in other cases still larger doses may be employed with acceptable side effects, and therefore the above dosage range is not intended to limit the scope of the invention in any way.

The compounds of the present invention, or salts thereof, may be prepared by a variety of procedures known to one of ordinary skill in the art, some of which are illustrated in the schemes, preparations, and examples below. One of ordinary skill in the art recognizes that the specific synthetic steps for each of the routes described may be combined in different ways, or in conjunction with steps from different schemes, to prepare compounds of the invention, or salts thereof. The products of each step in the schemes below can be recovered by conventional methods well known in the art, including extraction, evaporation, precipitation, chromatography, filtration, trituration, and crystallization. In the schemes below, all substituents unless otherwise indicated, are as previously defined. The reagents and starting materials are readily available to one of ordinary skill in the art. Others may be made by standard techniques of organic and heterocyclic chemistry which are analogous to the syntheses of known structurally-similar compounds and the procedures described herein which follow including any novel procedures. Without limiting the scope of the invention, the following schemes, preparations, and examples are provided to further illustrate the invention. In addition, one of ordinary skill in the art appreciates that the compounds of Formula 1 may be prepared by using starting material with the corresponding stereochemical configuration which can be prepared by one of skill in the art.

The following represent illustrative embodiments of compounds of the Formula (1), Formula (1a), or Formula (1b):

| Compound | Structure | Name |
|---|---|---|
| 1 | 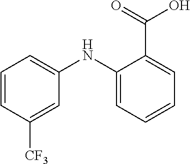 [SMILES: O=C(O)c1ccccc1Nc1cccc(C(F)(F)F)c1] | 2-((3-(trifluoromethyl)phenyl)amino)benzoic acid |
| 2 | 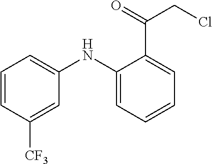 [SMILES: O=C(CCl)c1ccccc1Nc1cccc(C(F)(F)F)c1] | 2-chloro-1-(2-((3- (trifluoromethyl)phenyl)amino)phenyl) ethanone |
| 3 | 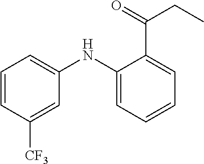 [SMILES: CCC(=O)c1ccccc1Nc1cccc(C(F)(F)F)c1] | 1-(2-((3- (trifluoromethyl)phenyl)amino)phenyl)propan- 1-one |
| 4 | 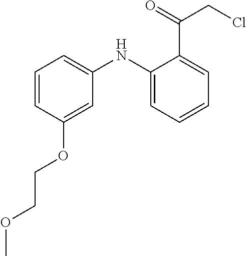 [SMILES: COCCOc1cccc(Nc2ccccc2C(=O)CCl)c1] | 2-chloro-1-(2-((3-(2- methoxyethoxy)phenyl)amino)phenyl) ethanone |
| 5 | 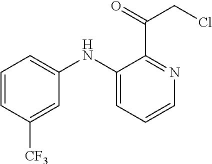 [SMILES: O=C(CCl)c1ncccc1Nc1cccc(C(F)(F)F)c1] | 2-chloro-1-(3-((3- (trifluoromethyl)phenyl)amino)pyridin-2- yl)ethanone |
| 6 | 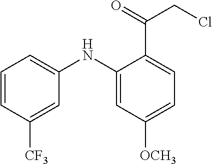 [SMILES: COc1ccc(C(=O)CCl)c(Nc2cccc(C(F)(F)F)c2)c1] | 2-chloro-1-(4-methoxy-2-((3- (trifluoromethyl)phenyl)amino)phenyl) ethanone |
| 7 | 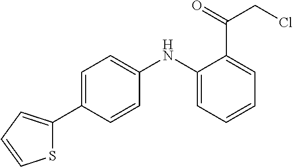 [SMILES: O=C(CCl)c1ccccc1Nc1ccc(-c2cccs2)cc1] | 2-chloro-1-(2-((4-(thiophen-2- yl)phenyl)amino)phenyl)ethanone |
| 8 | 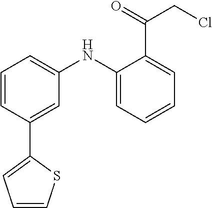 [SMILES: O=C(CCl)c1ccccc1Nc1cccc(-c2cccs2)c1] | 2-chloro-1-(2-((3-(thiophen-2- yl)phenyl)amino)phenyl)ethanone |
| 9 | 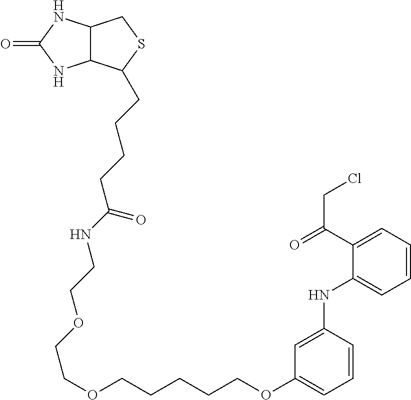 [SMILES: O=C(CCCCC1SCC2NC(=O)NC21)NCCOCCOCCCCCOc1cccc(Nc2ccccc2C(=O)CCl)c1] | N-(2-(2-((5-(3-((2-(2- chloroacetyl)phenyl)amino)phenoxy)pentyl) oxy)ethoxy)ethyl)-5-(2-oxohexahydro-1H- thieno[3,4-d]imidazol-4-yl)pentanamide |
| 10 | 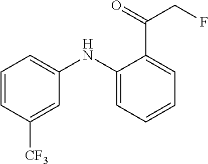 [SMILES: O=C(CF)c1ccccc1Nc1cccc(C(F)(F)F)c1] | 2-fluoro-1-(2-((3- (trifluoromethyl)phenyl)amino)phenyl) ethanone |
| 11 | 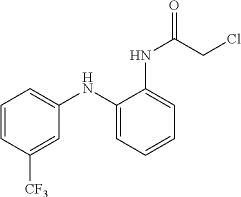 [SMILES: O=C(CCl)Nc1ccccc1Nc1cccc(C(F)(F)F)c1] | 2-chloro-N-(2-((3- (trifluoromethyl)phenyl)amino)phenyl) acetamide |
| 12 | 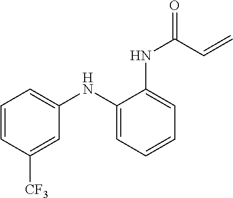 [SMILES: C=CC(=O)Nc1ccccc1Nc1cccc(C(F)(F)F)c1] | N-(2-((3- (trifluoromethyl)phenyl)amino) phenylacrylamide |

Those skilled in the art will recognize that the species listed or illustrated herein are not exhaustive, and that additional species within the scope of these defined terms may be selected.

### Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the invention are sterile compositions. Pharmaceutical compositions may be prepared using compounding techniques known or that become available to those skilled in the art.

Sterile compositions are also contemplated by the invention, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the invention may be administered by a suitable route of delivery, such as oral, parenteral, rectal, nasal, topical, or ocular routes, or by inhalation. Preferably, the compositions are formulated for intravenous or oral administration.

For oral administration, the compounds the invention may be provided in a solid form, such as a tablet or capsule, or as a solution, emulsion, or suspension. To prepare the oral compositions, the compounds of the invention may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present invention are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the invention may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

The compounds of the present disclosure can be described as embodiments in any of the following enumerated clauses. It will be understood that any of the embodiments described herein can be used in connection with any other embodiments described herein to the extent that the embodiments do not contradict one another.

Clause 1. A compound of Formula (1a)

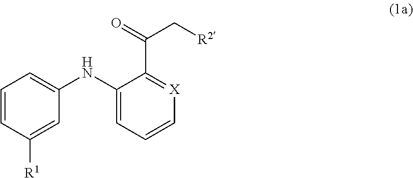

wherein

R¹ is C(R)₃ or lower (C₁-C₆) alkyl, where one or more of the carbon atoms in lower (C₁-C₆) alkyl are replaced by oxygen; and where R is a halogen;

R^{2′} halogen or lower (C₁-C₆) alkyl, where one or more of the hydrogens in lower (C₁-C₆) alkyl are replaced with a halogen; and

X is carbon or a heteroatom independently selected from nitrogen, oxygen and sulfur;

or a pharmaceutically acceptable salt thereof.

Clause 2. A compound of Formula (1)

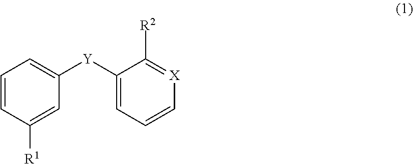

wherein

Y is selected from the group consisting of —C(O)—, —NH—, —O—, —S—, —CH₂—, and —S(O)₂—;

R¹ is C(R)₃ or lower (C₁-C₆) alkyl, where one or more of the carbon atoms in lower (C₁-C₆) alkyl are replaced by oxygen; and where R is a halogen;

R² is selected from the group consisting of —CH₂CN, —CN, —NHC(O)CHCH₂, —S(O)₂CHCH₂, —NHC(O)CHCHCH₂N(CH₃)₂, —NHC(O)CCH, —C(O)CH₂R^{2′}, and —NHC(O)CH₂R^{2′}, where R^{2′} is halogen or lower (C₁-C₆) alkyl, where one or more the hydrogens in lower (C₁-C₆) alkyl are replaced with a halogen; and

X is carbon or a heteroatom independently selected from nitrogen, oxygen and sulfur;

or a pharmaceutically acceptable salt thereof.

Clause 3. A compound of Formula (1b)

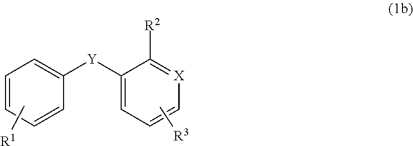

wherein

Y is selected from the group consisting of —C(O)—, —NH—, —O—, —S—, —CH₂—, and —S(O)₂;

R¹ is selected from the group consisting of —C(R)₃, thiophene, —O(CH₂)ₙ(OCH₂CH₂)ₘNH-biotin, and lower (C₁-C₆) alkyl, where one or more of the carbon atoms in lower (C₁-C₆) are replaced by oxygen, n is an integer from 1 to 6 and m is an integer from 1 to 3; and where R is a halogen;

R² is selected from the group consisting of —CH₂CN, —CN, —NHC(O)CHCH₂, —S(O)₂CHCH₂, —NHC(O)CHCHCH₂N(CH₃)₂, —NHC(O)CCH, —C(O)CH₂R^{2′}, and —NHC(O)CH₂R^{2′}, where R^{2′} is halogen or lower (C₁-C₆) alkyl, where one or more the hydrogens in lower (C₁-C₆) alkyl are replaced with a halogen;

R³ is H or C₁-C₆ alkoxy,

X is carbon or a heteroatom independently selected from nitrogen, oxygen and sulfur;

or a pharmaceutically acceptable salt thereof.

Clause 4. The compound or pharmaceutically acceptable salt thereof of any of the preceding clauses, wherein lower (C₁-C₆) alkyl is methyl, ethyl, or n-propyl.

Clause 5. The compound or pharmaceutically acceptable salt thereof of any of the preceding clauses, wherein R^{2′} is a halogen.

Clause 6. The compound or pharmaceutically acceptable salt thereof of any of the preceding clauses, wherein R^{2′} is chloro.

Clause 7. The compound or pharmaceutically acceptable salt thereof of any of the preceding clauses, wherein R¹ is C(R)₃ where R is a halogen.

Clause 8. The compound or pharmaceutically acceptable salt thereof of any o of the preceding clauses, wherein X is carbon or nitrogen.

Clause 9. The compound or pharmaceutically acceptable salt thereof of any of the preceding clauses, wherein X is carbon.

Clause 10. The compound or pharmaceutically acceptable salt thereof of any of clauses 1-8, wherein X is nitrogen.

Clause 11. The compound or pharmaceutically acceptable salt thereof of any of the preceding clauses, wherein Y is —NH—.

Clause 12. The compound or pharmaceutically acceptable salt thereof of any of the preceding clauses, wherein R² is —C(O)CH₂R^{2′}.

Clause 13. The compound or pharmaceutically acceptable salt thereof of the preceding clauses, wherein R^{2′} is a halogen.

Clause 14. The compound or pharmaceutically acceptable salt thereof of the preceding clauses, wherein R^{2′} is chloro.

Clause 15. The compound or pharmaceutically acceptable salt thereof of the preceding clauses, wherein R³ is a H.

Clause 16. The compound or pharmaceutically acceptable salt thereof of the any of clauses 1-14, wherein R³ is a alkoxy.

Clause 17. The compound or pharmaceutically acceptable salt thereof of the any of clauses 1-14, wherein R³ is methoxy.

Clause The compound or pharmaceutically acceptable salt thereof of the preceding clauses, wherein R¹ is thiophene.

Clause 19. The compound or pharmaceutically acceptable salt thereof of any of clauses 1-17, wherein R¹ is —O(CH₂)ₙ(OCH₂CH₂)ₘNH-biotin.

Clause 20. The compound or pharmaceutically acceptable salt thereof of the preceding clauses, wherein m is 2.

Clause 21. The compound or pharmaceutically acceptable salt thereof of the preceding clauses, wherein n is 5.

Clause 22. A compound selected from the group consisting of 2-chloro-1-(2-((3-(trifluoromethyl)phenyl) amino)phenyl)ethanone, 2-chloro-1-(2-((3-(2-methoxyethoxy)phenyl)amino)phenyl)ethanone, 2-chloro-1-(3-((3-(trifluoromethyl)phenyl)amino)pyridin-2-yl)ethanone, and 2-chloro-1-(4-methoxy-2-((3-(trifluoromethyl)phenyl)amino)phenyl)ethanone, or a pharmaceutically acceptable salt thereof.

Clause 23. A compound selected from the group consisting of 2-chloro-1-(2-((3-(trifluoromethyl)phenyl) amino)phenyl)ethanone, 2-chloro-1-(2-((3-(2-methoxyethoxy)phenyl)amino)phenyl)ethanone, 2-chloro-1-(3-((3-(trifluoromethyl)phenyl)amino)pyridin-2-yl)ethanone, 2-chloro-1-(4-methoxy-2-((3-(trifluoromethyl)phenyl)amino)phenyl)ethanone, 2-chloro-1-(2-((4-(thiophen-2-yl)phenyl)amino)phenyl)ethanone, 2-chloro-1-(2-((3-(thiophen-2-yl)phenyl)amino)phenyl)ethanone, N-(2-(2-((5-(3-((2-(2-chloroacetyl)phenyl)amino)phenoxy)pentyl)oxy)ethoxy)ethyl)-5-(2-oxohexahydro-1H-thieno[3,4-d]imidazol-4-yl)pentanamide,

2-fluoro-1-(2-((3-(trifluoromethyl)phenyl)amino)phenyl) ethanone, 2-chloro-N-(2-((3-(trifluoromethyl)phenyl)amino)phenyl) acetamide, and N-(2-((3-(trifluoromethyl)phenyl)amino)phenylacrylamide; or a pharmaceutically acceptable salt thereof.

Clause 24. A pharmaceutical composition comprising a compound according to of the preceding clauses, or a pharmaceutically acceptable salt thereof, and at least one of a pharmaceutically acceptable carrier, diluent or excipient.

Clause 25. A method to treat cancer in a mammal in need thereof, comprising administering an effective amount of a compound of Formula (1a)

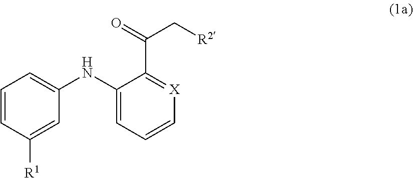

wherein

R¹ is C(R)₃ or lower (C₁-C₆) alkyl, where one or more of the carbon atoms in lower (C₁-C₆) alkyl are replaced by oxygen; and where R is a halogen;

R^{2′} is halogen or lower (C₁-C₆) alkyl, where one or more of the hydrogens in lower (C₁-C₆) alkyl are replaced with a halogen; and

X is carbon or a heteroatom independently selected from nitrogen, oxygen and sulfur;

or a pharmaceutically acceptable salt thereof.

Clause 26. A method to treat cancer in a mammal in need thereof, comprising administering an effective amount of a compound of Formula (1)

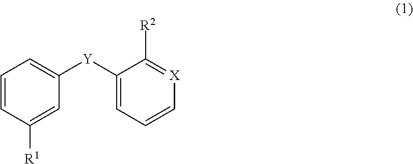

wherein

Y is selected from the group consisting of —C(O)—, —NH—, —O—, —S—, —CH₂—, and S(O)₂—;

R¹ is C(R)₃ or lower (C₁-C₆) alkyl, where one or more of the carbon atoms in lower (C₁-C₆) alkyl are replaced by oxygen; and where R is a halogen;

R² is selected from the group consisting of —CH₂CN, —CN, —NHC(O)CHCH₂, —S(O)₂CHCH₂, —NHC(O)CHCHCH₂N(CH₃)₂, —NHC(O)CCH, —C(O)CH₂R^{2′} and —NHC(O)CH₂R²⁹, where R^{2′} is halogen or lower (C₁-C₆) alkyl, where one or more the hydrogens in lower (C₁-C₆) alkyl are replaced with a halogen; and

X is carbon or a heteroatom independently selected from nitrogen, oxygen and sulfur;

or a pharmaceutically acceptable salt thereof.

Clause 27. A method to treat cancer in a mammal in need thereof, comprising administering an effective amount of a compound Formula (1b)

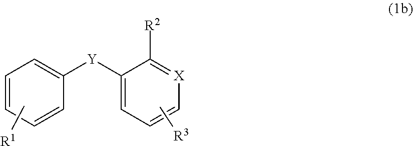

wherein

Y is selected from the group consisting of —C(O)—, —NH—, —O—, —S—, —CH₂—, and —S(O)₂;

R¹ is selected from the group consisting of —C(R)₃, thiophene, —O(CH₂)ₙ(OCH₂CH₂)ₘNH-biotin, and lower (C₁-C₆) alkyl, where one or more of the carbon atoms in lower (C₁-C₆) are replaced by oxygen, n is an integer from 1 to 6 and m is an integer from 1 to 3; andwhere R is a halogen;

R² is selected from the group consisting of —CH₂CN, —CN, —NHC(O)CHCH₂, —S(O)₂CHCH₂, —NHC(O)CHCHCH₂N(CH₃)₂, —NHC(O)CCH, —C(O)CH₂R^{2′}, and —NHC(O)CH₂R^{2′}, where R^{2′} is halogen or lower (C₁-C₆) alkyl, where one or more the hydrogens in lower (C₁-C₆) alkyl are replaced with a halogen;

R³ is H or C₁-C₆ alkoxy,

X is carbon or a heteroatom independently selected from nitrogen, oxygen and sulfur;

or a pharmaceutically acceptable salt thereof.

Clause 28. A method to modulate a protein-protein interaction between a Yes-associated protein (YAP) transcriptional coactivator and a TEA/ATS (TEAD) transcription factor in a cell, comprising contacting the cell with an effective amount of the compound of Formula (1a)

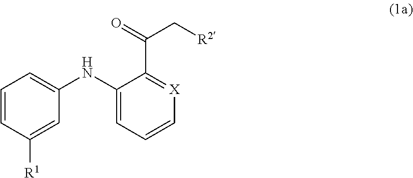

wherein

R¹ is C(R)₃ or lower (C₁-C₆) alkyl, where one or more of the carbon atoms in lower (C₁-C₆) alkyl are replaced by oxygen; and where R is a halogen;

R^{2′} is halogen or lower (C₁-C₆) alkyl, where one or more of the hydrogens in lower (C₁-C₆) alkyl are replaced with a halogen; and

X is carbon or a heteroatom independently selected from nitrogen, oxygen and sulfur;

or a pharmaceutically acceptable salt thereof.

Clause 29. A method to modulate a protein-protein interaction between a Yes-associated protein (YAP) transcriptional coactivator and a TEA/ATS (TEAD) transcription factor in a cell, comprising contacting the cell with an effective amount of the compound of Formula (1)

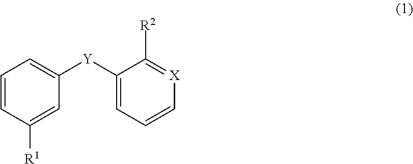

wherein

Y is selected from the group consisting of —C(O)—, —NH—, —O—, —S—, —CH₂—, and S(O)₂—;

R¹ is C(R)₃ or lower (C₁-C₆) alkyl, where one or more of the carbon atoms in lower (C₁-C₆) alkyl are replaced by oxygen; and where R is a halogen;

R² is selected from the group consisting of —CH₂CN, —CN, —NHC(O)CHCH₂, —S(O)₂CHCH₂, —NHC(O)CHCHCH₂N(CH₃)₂, —NHC(O)CCH, —C(O)CH₂R^{2′}, and —NHC(O)CH₂R^{2′}, where R^{2′} is halogen or lower (C₁-C₆) alkyl, where one or more the hydrogens in lower (C₁-C₆) alkyl are replaced with a halogen; and

X is carbon or a heteroatom independently selected from nitrogen, oxygen and sulfur;

or a pharmaceutically acceptable salt thereof.

Clause 30. A method to modulate a protein-protein interaction between a Yes-associated protein (YAP) transcriptional coactivator and a TEA/ATS (TEAD) transcription factor in a cell, comprising contacting the cell with an effective amount of the compound Formula (1b)

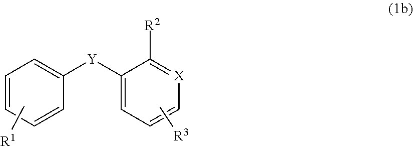

wherein

Y is selected from the group consisting of —C(O)—, —NH—, —O—, —S—, —CH₂—, and —S(O)₂;

R¹ is selected from the group consisting of —C(R)₃, thiophene, —O(CH₂)ₙ(OCH₂CH₂)ₘNH-biotin, and lower (C₁-C₆) alkyl, where one or more of the carbon atoms in lower (C₁-C₆) are replaced by oxygen, n is an integer from 1 to 6 and m is an integer from 1 to 3; and where R is a halogen;

R² is selected from the group consisting of —CH₂CN, —CN, —NHC(O)CHCH₂, —S(O)₂CHCH₂, —NHC(O)CHCHCH₂N(CH₃)₂, —NHC(O)CCH, —C(O)CH₂R^{2′}, and —NHC(O)CH₂R^{2′}, where R^{2′} is halogen or lower (C₁-C₆) alkyl, where one or more the hydrogens in lower (C₁-C₆) alkyl are replaced with a halogen;

R³ is H or C₁-C₆ alkoxy,

X is carbon or a heteroatom independently selected from nitrogen, oxygen and sulfur;

or a pharmaceutically acceptable salt thereof.

Clause 31. The method of any of clauses 25-30, wherein lower (C₁-C₆) alkyl is methyl, ethyl, or n-propyl.

Clause 32. The method of any of clauses 25-31, wherein R^{2′} is a halogen.

Clause 33. The method of any of clauses 25-32, wherein R^{2′} is chloro.

Clause 34. The method of any of clauses 25-33, wherein R′ is C(R)₃ where R is a halogen.

Clause 35. The method of any of clauses 25-34, wherein X is carbon or nitrogen.

Clause 36. The method of any of clauses 25-35, wherein X is carbon.

Clause 37. The method of any of clauses 25-35, wherein X is nitrogen.

Clause 38. The method of any of clauses 25-37, wherein Y is —NH—.

Clause 39. The method of any of clauses 25-38, wherein R² is —C(O)CH₂R^{2′}.

Clause 40. The method of any of clauses 25-39, wherein R^{2′} is a halogen.

Clause 41. The method any of clauses 25-39, wherein R^{2′} is chloro.

Clause 42. The method of any of clauses 25-41, wherein R³ is a H.

Clause 43. The method of any of clauses 25-41, wherein R³ is a alkoxy.

Clause 44. The method of any of clauses 25-41, wherein R³ is methoxy.

Clause 45. The method of any of clauses 25-44, wherein R¹ is thiophene.

Clause 46. The method of any of clauses 25-44, wherein R′ is —O(CH₂)ₙ(OCH₂CH₂)ₘNH-biotin.

Clause 47. The method of any of clauses 25-46, wherein m is 2.

Clause 48. The method of any of clauses 25-47, wherein n is 5.

Clause 49. The method of any of clauses 25-48, wherein the cancer is a solid tumor, lung cancer, thyroid cancer, skin cancer, ovarian cancer, colon cancer, rectal cancer, prostate cancer, pancreatic cancer, esphogal cancer, liver cancer, or breast cancer.

Clause 50. The method of any of clauses 25-49, wherein the cancer is glioblastoma.

Clause 51. A method to identify a compound capable of forming an adduct with a conserved cysteine in a protein, the method, comprising identifying a protein having a conserved cysteine in an allosteric pocket and performing solvent molecular dynamics simulations on a compound.

Clause 52. Use of a compound according to any one of clauses 1-23, or a pharmaceutically acceptable salt thereof, in the manufacture of a medicament for use in treating cancer in a patient.

Clause 53. The use of clause 52, wherein the cancer is a solid tumor, lung cancer, thyroid cancer, skin cancer, ovarian cancer, colon cancer, rectal cancer, prostate cancer, pancreatic cancer, esphogal cancer, liver cancer, or breast cancer.

Clause 54. The use of clause 52, wherein the cancer is glioblastoma.

Clause 55. A compound according to any one of clauses 1-23, or a pharmaceutically acceptable salt thereof, for treating cancer in a patient.

Clause 56. The compound of clause 55, wherein the cancer is a solid tumor, lung cancer, thyroid cancer, skin cancer, ovarian cancer, colon cancer, rectal cancer, prostate cancer, pancreatic cancer, esphogal cancer, liver cancer, or breast cancer.

Clause 57. The compound of clause 55, wherein the cancer is glioblastoma.

### Chemical Synthesis

Exemplary chemical entities useful in methods of the description will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups.

Abbreviations: The examples described herein use materials, including but not limited to, those described by the following abbreviations known to those skilled in the art:

| | |
|---|---|
| g | grams |
| eq | equivalents |
| mmol or mM | millimoles |
| mL | milliliters |
| EtOAc or EA | ethyl acetate |
| MHz | megahertz |
| Ppm | parts per million |
| s | singlet |
| d | doublet |
| t | triplet |
| Br | broad |
| m | multiplet |
| dd | doublet of doublets |
| Hz | hertz |
| THF | tetrahydrofuran |
| ° C. | degrees Celsius |
| J | coupling constant |
| CDCl₃ | deuterated chloroform |
| CD₃OD | deuterated methanol |
| min | minutes |
| h | hours |
| ns | nanosecond |
| ps | picosecond |
| TLC | thin layer chromatography |
| HPLC | high performance liquid chromatography |
| M | molar |
| ESIMS | electrospray ionization mass spectrum |
| LRMS | low resolution mass spectrometry |
| m/z | mass-to-charge ratio |
| Ms | methanesulfonyl |
| μM | micromolar |
| DMF | N,N-dimelhylformamide |
| NCS | N-chlorosuccinimide |
| DIEA | diisopropylethylamine |
| TEA | triethylamine |
| DCM | dichloromethane |
| HATU | 1-[Bis(dimethylamino)methylene]-1H-1,2,3- |
| | triazolo[4,5-b]pyridinium 3-oxid |
| | hexafluorophosphate |
| Ph | phenyl |
| boc | tert-Butyloxycarbonyl |
| TBS | tert-butyldimethylsilyl |
| TBAF | tetrabutylammoniumfluoride |
| DIAD | Diisopropyl azodicarboxylate |
| Hex | hexanes |
| Ac | acetyl |
| PE | petroleum ether |
| EtOH | ethanol |
| MeOH | methanol |
| MeCN | acetonitrile |
| xPhos | 2-Dicyclohexylphosphino-2′,4′,6′- |
| | triisopropylbiphenyl |
| TMS | trimethylsilyl |
| Tf | triflate |
| Bn | benzyl |
| atm | atmosphere |
| dba | dibenzylideneacetone |

Synthesis

All chemicals were purchased from either Aldrich or Acros and used as received. Column chromatography was carried out with silica gel (25-63μ). Mass spectra were measured on an Agilent 6520 Accurate Mass Q-TOF instrument. ¹H NMR spectra were recorded in CDCl₃ or Methanol-d4 on a Bruker 500 MHz spectrometer. Chemical shifts are reported using residual CHCl₃ or MeOH as internal references. All compounds that were evaluated in biological assays had >95% purity by HPLC.

### Example 1

### 2-((3-(trifluoromethyl)phenyl)amino)benzoic Acid

Compound 1 was purchased from a commercial source. ¹H NMR (400 MHz, CDCl₃): δ 9.42 (s, 1H), 8.09-8.07 (dd, J=8.0 Hz, J=1.2 Hz, 1H), 7.51 (s, 1H), 7.49-7.40 (m, 3H), 7.36-7.34 (m, 1H), 7.27-7.25 (m, 2H), 6.87-6.83 (t, J=8.0 Hz, 1H).

### Example 2

### 2-chloro-1-(2-((3-(trifluoromethyl)phenyl)amino)phenyl)ethanone

A solution of 2-((3-(trifluoromethyl)phenyl)amino)benzoic acid (1, 200 mg, 0.71 mmol) in SOCl₂ (6 mL) was refluxed for 1 h. The mixture was evaporated to dryness. The residue was dissolved in MeCN (10 mL) and cooled to 0° C., followed by the addition of TMSCHN₂ (1.07 mmol, 0.5 mL) It was stirred for 1 h, and conc. HCl (0.5 mL) was added to the mixture at 0° C. The mixture was stirred for 0.5 h. The mixture was quenched with NaHCO₃(aq) and diluted with water. The mixture was extracted with EA (15 mL×2). The combined organic layers were dried with Na₂SO₄, filtered and concentrated. The residue was purified by Prep-TLC (PE/EA=5/1) to give 2-chloro-1-(2-((3-(trifluoromethyl)phenyl)amino)phenyl)ethanone (6.4 mg, 2.8%); ¹H NMR (400 MHz, CDCl₃): δ10.43 (s, 1H), 7.79-7.64 (dd, J=4.0 Hz, J=0.8 Hz, 1H), 7.51-7.47 (m, 1H), 7.45-7.41 (m, 1H), 7.38-7.36 (m, 3H), 7.29-7.26 (m, 1H), 6.85-6.82 (m, 1H), 4.75 (s, 1H); ¹³C NMR (100 MHz, CDCl₃) δ 193.41, 147.80, 140.67, 135.66, 132.16, 131.86, 130.04, 125.86, 125.20, 122.49, 120.71, 120.67, 120.63, 119.33, 119.30, 119.26, 117.84, 116.87, 114.65, 46.53. LRMS calculated for C15H12ClF3NO⁺ [M+H]⁺, 314.05 found 314.0.

### Example 3

### 1-(2-((3-(trifluoromethyl)phenyl)amino)phenyl)propan-1-one

To a solution of N-methoxy-N-methyl-2-((3-(trifluoromethyephenyl)amino)benzamide (100 mg, 0.31 mmol) in THF (5 mL) was added 1 M EtMgBr in THF (1.8 mL) at −78° C. under N₂. The resulting mixture was warmed to rt and stirred for 5 h. The reaction mixture was quenched with sat. NH₄Cl (20 mL), and extracted with EA (5 mL×3). The organic phase was washed with brine, dried over Na₂SO₄, and filtered. The filtrate was concentrated. The residue was purified by prep-TLC (PE/EA=20/1) to give 1-(2-((3-(trifluoromethyl)phenyl)amino)phenyl)propan-1-one (15 mg, 16.5%); ¹H NMR (400 MHz, CDCl₃): δ10.68 (s, 1H), 7.89 (d, J=8.0 Hz, 1H), 7.51 (s, 1H), 7.30-7.46 (m, 5H), 6.83 (t, J=7.2 Hz, 1H), 3.07 (q, 2H), 1.24 (t, J=7.2 Hz, 3H); ¹³C NMR (100 MHz, CDCl₃) δ 204.20, 146.57, 141.44, 134.38, 131.59, 129.87, 125.03, 119.78, 119.72, 118.52, 118.49, 117.79, 114.56, 32.70, 8.69. LRMS calculated for C16H15F3NO⁺ [M+H]⁺, 293.3 found 294.1.

### Example 4

### 1-(2-((3-(2-methoxyethoxy)phenyl)amino)phenyl)ethanone

A mixture of 3-(2-methoxyethoxy)aniline (500 mg, 2.99 mmol), 1-(2-bromophenyl) ethanone (625 mg, 3.14 mmol), Pd₂(dba)₃ (275 mg, 0.30 mmol), xphos (286 mg, 0.60 mmol), and Cs₂CO₃ (1.47 g, 4.50 mmol) in dioxane (10 mL) was heated to 90° C. under N₂, and stirred for 2 h. The reaction mixture was cooled to room temperature, and filtered over Celite. The filtrate was concentrated. The residue was dissolved in EA (40 mL), washed with brine, and dried over Na₂SO₄. The solution was filtered, and the filtrate was concentrated. The residue was purified by column chromatography (PE-PE/EA=5/1) to give 1-(2-((3-(2-methoxyethoxy)phenyl)amino)phenyl)ethanone (710 mg, 83.2%); ¹H NMR (400 MHz, CDCl₃): δ 10.51 (s, 1H), 7.81 (d, J=8.0 Hz, 1H), 7.30-7.31 (m, 2H), 7.23 (t, J=8.0 Hz, 1H), 6.84-6.85 (m, 2H), 6.68-6.76 (m, 2H), 4.11 (t, J=4.8 Hz, 2H), 3.75 (t, J=4.8 Hz, 2H), 3.45 (s, 3H), 2.64 (s, 3H). LRMS calculated for C17H20NO3+ [M+H]⁺, 286.3 found 286.2.

### 2-chloro-1-(2-((3-(2-methoxyethoxy)phenyl)amino)phenyl)ethanone

To a mixture of 1-(2-((3-(2-methoxyethoxy)phenyl)amino)phenyl)ethanone (50 mg, 0.18 mmol) in DCM (3 mL) were added DIEA (67 mg, 0.52 mmol) and TMSOTf (58 mg, 0.26 mmol) at 0° C. under N₂. The resulting mixture was warmed to room temperature and stirred for 5 h. NCS (24 mg, 0.18 mmol) was added and the mixture was stirred for another 2 h. It was quenched with water (10 mL), and the mixture was extracted with DCM (5 mL×3). The organic phase was washed with brine, dried over Na₂SO₄, and filtered. The filtrate was concentrated. The residue was purified by prep. TLC (PE/EA=5/1) to give 2-chloro-1-(2-((3-(2-methoxyethoxy)phenyl)amino) phenyl)ethanone (5.1 mg, 8.9%); ¹H NMR (400 MHz, CDCl₃): δ 10.35 (s, 1H), 7.77 (d, J=8.0 Hz, 1H), 7.22-7.39 (m, 4H), 7.07 (d, J=2.8 Hz, 1H), 6.84 (t, J=7.2 Hz, 1H), 6.63-6.65 (m, 1H), 4.76 (s, 2H), 4.08 (t, J=4.8 Hz, 2H), 3.74 (t, J=4.8 Hz, 2H), 3.45 (s, 3H); ¹³C NMR (100 MHz, CDCl₃): δ 193.13, 157.99, 147.15, 137.90, 135.36, 131.25, 130.56, 117.91, 117.49, 115.51, 110.85, 109.43, 70.95, 67.75, 59.25, 46.55. LRMS calculated for C17H19ClNO3+ [M+H]⁺, 320.8 found 320.0.

### Example 5

### 1-(3-((3-(trifluoromethyl)phenyl)amino)pyridin-2-yl)ethanone

A mixture of 1-(3-bromopyridin-2-yl)ethanone (200 mg, 1.00 mmol), 3-(trifluoromethyl) aniline (161 mg, 1.00 mmol), Pd₂(dba)₃ (92 mg, 0.10 mmol), xphos (95 mg, 0.20 mmol), and Cs₂CO₃ (489 mg, 1.50 mmol) in dioxane (10 mL) was heated to 90° C. under N₂. The mixture was stirred for 2 h. The reaction mixture was cooled to room temperature, and filtered through Celite. The filtrate was concentrated. The residue was dissolved in EA (40 mL), and the mixture was washed with brine, dried over Na₂SO₄, and filtered. The filtrate was concentrated. The residue was purified by column chromatography (PE-PE/EA=20/1) to give 1-(3-((3-trifluoromethyl) phenyl) amino)pyridin-2-yl)ethanone (160 mg, 57.1%); ¹H NMR (400 MHz, CDCl₃): δ 10.43 (s, 1H), 8.13 (d, J=4.0 Hz, 1H), 7.59 (d, J=8.0 Hz, 1H), 7.47-7.51 (m, 2H), 7.39 (t, J=6.4 Hz, 2H), 7.26-7.30 (m, 1H), 2.79 (s, 3H). LRMS calculated for C14H12F3N2O⁺ [M+H]⁺, 281.3 found 281.1.

### 2-chloro-1-(3-((3-(trifluoromethyl)phenyl)amino)pyridin-2-yl)ethanone)

The procedure was the same as compound 4 to give compound 5. ¹H NMR (400 MHz, CDCl₃): δ 10.21 (s, 1H), 8.11-8.10 (dd, J=4.0 Hz, J=1.2 Hz, 1H), 7.61-7.58 (m, 1H), 7.53-7.49 (m, 2H), 7.44-7.39 (m, 2H), 7.34-7.31 (m, 1H), 5.20 (s, 2H). ¹³C NMR (100 MHz, CDCl₃): δ 195.58, 143.74, 139.61, 139.07, 133.56, 132.42, 132.10, 130.32, 129.07, 126.07, 125.05, 122.34, 121.64, 121.47, 121.39, 119.51, 119.47, 48.03. LRMS calculated for C14H11ClF3N2O⁺[M+H]⁺, 315.04 found 315.0.

### Example 6

### Methyl 4-methoxy-2-((3-(trifluoromethyl)phenyl)amino)benzoate

A mixture of methyl-2-amino-4-methoxybenzoate (1.0 g, 5.5 mmol), 1-iodo-3-(trifluoromethyl)benzene (1.8 g, 6.6 mmol), Pd₂(dba)₃ (504 mg, 0.55 mmol), xphos (286 mg, 0.60 mmol), and Cs₂CO₃ (3.6 g, 110 mmol) in dioxane (40 mL) was stirred at 90° C. under N₂ overnight. The reaction mixture was cooled to room temperature, and filtered over Celite. The filtrate was concentrated. The residue was dissolved in ethyl acetate (40 mL), washed with brine, and dried over Na₂SO₄. The solution was filtered, and the filtrate was concentrated. The residue was purified by column chromatography (PE-PE/EA=5/1) to give methyl 4-methoxy-2-((3-(trifluoromethyl) phenyl) amino)benzoate (1.12 g, 62.7%); ¹H NMR (400 MHz, CDCl₃): δ 9.75 (s, 1H), 7.95-7.92 (d, J=8.8 Hz, 1H), 7.52 (s, 1H), 7.44-7.43 (m, 2H), 7.32-7.30 (d, J=6.8 Hz, 1H), 6.73 (d, J=6.4 Hz, 1H), 6.39-6.36 (dd, J=9.2 Hz, J=2.4 Hz, 1H), 3.88 (s, 3H), 3.76 (s, 3H). LRMS calculated for C17H20NO₃⁺[M+H]⁺, 326.1 found 326.1.

### 4-methoxy-2-((3-(trifluoromethyl)phenyl)amino)benzoic Acid

To a mixture of methyl 4-methoxy-2-((3-(trifluoromethyl)phenyl)amino)benzoate (TED-589-1, 1.12 g, 3.38 mmol) in a mixture of dioxane/water (10 mL/10 mL) was added LiOH.H₂O (1.43 g, 33.8 mmol) at rt and the mixture was stirred for 2 h. The mixture was acidified to pH=6 with 1 M HCl, and the organic phase was extracted with ethyl acetate (10 mL×2). The organic phase was washed with brine, dried over Na₂SO₄, and filtered. The filtrate was concentrated. The residue was purified by column chromatography (DCM/MeOH=10/1) to give 4-methoxy-2-((3-(trifluoromethyl) phenyl)amino)benzoic acid (1.07 g, 99% yield); LRMS calculated for C17H20NO3+ [M+H]⁺, 312.1 found 312.1.

### N,4-dimethoxy-N-methyl-2-((3-(trifluoromethyl)phenyl)amino)benzamide

To a solution of 4-methoxy-2-((3-(trifluoromethyl)phenyl)amino)benzoic acid (TED-589-2, 1.07 g, 3.46 mmol), N,O-dimethylhydroxylamine hydrochloride (503 mg, 5.19 mmol) and HATU (1.97 g, 5.19 mmol) in DMF (20 mL) was added N-ethyl-N-isopropylpropan-2-amine (880 mg, 7.0 mmol). The mixture was stirred at room temperature for 2 h. After completion of the reaction, ethyl acetate was added to the mixture. Then the mixture was washed with water (100 mL×2) and brine (100 mL) The organic phase was dried over Na₂SO₄, filtered and concentrated. The residue was purified by column chromatography (PE/EA=3:1) to give N,4-dimethoxy-N-methyl-2-((3-(trifluoromethyl) phenyl) amino)benzamide as a yellow oil (1.12 g, 91.3% yield); ¹H NMR (400 MHz, CDCl₃): δ 8.44 (s, 1H), 7.54-7.52 (d, J=8.8 Hz, 1H), 7.41-7.40 (d, J=9.2 Hz, 1H), 7.38-7.36 (d, J=8.0 Hz, 1H), 7.29-7.26 (m, 1H), 7.20-7.18 (d, J=7.6 Hz, 1H), 6.88 (d, J=2.8 Hz, 1H), 6.46-6.43 (dd, J=11.2 Hz, J=2.4 Hz, 1H), 3.77 (s, 3H), 3.60 (s, 3H), 3.36 (s, 3H). LRMS calculated for C17H20NO3+ [M+H]⁺, 355.1 found 355.1.

### 1-(4-methoxy-2-((3-(trifluoromethyl)phenyl)amino)phenyl)ethanone

To a solution of N,4-dimethoxy-N-methyl-2-(43-(trifluoromethyephenyl)amino)benzamide (400 mg, 1.13 mmol) in dry THF (20 mL) under N₂ was added methylmagnesium bromide (10 ml 1.0M in THF, 10.0 mmol) at 0° C. The mixture was stirred at 0° C. for 0.5 h and then at room temperature for 2 h. The reaction mixture was quenched by the addition of saturated aqueous NH₄Cl. The organic phase was extracted with ethyl acetate. The combined ethyl acetate layers were washed with brine, dried over anhydrous sodium sulfate, and concentrated. The residue was purified by column chromatography (PE/EA=6:1) to give the desired product as a yellow oil (310 mg, 88.5% yield); ¹H NMR (400 MHz, CDCl₃): δ 10.90 (s, 1H), 7.80-7.77 (d, J=8.8 Hz, 1H), 7.55 (s, 1H), 7.46-7.34 (m, 1H), 7.35-7.33 (d, J=6.8 Hz, 1H), 6.70 (d, J=2.4 Hz, 1H), 6.38-6.35 (dd, J=8.8 Hz, J=2.4 Hz, 1H), 3.76 (s, 3H), 2.59 (s, 3H). LRMS calculated for C16H13F3NO⁺ [M+H]⁺, 310.1 found 310.1.

### 2-chloro-1-(4-methoxy-2-((3-(trifluoromethyl)phenyl)amino)phenyl)ethanone

To a mixture of 1-(4-methoxy-2-((3-(trifluoromethyl)phenyl)amino)phenyl)ethanone (TED-589-4, 310 mg, 10 mmol) in dichoromethane (DCM) (10 mL) were added DIEA (256 mg, 2.0 mmol) and TMSOTf (266 mg, 1.2 mmol) at 0° C. under N₂. The resulting mixture the mixture was stirred for another 2 h. It was quenched with water (10 mL), and the mixture was extracted with DCM (5 mL×3). The organic phase was washed with brine, dried over Na₂SO₄, and filtered. The filtrate was concentrated. The residue was purified by prep. TLC (PE/EA=5/1) to give the crude product (207 mg). The compound was further purified by reverse HPLC Gilson to afford the desired product as a yellow solid (78 mg, 22.6% yield); ¹H NMR (400 MHz, CDCl₃): δ 10.72 (s, 1H), 7.73-7.70 (d, J=9.2 Hz, 1H), 7.56 (s, 1H) 7.50-7.38 (m, 3H), 6.69 (s, 1H), 6.40-6.38 (d, J=8.8 Hz, 1H), 4.67 (s, 2H), 3.77 (s, 3H); ¹³C NMR (100 MHz, CDCl₃): δ 191.67, 165.44, 150.50, 140.54, 133.67, 132.44, 132.12, 131.79, 130.11, 126.22, 125.19, 122.49, 120.81, 120.78, 119.66, 119.62, 119.59, 110.91, 105.99, 97.20, 55.37, 46.22. LRMS calculated for C17H19ClNO⁺ [M+H]⁺, 344.1 found 344.1.

### Example 7

### 2-(4-nitrophenyl)thiophene

A mixture of 1-bromo-4-nitrobenzene (1.0 g, 50 mmol), thiophen-2-ylboronic acid (0.64 g, 5.0 mmol), Pd(PPh3)₄ (580 mg, 0.5 mmol), and Na2CO3 (1.1 g, 100 mmol) in dioxane (40 mL) and water (5 mL) was stirred at 90° C. under N₂ overnight. The reaction mixture was cooled to room temperature, and filtered over Celite. The filtrate was concentrated. The residue was dissolved in ethyl acetate (40 mL), and the solution was washed with brine, and dried over Na₂SO₄. The solution was filtered, and the filtrate was concentrated. The residue was purified by column chromatography (PE-PE/EA=10/1) to give 2-(4-nitrophenyl)thiophene (0.54 g, 52.6% yield). LRMS calculated for C10H8NO2S⁺ [M+H]⁺, 206.0 found 206.0.

### 4-(thiophen-2-yl)aniline

To a mixture of 2-(4-nitrophenyl)thiophene (540 mg, 2.63 mmol) in EtOH (30 mL) was added sat. NH₄Cl (5 mL), followed by iron powder (740 mg, 13.15 mmol). The resultant mixture was heated to reflux and stirred for 30 min, and then cooled to rt. The mixture was filtered over celite and the filtrate was concentrated. The residue was dissolved in EA. The solution was washed with brine, dried over Na₂SO₄, and filtered. The filtrate was concentrated. The residue was purified by column chromatography (PE/EA=4:1) to afford 4-(thiophen-2-yl)aniline (360 mg, 78.3% yield). 41 NMR (400 MHz, CDCl₃): δ 7.42-7.40 (d, J=8.4 Hz, 2H), 7.16-7.15 (m, 2H), 7.03-7.01 (m, 1H), 6.69-6.67 (d, J=8.4 Hz, 2H), 3.72 (br, 2H). LRMS calculated for C10H10NS⁺ [M+H]⁺, 176.1 found 176.1.

### 1-(2-((4-(thiophen-2-yl)phenyl)amino)phenyl)ethanone

A mixture of 4-(thiophen-2-yl)aniline (360 mg, 2.06 mmol), 1-(2-bromophenyl)ethanone (405 mg, 2.06 mmol), Pd₂(dba)₃ (190 mg, 0.21 mmol), xphos (150 mg, 0.32 mmol), and Cs₂CO₃ (1.30 g, 40 mmol) in dioxane (30 mL) was stirred at 90° C. under N₂ overnight. The reaction mixture was cooled to room temperature, and filtered over Celite. The filtrate was concentrated. The residue was dissolved in ethyl acetate (40 mL), and the solution was washed with brine, and dried over Na₂SO₄. The solution was filtered, and the filtrate was concentrated. The residue was purified by column chromatography (PE-PE/EA=8/1) to give 1-(2-((4-(thiophen-2-yl)phenyl)amino) phenyl)ethanone (130 mg, 21.5% yield). LRMS calculated for C18H16NOS⁺ [M+H]⁺, 294.1 found 294.1.

### 2-chloro-1-(2-((4-(thiophen-2-yl)phenyl)amino)phenyl)ethanone

To a mixture of 1-(2-((4-(thiophen-2-yl)phenyl)amino)phenyl)ethanone (130 mg, 0.44 mmol) in DCM (10 mL) were added DIEA (120 mg, 09 mmol) and TMSOTf (150 mg, 0.66 mmol) at 0° C. under N₂. The resulting mixture was warmed to rt and stirred for 2 h. NCS (70 mg, 0.53 mmol) was added and the mixture was stirred for another 2 h. It was quenched with water (10 mL), and the mixture was extracted with DCM (5 mL×3). The organic phase was washed with brine, dried over Na₂SO₄, and filtered. The filtrate was concentrated. The residue was purified by prep. TLC (PE/EA=5/1) to give the crude product (207 mg). The compound was further purified by reverse phase HPLC Gilson to afford the desired product as a yellow solid (54 mg, 35.2% yield); ¹H NMR (400 MHz, CDCl₃): δ 10.42 (s, 1H), 7.76-7.74 (d, J=8.0 Hz, 1H), 7.62-7.60 (d, J=8.4 Hz, 2H), 7.39-7.30 (m, 2H), 7.28-7.25 (m, 4H), 7.09-7.07 (dd, J=4.8 Hz, J=3.6 Hz, 1H), 6.79-6.75 (t, J=7.2 Hz, 1H), 4.76 (s, 2H); ¹³C NMR (100 MHz, CDCl₃): δ 193.05, 148.54, 143.93, 139.12, 135.52, 131.30, 130.60, 128.07, 126.97, 124.50, 123.41, 122.72, 117.00, 116.26, 114.86, 46.62. LRMS calculated for C18H15ClNOS⁺ [M+H]⁺, 328.1 found 328.1.

### Example 8

### 2-(3-nitrophenyl)thiophene

The method was same as compound 7, step 1 to give 2-(3-nitrophenyl)thiophene (650 mg, 63.1% yield); ¹H NMR (400 MHz, CDCl₃): δ 8.45 (s, 1H), 8.13-8.11 (d, J=8.4 Hz, 1H), 7.92-7.90 (d, J=7.6 Hz, 1H), 7.57-7.53 (t, J=8.0 Hz, 1H), 7.44 (d, J=3.6 Hz, 1H), 7.39 (d, J=4.8 Hz, 1H), 7.15-7.13 (m, 1H). LRMS calculated for C10H8NO2S⁺ [M+H]⁺, 206.0 found 206.0.

### 3-(thiophen-2-yl)aniline

The method was same as compound 7, step 2 to give 3-(thiophen-2-yl)aniline (440 mg, 78.1% yield). LRMS calculated for C10H10NS⁺ [M+H]⁺: 176.1, found 176.1.

### 1-(2-((3-(thiophen-2-yl)phenyl)amino)phenyl)ethanone

The method was same as compound 7, step 3 to give 1-(2-((3-(thiophen-2-yl)phenyl) amino)phenyl)ethanone (1.4 g, 78.1% yield). LRMS calculated for C18H16NOS⁺ [M+H]⁺: 294.1, found 294.1.

### 2-chloro-1-(2-((3-(thiophen-2-yl)phenyl)amino)phenyl)ethanone

The method was same as compound 7 to give 2-chloro-1-(2-((3-(thiophen-2-yl)phenyl)amino) phenyl)ethanone (27.4 mg, 61.6% yield); ¹H NMR (400 MHz, CDCl₃): δ 10.42 (s, 1H), 7.76-7.74 (d, J=8.0 Hz, 1H), 7.49 (s, 1H), 7.40-7.34 (m, 3H), 7.31-7.25 (m, 3H), 7.18-7.17 (d, J=7.2 Hz, 1H), 7.09-7.07 (m, 1H), 6.78-6.75 (m, 1H), 4.76 (s, 2H); ¹³C NMR (100 MHz, CDCl₃): δ 193.09, 148.77, 143.75, 40.36, 135.82, 135.56, 131.30, 129.97, 128.07, 125.51, 123.44, 122.31, 122.09, 116.95, 116.95, 116.20, 114.76, 46.62. LRMS calculated for C18H15ClNOS⁺ [M+H]⁺: 328.1, found 328.1.

### Example 9

### 5-(benzyloxy)pentan-1-ol

To a solution of pentane-1,5-diol (5.0 g, 48.01 mmol) in DMF (50 mL) was added 60% of NaH (1.3 g, 33.61 mmol) at 0° C. under N₂. The resulting mixture was warmed to room temperature and stirred for 30 min. BnBr (5.7 g, 33.61 mmol) was added at 0° C. The mixture was heated to 50° C. under N₂, and stirred for 30 min. The reaction mixture was cooled to room temperature, and it was quenched with water (100 mL) The mixture was extracted with EA (40 mL×3). The organic phase was washed with brine, dried over Na₂SO₄, and filtered. The filtrate was concentrated. The residue was purified by column chromatography (PE-PE/EA=2/1) to give 5-(benzyloxy)pentan-1-ol (2.2 g, 33.7%); ¹H NMR (400 MHz, CDCl₃): δ 7.26-7.34 (m, 5H), 4.50 (s, 2H), 3.64 (t, J=6.4 Hz, 2H), 3.48 (t, J=6.4 Hz, 2H), 1.56-1.67 (m, 4H), 1.41-1.49 (m, 2H), 1.32 (br, 1H).

### 5-(benzyloxy)pentyl Methanesulfonate

To a mixture of 5-(benzyloxy)pentan-1-ol (TED-549-1, 2.2 g, 11.32 mmol) in DCM (30 mL) were added MsCl (1.4 g, 12.45 mmol) and TEA (2.3 g, 22.64 mmol) at 0° C. under N₂. The resulting mixture was warmed to room temperature and stirred for 1 h. The reaction mixture was quenched with water (50 mL), and the mixture was extracted with DCM (30 mL×3). The organic phase was washed with brine, dried over Na₂SO₄, and filtered. The filtrate was concentrated to give 5-(benzyloxy)pentyl methanesulfonate (2.5 g, 81.3%); ¹H NMR (400 MHz, CDCl₃): δ 7.26-7.35 (m, 5H), 4.50 (s, 2H), 4.23 (t, J=6.4 Hz, 2H), 3.48 (t, J=6.4 Hz, 2H), 2.99 (s, 3H), 1.76-1.80 (m, 2H), 1.63-1.71 (m, 2H), 1.48-1.54 (m, 2H).

### 2-(2-((5-(benzyloxy)pentyl)oxy)ethoxy)ethanol

To a mixture of 2,2′-oxydiethanol (2.9 g, 27.53 mmol) in THF (50 mL) was added 60% of NaH (550 mg, 13.77 mmol) at 0° C. under N₂. The resulting mixture was warmed to room temperature and stirred for 30 min. A solution of 5-(benzyloxy)pentyl methanesulfonate (TED-549-2, 2.5 g, 9.18 mmol) in THF (10 mL) was added and the mixture was refluxed for 3 h under N₂, and then cooled to room temperature. The reaction mixture was quenched with water (150 mL), and the mixture was extracted with EA (40 mL×3). The organic phase was washed with brine, dried over Na₂SO₄, and filtered. The filtrate was concentrated. The residue was purified by column chromatography (PE/EA=10/1 to 1/1) to give 2-(2-((5-(benzyloxy)pentyl)oxy)ethoxy)ethanol (1.9 g, 73.4%); ¹H NMR (400 MHz, CDCl₃): δ 7.26-7.34 (m, 5H), 4.50 (s, 2H), 3.71-3.74 (m, 2H), 3.66-3.69 (m, 2H), 3.57-3.63 (m, 4H), 3.47 (t, J=6.4 Hz, 4H), 2.47 (br, 1H), 1.59-1.68 (m, 4H), 1.40-1.47 (m, 2H).

### 2-(2-(45-(benzyloxy)pentyl)oxy)ethoxy)ethyl Methanesulfonate

To a mixture of 2-(2-((5-(benzyloxy)pentyl)oxy)ethoxy)ethanol (1.9 g, 6.74 mmol) in DCM (50 mL) were added MsCl (0.92 g, 8.09 mmol) and TEA (1.4 g, 13.48 mmol) at 0° C. under N₂. The resulting mixture was warmed to room temperature and stirred for 1 h. The reaction mixture was quenched with water (100 mL), and the mixture was extracted with DCM (30 mL×3). The organic phase was washed with brine, dried over Na₂SO₄, and filtered. The filtrate was concentrated to give 2-(2-((5-(benzyloxy)pentyl)oxy)ethoxy)ethyl methanesulfonate (1.8 g, 74.1%); ¹H NMR (400 MHz, CDCl₃): δ 7.26-7.37 (m, 5H), 4.50 (s, 2H), 4.38 (t, J=4.4 Hz, 2H), 3.76 (t, J=4.4 Hz, 2H), 3.64-3.66 (m, 2H), 3.56-3.58 (m, 2H), 3.43-3.49 (m, 4H), 3.06 (s, 3H), 1.57-1.65 (m, 4H), 1.38-1.46 (m, 2H).

### N,N-diBoc-2-(2-((5-(benzyloxy)pentyl)oxy)ethoxy)ethan-1-amine

A mixture of 2-(2-((5-(benzyloxy)pentyl)oxy)ethoxy)ethyl methanesulfonate (1.8 g, 5.00 mmol), HNBoc₂ (1.2 g, 5.5 mmol) and K₂CO₃ (1.4 g, 10.00 mmol) in DMF (30 mL) was heated to 100° C. under N₂ and the mixture was stirred for 3 h. It was then cooled to room temperature. The reaction mixture was quenched with water (100 mL), and extracted with EA (40 mL×3). The organic phase was washed with brine, dried over Na₂SO₄, and filtered. The filtrate was concentrated. The residue was purified by column chromatography (PE/EA=10/1 to 3/1) to give N,N-diBoc-2-(2-((5-(benzyloxy)pentyl)oxy)ethoxy)ethan-1-amine (2.0 g, 83.1%); ¹HNMR (400 MHz, CDCl₃): δ 7.26-7.34 (m, 5H), 4.50 (s, 2H), 3.74-3.80 (m, 2H), 3.53-3.63 (m, 6H), 3.42-3.48 (m, 4H), 1.58-1.65 (m, 4H), 1.49 (s, 18H), 1.39-1.45 (m, 2H).

### N-(2-(2-((5-hydroxypentyl)oxy)ethoxy)ethyl)-5-(2-oxohexahydro-1H-thieno[3,4-d]imidazol-4-yl)pentanamide

A mixture of ditert-butyl (2-(2-((5-(benzyloxy)pentyl)oxy)ethoxy)ethyl)carbamate (2.0 g, 4.15 mmol) and 10% of Pd/C (300 mg) in MeOH (100 mL) was stirred for 18 h under H2. The reaction mixture was filtered through Celite and the filtrate was concentrated. The residue was dissolved in DCM (10 mL) and 6M HCl in dioxane (5 mL) was added. The resulting mixture was stirred at room temperature for 2 h and then the solvent was removed. The residue was dissolved in DMF (15 mL), and TEA (1.03 g, 10.02 mmol) was added, followed by HATU (1.16 g, 3.06 mmol). The resulting mixture was stirred at room temperature for 2 h, and concentrated. The crude product was purified by prep-HPLC to give N-(2-(2-((5-hydroxypentyl)oxy)ethoxy)ethyl)-5-(2-oxohexahydro-1H-thieno[3,4-d]imidazol-4-yl)pentanamide (120 mg, 6.9%); ¹H NMR (400 MHz, CD₃OD): δ 4.47-4.50 (m, 1H), 4.29-4.32 (m, 1H), 3.48-3.59 (m, 10H), 3.36 (t, J=5.6 Hz, 2H), 3.18-3.23 (m, 1H), 2.90-2.95 (m, 1H), 2.70 (t, J=12.8 Hz, 1H), 2.21 (t, J=7.2 Hz, 2H), 1.54-1.75 (m, 8H), 1.40-1.48 (m, 4H). LRMS calculated for C19H36N3O5S⁺ [M+H]⁺, 418.6, found 418.2.

### Tert-butyldimethyl(3-nitrophenoxy)silane

To a solution of 3-nitrophenol (1.0 g, 7.20 mmol) in DCM (20 mL) were added TBSC1 (1.2 g, 7.92 mmol) and imidazole (979 mg, 14.40 mmol) at room temperature. The resulting mixture was stirred for 2 h and quenched with water (40 mL) It was extracted with DCM (30 mL×3). The organic phase was washed with brine, dried over Na₂SO₄, and filtered. The filtrate was concentrated. The residue was purified by column chromatography (PE-PE/EA=2/1) to give tert-butyldimethyl(3-nitrophenoxy)silane (1.3 g, 71.4%); ¹H NMR (400 MHz, CDCl₃): δ 7.83 (d, J=8.0 Hz, 1H), 7.66 (t, J=2.4 Hz, 1H), 7.38 (t, J=8.0 Hz, 1H), 7.16 (d, J=8.0 Hz, 1H), 1.00 (s, 9H), 0.24 (s, 6H), 1.41-1.49 (m, 2H), 1.32 (br, 1H).

### 3-((tert-butyldimethylsilyl)oxy)aniline

To a mixture of tert-butyldimethyl(3-nitrophenoxy)silane (1.3 g, 5.14 mmol) in

MeOH (20 mL) was added 10% of Pd/C (200 mg). The resulting mixture was stirred at room temperature overnight under H₂ (1 atm). The reaction mixture was filtered through Celite and the filtrate was concentrated to give 3-((tert-butyldimethylsilyl)oxy)aniline (1.1 g, 95.7%); LRMS calculated for C12H22NOSi⁺ [M+H]⁺, 224.4, found 224.3.

### 1-(2-((3-((cert-butyldimethyl silyl)oxy)phenyl)amino)phenyl)ethan-1-one

The mixture of 3-((tert-butyldimethylsilyl)oxy)aniline 1-(2-bromophenyl)ethanone (562 mg, 2.83 mmol), Pd₂(dba)₃ (246 mg, 0.27 mmol), xphos (257 mg, 0.54 mmol), and Cs₂CO₃ (1.30 g, 4.04 mmol) in dioxane (15 mL) was heated to 90° C. under N₂, and it was stirred for 2 h. The reaction mixture was cooled to room temperature, filtered through Celite and the filtrate was concentrated. The residue was dissolved in EA (40 mL), washed with brine, and dried over Na₂SO₄. It was filtered, and the filtrate was concentrated. The residue was purified by column chromatography (PE-PE/EA=20/1) to give 1-(2-((3-((tert-butyldimethylsilyl)oxy) phenyl)amino)phenyl)ethan-1-one (410 mg, 44.6%); 1H NMR (400 MHz, CDCl₃): δ 10.47 (s, 1H), 7.81 (d, J=8.4 Hz, 1H), 7.28-7.33 (m, 2H), 7.18 (t, J=8.0 Hz, 1H), 6.85 (d, J=7.6 Hz, 1H), 6.71-6.74 (m, 2H), 6.61 (d, J=8.4 Hz, 1H), 2.64 (s, 3H), 0.98 (s, 9H), 0.21 (s, 6H).

### 1-(2-((3-hydroxyphenyl)amino)phenyl)ethan-1-one

To a solution of 1-(2-((3-((cert-butyldimethylsilyl)oxy)phenyl)amino)phenyl)ethan-1-one (100 mg, 0.29 mmol) in THF (2 mL) was added 1M TBAF in THF (0.35 mL, 0.35 mmol) dropwise at room temperature. The resulting mixture was stirred for 1 h and quenched with water (20 mL) The mixture was extracted with EA (10 mL×3), and the organic phase was washed with brine, and dried over Na₂SO₄. It was filtered, and the filtrate was concentrated. The residue was purified by prep-TLC (DCM/MeOH=10/1) to give 1-(2-((3-hydroxyphenyl) amino)phenyl)ethan-1-one (52 mg, 70.8%); ¹H NMR (400 MHz, CDCl₃): δ 10.49 (s, 1H), 7.82 (d, J=8.0 Hz, 1H), 7.32 (d, J=7.6 Hz, 2H), 7.20 (t, J=8.0 Hz, 1H), 6.83 (d, J=8.0 Hz, 1H), 6.73-6.77 (m, 2H), 6.58 (d, J=8.0 Hz, 1H), 2.65 (s, 3H). LRMS calculated for C14H14NO⁺ [M+H]⁺, 228.3 found 228.1.

### N-(2-(2-((5-(3-((2-acetylphenyl)amino)phenoxy)pentyl)oxy)ethoxy)ethyl)-5-(2-oxohexahydro-1H-thieno[3,4-d]imidazol-4-yl)pentanamide

To a mixture of N-(2-(2-((5-hydroxypentyl)oxy)ethoxy)ethyl)-5-(2-oxohexahydro-1H-thieno[3,4-d]imidazol-4-yl)pentanamide (50 mg, 0.12 mmol), 1-(2-((3-hydroxyphenyl)amino)phenyl)ethan-1-one (35 mg, 0.16 mmol), and PPh₃ (63 mg, 0.24 mmol) in dioxane (3 mL) was added DIAD (53 mg, 0.26 mmol) at 0° C. under N₂. The resulting mixture was warmed to room temperature and stirred overnight. The reaction mixture was quenched with water (5 mL), and extracted with EA (10 mL×2). The organic phase was washed with brine, dried over Na₂SO₄, and filtered. The filtrate was concentrated. The residue was purified by prep-TLC (DCM/MeOH=5/1) to give N-(2-(2-((5-(3-((2-acetylphenyl)amino)phenoxy)pentyl)oxy)ethoxy)ethyl)-5-(2-oxohexahydro-1H-thieno[3,4-d]imidazol-4-yl)pentanamide (42 mg, 46% yield); ¹H NMR (400 MHz, CDCl₃): δ 10.50 (s, 1H), 7.81 (d, J=8.0 Hz, 1H), 7.26-7.34 (m, 2H), 7.12-7.24 (m, 1H), 6.72-6.85 (m, 2H), 6.63-6.66 (m, 1H), 6.48-6.58 (m, 1H), 6.12 (s, 1H), 5.22 (s, 1H), 4.86-4.97 (m, 1H), 4.46-4.49 (m, 1H), 4.28-4.31 (m, 1H), 3.88-3.96 (m, 2H), 3.44-3.64 (m, 10H), 3.10-3.14 (m, 1H), 2.87-2.91 (m, 1H), 2.70-2.74 (m, 1H), 2.64 (s, 2H), 2.15-2.26 (m, 2H), 1.56-1.84 (m, 12H). LRMS calculated for C33H47N4O6S⁺ [M+H]⁺, 627.3 found 627.4.

### N-(2-(2-((5-(3-((2-(2-chloroacetyl)phenyl)amino)phenoxy)pentyl)oxy)ethoxy)ethyl)-5-(2-oxohexahydro-1H-thieno[3,4-d]imidazol-4-yl)pentanamide

The method was the same as compound 4 to give N-(2-(2-((5-(3-((2-(2-chloroacetyl) phenyl)amino)phenoxy)pentyl)oxy)ethoxy)ethyl)-5-(2-oxohexahydro-1H-thieno[3,4-d]imidazol-4-yl)pentanamide (11 mg); ¹H NMR (400 MHz, CDCl₃): δ 10.33 (s, 1H), 7.71-7.80 (m, 2H), 6.72-6.85 (m, 4H), 6.60-6.62 (m, 3H), 6.30-6.39 (m, 2H), 5.44 (s, 2H), 4.88-5.09 (m, 3H), 4.76 (s, 2H), 4.70 (s, 1H), 4.47 (s, 2H), 4.29 (m, 2H), 3.92-4.00 (m, 5H), 3.43-3.60 (m, 25H), 3.05-3.15 (m, 2H), 2.86-2.91 (m, 2H), 2.65-2.74 (m, 3H), 2.21 (br, 6H). LRMS calculated for C33H46ClN4O6S⁺ [M+H]⁺, 661.3, found 661.2.

### Example 10

Example 10 was made according to procedures outlined herein. As tested described herein, Example 10 showed minimal inhibition in the EC₅₀ assay.

### Example 11

Example 11 was made according to procedures outlined herein. As tested described herein, Example 11 provided an EC₅₀ of 11.7 μM with a max of 50%.

### Example 12

Example 12 was made according to procedures outlined herein. As tested described herein, Example 12 provided an EC₅₀ of 13.7 μM with a max of 23%.

In Silico Protein Preparation

The crystal structures of TEAD4.YAP (PDB ID: 3JUA), TEAD2.PLM (PDB ID: SHGU, palmitic acid), and TEAD2.FLF (PDB ID: 5DQ8, flufenamic acid, compound 1) were retrieved and prepared using the Protein Preparation Wizard in the Schrödinger software package (Schrödinger LLC, New York, N.Y., 2017). Bond orders were assigned and hydrogen atoms were added. Missing side chains and loops were introduced using the Prime module. The resulting protein and compound structures were protonated at pH 7.0 using PROPKA and Epik, respectively. The structure of compound 2 was generated by replacing the acetic acid on FLF with chloromethyl ketone. Subsequently, the binding modes of PLP and compound 2 to TEAD4 were obtained using the align function in PyMOL.

Covalent Docking

The covalent structure of TEAD4.compound 2 was generated using CovDock. The chloromethyl ketone group of compound 2 was defined as the reaction group for a nucleophilic substitution reaction with the TEAD4 Cys-360. Residues within 3.0 Å of compound 2 were refined during covalent docking. The covalent bond parameters from the OPLS force field were extracted.

Molecular Dynamics Simulations

The structures of TEAD4.Yap1, TEAD4.Yap1.PLM, non-covalent [TEAD4.compound 2].Yap1, and covalent [TEAD4-compound 2].Yap1 were used to run molecular dynamics simulations using an AMBER14 software package. The restrained electrostatic potential (RESP) atomic charges of PLM, compound 2 in the covalent and non-covalent complexes were calculated at the HF/6-31G*level using the Gaussian 09 package. In the covalent [TEAD4-compound 2].Yap1 complex, compound 2, Leu-359, Cys-360, and Glu-361 were extracted for RESP charge fitting. The atom charges of Cys-360 were replaced by RESP charges and the optimized parameters of bond length, bond angle, and dihedral angle between Cys-360 and compound 2 were used to build new frcmod parameters. The α-carbon atom of compound 2 and sulfur atom of Cys-360 were bonded using tleap program.

Complexes were immersed in a box of TIP3P water molecules. No atom on the complex was within 14 Å of any side of the box. The solvated box was further neutralized with Na⁺ or Cl⁻ counterions using the tleap program. Simulations were carried out using the GPU accelerated version of the pmemd program with ff14SB and gaff force fields in periodic boundary conditions. All bonds involving hydrogen atoms were constrained by using the SHAKE algorithm, and a 2 femtoseconds (fs) time step was used in the simulation. The particle mesh Ewald (PME) method was used to treat long-range electrostatics. Simulations were run at 298 K under 1 atm in NPT ensemble employing Langevin thermostat and Berendsen barostat. Water molecules were first energy-minimized and equilibrated by running a short simulation with the complex fixed using Cartesian restraints. A series of energy minimizations were subsequently applied in which the Cartesian restraints were gradually relaxed from 500 kcal·Å⁻² to 0 kcal·Å⁻², and the system was subsequently gradually heated to 298 K with a 48 μs molecular dynamics run. For each complex, 50 independent simulations (replicates) were generated that were each 50 ns in length. The initial velocity of each replicate was randomly assigned. In total, 2.5 μs of simulation was run for each complex.

Free Energy Calculations

In each of the 50 trajectories (50 ns in length), the first 2 ns were discarded for equilibration. Snapshots were saved every 1 ps, yielding 48000 structures per trajectory. 30000 snapshots were selected at regular intervals for free energy calculations using the cpptraj program. The Molecular Mechanics-Generalized Born Surface Area (MM-GBSA) method was used to calculate the free energy using the MMPBSA.py script. The calculation using the GB method was performed with sander and Onufriev's GB model. Solvent-accessible surface area (SASA) calculations were switched to the icosahedron (ICOSA) method, where surface areas were computed by recursively approximating a sphere around an atom, starting from an icosahedron. Salt concentration was set to 0.1 M. The entropy was determined by normal mode calculations with the mmpbsa_py_nabnmode module by selecting 150 of the 30000 snapshots used in the free energy calculations at regular intervals. The maximum number of cycles of minimization was set to 10000. The convergence criterion for the energy gradient to stop minimization was 0.5. In total, 30000 frames were used for each MM-GBSA calculations while 150 frames were used for each normal mode analysis. All other parameters were left at default values.

The MM-GBSA binding free energy is expressed as:

ΔG_{MM-GBSA}=ΔE_{GBTOT}−TΔS_{NMODE}

where ΔE_{GBTOT} is the combined internal and solvation energies, T is the temperature (298.15 K). ΔS_{NMODE} is the entropy determined by normal mode calculations. The total enthalpy from the generalized Born model, ΔE_{GBTOT}, is the sum of 4 components:

ΔE_{GBTOT}=ΔE_{VDW}+ΔE_{ELE}+ΔE_{GB}+ΔE_{SURF}

where ΔE_{VDW} and ΔE_{ELE} are the van der Waals and electrostatic energies, respectively, and ΔE_{GB} and ΔE_{SUPF} are the polar and non-polar desolvation energies, respectively. All binding energies are determined by:

ΔE=E^{COM}−E^{REC}−E^{LIG}

where E^{COM}, E^{REC} and E^{LIG} are total energies corresponding to the complex, receptor, and ligand, respectively. The relative difference in free energy is determined by:

ΔΔG=ΔG_{COM}−ΔG_{APO}

where ΔG_{COM} and ΔG_{APO} are the covalent or non-covalent complex and the unbound native apo complex, respectively.

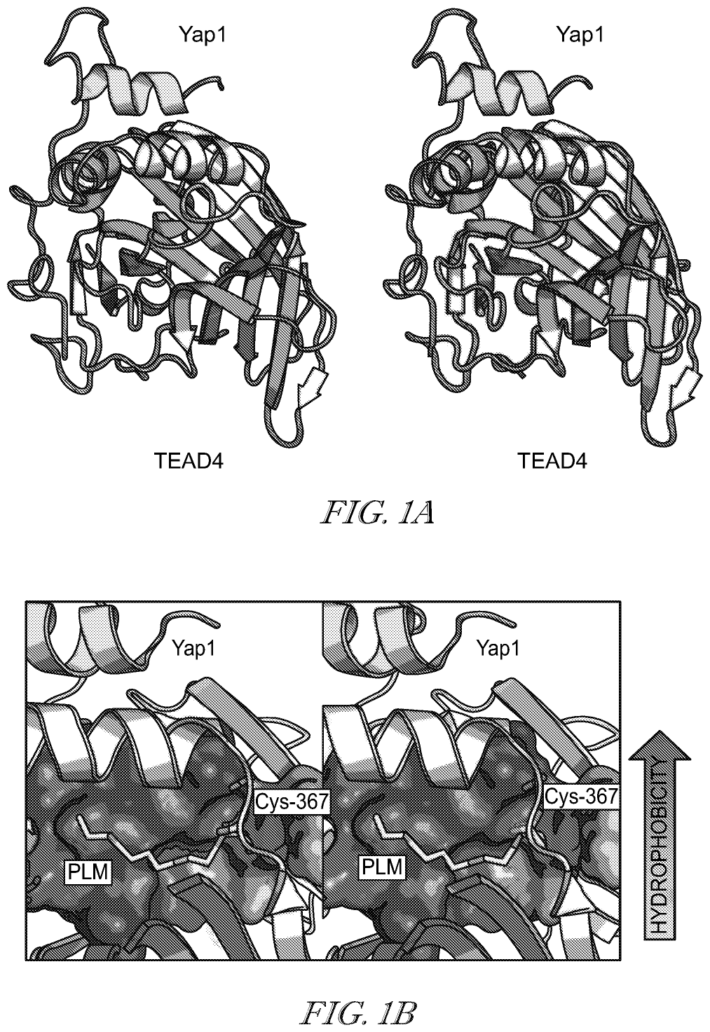

Covalent bond formation at allosteric pocket cysteine reduces TEAD4.Yap1 affinity. As shown in FIG. 1A, the TEAD three-dimensional structure contains a 12-strand β-sandwich fold, flanked by four short α-helices and the N-terminal region of Yap1 (residues 61-100) forms an α-helix (residues 61-73), which binds between TEAD α3 and α4 helices, and an Ω-loop (residues 85-99), which binds near TEAD α1 and β12. As shown in FIG. 1B, crystal structures of TEADs reveal the presence of a deep hydrophobic pocket that is occupied by palmitate. Flufenamic acid (compound 1) binds weakly to two sites on TEAD2, but it did not inhibit TEAD binding to Yap. One of the binding sites is located within the deep hydrophobic palmitate-binding pocket of the transcription factor and the other at the protein-protein interaction interface. The binding mode of compound 1 in the deep pocket of TEAD2 shows that that the carboxylic acid moiety of compound 1 is located near the thiol of a conserved cysteine residue (Cys-367) that is the acylation site of a palmitoyl group.

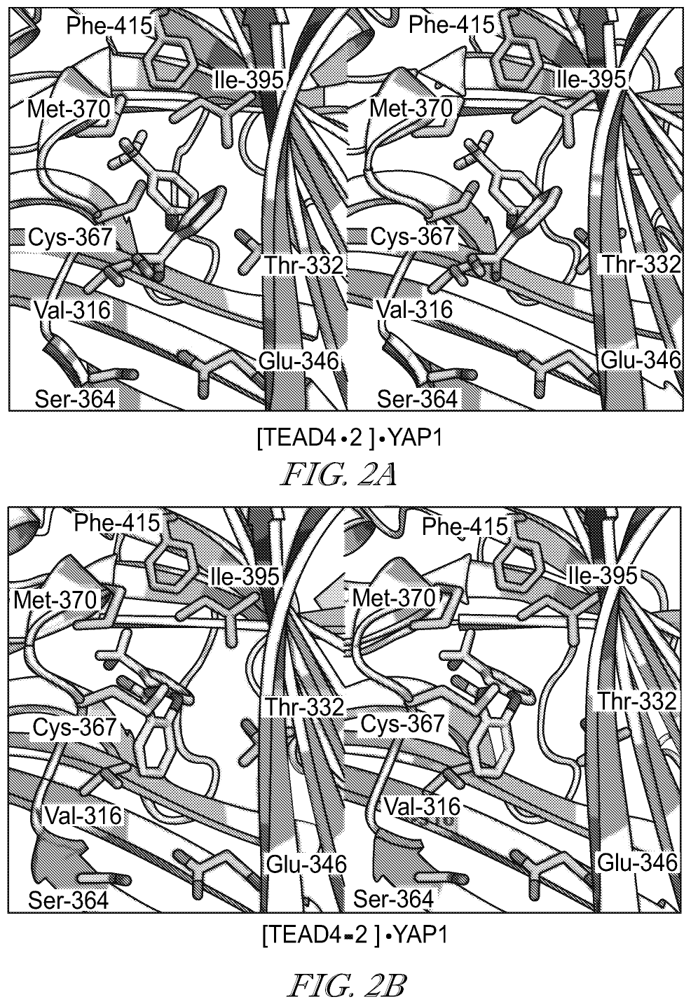

Compound 2 was synthesized with a chloromethyl ketone moiety that can form a covalent bond with Cys-367. Microsecond explicit-solvent molecular dynamics simulations were applied to determine whether covalent bond formation at the cysteine residue affects TEAD4.Yap1 protein-protein interaction. Three separate simulations were carried out: TEAD4.Yap1; [TEAD4.compound 2].Yap1 non-covalent complex (FIG. 2A) and [TEAD4-compound 2].Yap1 covalent complex (FIG. 2B). Each simulation consisted of 50 separate 50-ns trajectories resulting in 2.5 μs (50×50 ns) of explicit-solvent molecular dynamics simulations per complex. Structures sampled from these simulations were collected to determine the free energy of binding of TEAD4 to Yap1 in each of the complexes using the widely-used MM-GBSA free energy calculation method. The results are shown in Table 3. Non-covalent binding of compound 2 to TEAD4 exhibited little change to the TEAD4.Yap1 binding affinity (ΔΔG_{MMPBSA}=0.5±0.1 kcal/mol). However, covalent adduct formation of compound 2 to TEAD4 led to substantially greater loss of TEAD4 affinity to Yap1 by nearly 10.9±0.1 kcal/mol. The 20-fold reduction in the binding affinity suggested that adduct formation at Cys-367 leads to allosteric inhibition of the TEAD4.Yap1 protein-protein interaction. These results were confirmed by repeating the calculations with compound 5. Non-covalent binding of the compound led to little change for the affinity of the TEAD4.Yap1 complex (ΔΔG_{MMGBSA}=−0.5±0.1 kcal/mol), while covalent bond formation led to substantial reduction in the MM-GBSA binding affinity to 5.3±0.1 kcal/mol. These results demonstrate that mere binding to the pocket is insufficient to disrupt the protein-protein interaction, whereas covalent bond formation with the cysteine residue may lead to inhibition of the interaction. The crystal structures have been deposited in Protein Data Bank (http://www.rcsb.org/pdb) for TEAD2 in complex with compound 2.

Table 1 shows the free energy calculations between the change in free energy of the non-covalently and covalent bound compounds with the apo TEAD4.Yap1 complex; mean±s.e.; n=30000 snapshots.

TABLE 1
Energy Exchange Between Covalent and Non-Covalent Bound Compounds

| | | ΔΔE_{VDW} + | ΔΔE_{ELE} + | | |
|---|---|---|---|---|---|
| State | Complex | ΔΔE_{SURF} | ΔΔE_{GB} | Δ(TΔS) | ΔΔG |
| Non-covalent | [TEAD4.2].Yap1 | −3.1 ± 0.1 | 1.3 ± 0.7 | 1.9 ± 0.1 | 0.5 |
| Covalent | [TEAD4.2].Yap1 | −1.6 ± 0.1 | 12.2 ± 0.7 | 0.1 ± 0.1 | 10.9 |
| Non-covalent | [TEAD4.5].Yap1 | −1.0 ± 0.1 | 0.9 ± 0.7 | −1.0 ± 0.1 | −0.5 |
| Covalent | [TEAD4.5].Yap1 | −6.3 ± 0.1 | 9.8 ± 0.7 | 0.6 ± 0.1 | 5.3 |

FIGS. 1A-1B show three-dimensional structures and free energy calculations. FIG. 1A illustrates a stereo view of the X-ray structure of the TEAD4.Yap1 complex (PDB ID: 3JUA). TEAD4 and Yap1 are shown in ribbon representation. FIG. 1B illustrates the structure of the TEAD4.Yap1 complex depicting the deep hydrophobic pocket of TEAD4. The pocket is occupied by palmitate, which is shown as capped-sticks. The pocket is shown in solvent-accessible surface area with varying levels of hydrophobicity. FIG. 2A illustrates a non-covalent complex of compound 2 bound to TEAD4. Compound 2 and surrounding amino acids are shown as capped sticks. FIG. 2B illustrates a covalent complex of compound 2 and TEAD4. Compound 2 and surrounding residues are shown as capped-sticks.

Protein Expression and Purification

TEAD4 (217-434), TEAD4 (217-434) Cys367Ser mutant and Yap1 (Full-length) were expressed as GST-fusion proteins in BL-21 (DE3) strain of E. coli from the pGEX-6P-1 vector. Transformed bacteria were grown in LB at 37° C. until they reached an OD₆₀₀ of 0.6-0.8. Isopropyl-β-D-galactoside (IPTG) was added to a final concentration of 0.5 mM and cells were then incubated at 16° C. for 16 h. Cell pellets were re-suspended in a buffer containing 200 mM NaCl, 20 mM Tris, 2 mM dithiothreitol (DTT), pH 8.0, and lysed by passage through a microfluidizer. Cell debris was removed by centrifugation at 35,000×g for 1 h. Clarified lysates were loaded onto a pre-equilibrated 5 mL GSTrap HP column at 1 ml/min. The column was washed with 10 column volumes of buffer and the protein was eluted with 10 mM reduced glutathione in the same buffer. The protein was further purified on a HiLoad 26/600 Superdex 200 pg SEC column (GE, Boston, Mass.) with 100 mM NaCl, 20 mM Tris, 2 mM DTT, pH 8.0 as buffer. The GST-tag was cleaved from proteins by incubation with the HRV-3C protease (Sigma-Aldrich, St. Louis, Mo.) at 100:1 w/w ratio while dialyzing against PBS with 5 mM (3-mercaptoethanol for 48 h at 4° C. The cleavage solution was passed through a GSTrap FF column to remove the cleaved GST and the HRV-3C enzyme. Cleavage was verified by SDS-PAGE and mass spectrometry.

TEAD2 (217-447) was expressed as N-terminal HIS-fusion protein in BL-21 (DE3) strain of E. coli from the pET-28a vector. Transformed bacteria were grown in Terrific Broth at 37° C. until they reached an OD₆₀₀ of 0.6-0.8. IPTG was added to a final concentration of 0.5 mM and cells were then incubated at 16° C. for 16 h. Cell pellets were re-suspended in a buffer containing 500 mM NaCl, 50 mM HEPES, 8 mM β-mercaptoethanol, pH 7.5 and lysed by multiple passages through a microfluidizer. Cell debris was removed by centrifugation at 35,000×g for 1 h. Clarified lysates were loaded onto a pre-equilibrated 5 mL HisTrap FF column at 1 mL/min. The column was washed with 100 mL of buffer containing 300 mM NaCl, 25 mM HEPES, 1 mM TCEP, 5% v/v glycerol, 30 mM imidazole, pH 7.5 prior to elution with the same buffer containing 500 mM imidazole. The protein was further purified on a HiLoad 26/600 Superdex 200 pg SEC column (GE, Boston, Mass.) with 150 mM NaCl, 25 mM HEPES, 1 mM TCEP, pH 7.5 as buffer. For crystallization trials, the elution from the HisTrapFF affinity chromatography was dialyzed against 150 mM NaCl, 50 mM Tris pH 8.0 for 2 h, then cleaved with 1:100 w/w thrombin at 4° C. overnight. The cleaved protein was dialyzed against 300 mM NaCl, 25 mM HEPES, 1 mM TCEP, 5% v/v glycerol, 10 mM imidazole, pH 7.5. The cleaved HIS-tag was removed by passing through the HisTrap FF column. TEAD2 without the HIS-tag was further purified on SEC, as above.

Size-Exclusion Chromatography

2 ml of 6.3 μM GST-TEAD4 in PBS was incubated with 100 μM 2 compound 2 in 2% v/v DMSO or DMSO without compound for 24 h at 4° C. After the incubation, the samples were injected into a HiLoad 26/600 Superdex 200 pg SEC column, pre-equilibrated with PBS. The elution profile of the column was analyzed for protein aggregation.

Fluorescence Polarization

GST-TEAD4, GST-TEAD4 Cys367Ser mutant or HIS-TEAD2 interaction with Yap1 was investigated using a fluorescently-labeled peptide (FAM-Yap₆₀₋₉₉), consisting of FAM-labeled TEAD-binding peptide fragment of Yap1 (FAM-DSETDLEALFNAVMNPKTANVPQ TVPMCLRKLPASFCKPP), which has a disulfide bridge (American Peptide, Sunnyvale, Calif.). Addition of FAM-Yap₆₀₋₉₉ to the TEAD was followed by measurement of changes in polarization. 40 μL of 125 nM GST-TEAD4 WT or GST-TEAD4 Cys367Ser in assay buffer (PBS with 0.01% v/v Triton-X100) or 40 μL of 64 nM HIS-TEAD2 was added to a 384-well black polystyrene plate (Cat. No. 262260; Nunc, Roskilde, Denmark) and incubated with 5 μL of 22000 μM serially diluted compounds in assay buffer supplemented with 20% v/v DMSO for 24 h at 4° C. Finally, 5 μL of 160 nM FAM-Yap₆₀₋₉₉ peptide was added, the plate centrifuged, and the polarization was measured on an Envision Multilabel Plate Reader (PerkinElmer, Waltham, Mass.) using a filter set with excitation and emission wavelengths of 485 and 535 nm, respectively. Percent inhibition was calculated as relative to a minimum inhibition control, which is without compound, and a maximum inhibition control, which is without a TEAD.

For the determination of the inhibition efficiency k_{inact}/K₁, the protein compound incubation time was varied between 0.548 h, prior to the addition of the FAM-Yap₆₀₋₉₉ peptide and fluorescence polarization measurements. The progressive decrease in TEAD activities were plotted against time for all 10 concentrations (0.2100 μM) of the compounds and the observed rate of inhibition (k_{obs}) was calculated by fitting a simple exponential function.

The observed rate of inhibition was then plotted against the concentration of the compound and a polynomial function k_{obs}=k_{inact}[Inhibitor]/K₁+[Inhibitor] was fitted to determine the k_{inact} and K₁ values.

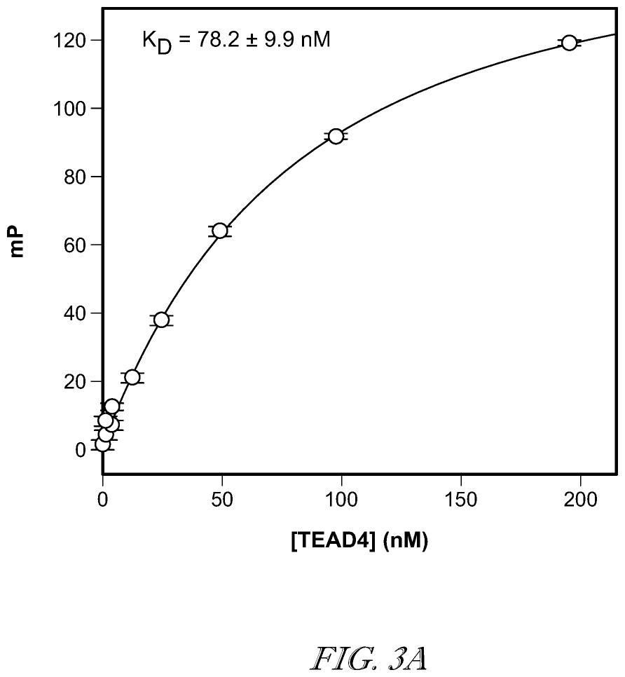

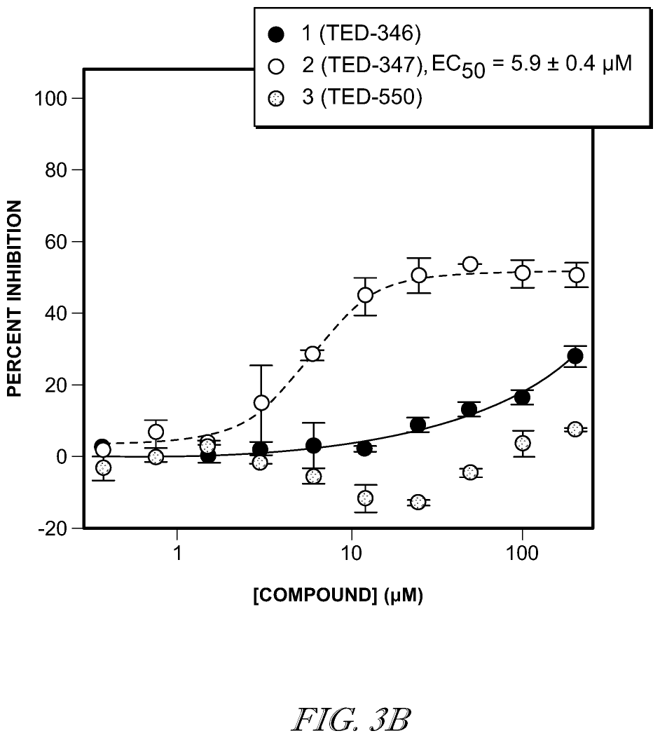

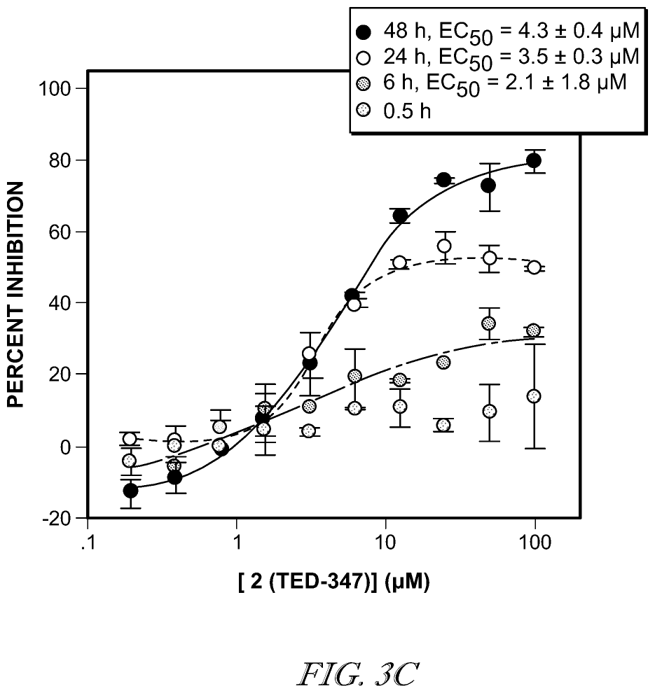

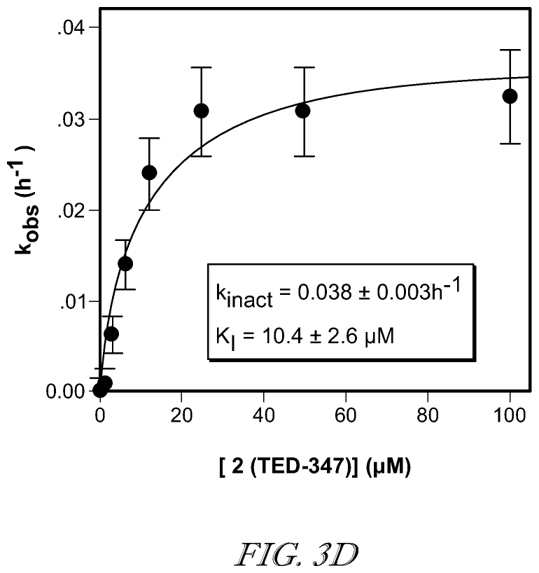

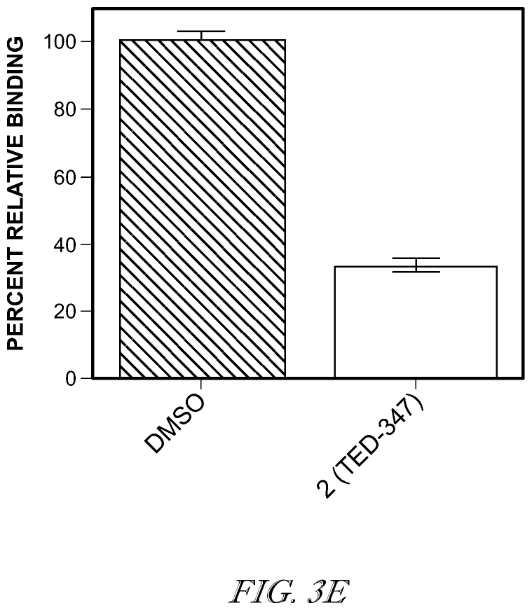

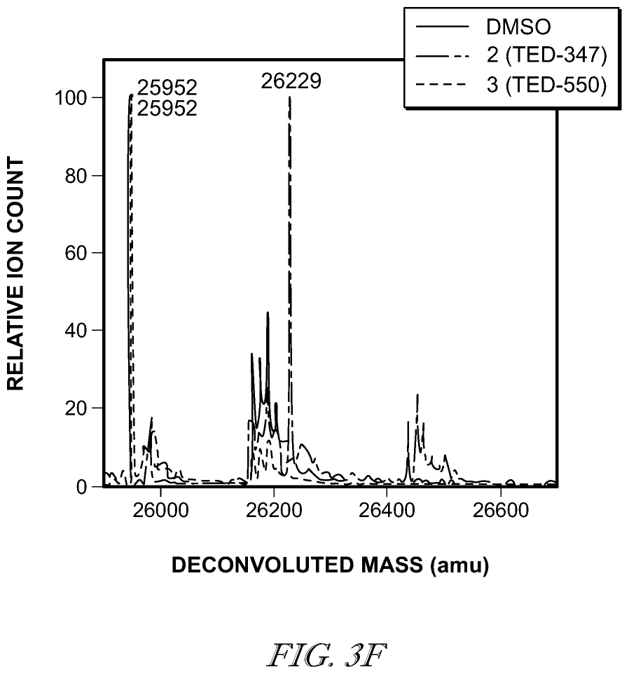

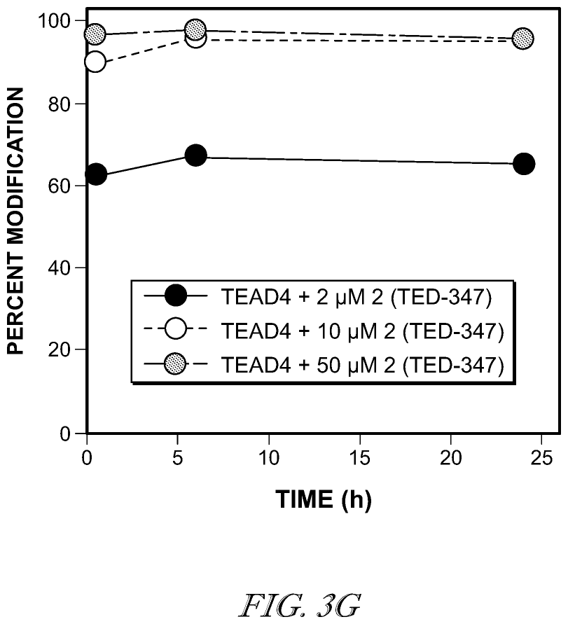

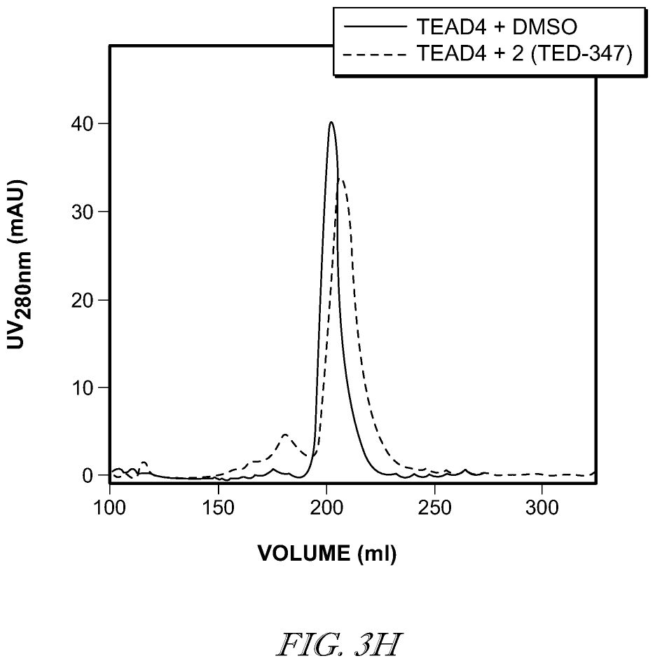

Compound 2 and derivatives form covalent adducts at an allosteric site and inhibit TEAD4 binding to Yap1. Compound 2 was prepared to determine whether it formed a covalent complex with TEAD4. FAM-YAP₆₀₋₉₉ includes the entire Yap1.TEAD4 binding interface. The labeled peptide binds to TEAD4 with a K_{D} of 78.2±9.9 nM. Compound 1 was tested and it was found that compound 1 did not inhibit the TEAD4.Yap1 interaction, consistent with previous studies as shown in FIG. 3B. The effects of other compounds on the TEAD4.Yap1 interaction were also tested using the fluorescence polarization assay. Following 24 h incubation of TEAD4 with compound 2 at 4° C., it was found that compound 2 inhibited the TEAD4.Yap1 protein-protein interaction by 53% with an apparent EC₅₀ of 5.9±0.4 μM. Compound 3 did not inhibit, as compound 3 cannot form a covalent adduct since the chlorine atom is replaced by a methyl group (FIG. 3B). As shown in FIG. 3C, a time-dependent study was performed at 0.5, 6, 24 and 48 h for compound 2, where compound 2 reached maximum inhibition of 80% at 48 h. Based on the time and concentration-dependent inhibition study of TEAD4 with compound 2 (FIG. 3D), the rates of inactivation were calculated for the compounds and several derivatives (Table 1). Table 1 shows the change in free energy between non-covalently and covalent bound compounds; mean±s.e.; n=30000 snapshots. P values were calculated using two-tailed t-tests. ***P<0.0005. The maximum rate of inactivation of compound 2 was calculated to be 0.038±0.003 h-′ (FIG. 3D), corresponding to a t_{1/2}^{∞} of 18.2 h. To determine whether compound 2 is a reversible or irreversible inhibitor, TEAD4 was incubated with 50 μM compound for 24 h at 4° C., and then dialyzed against buffer for 24 h at 4° C., prior to interaction with the fluorescently labeled Yap1 peptide (FIG. 3E). Compound 2 inhibited the TEAD4.Yap1 interaction, even after dialysis, indicating that compound 2 is an irreversible inhibitor.

Protein Mass Spectrometry

The samples were centrifuged at 20,000×g for 20 mM to remove precipitants before being injected into an empty column on an Agilent 1200 liquid chromatography system (Agilent, Santa Clara, Calif.), using 80% Buffer A (H₂O, 5 mM NH₄OAc) and 20% Buffer B (ACN, 5 mM NH₄OAc), and the masses were detected on an Agilent 6520 Accurate Mass Q-TOF.

It was determined whether a covalent bond formed between inventive compounds and TEAD4. Following incubation of TEAD4 at 10 μM with 200 μM of compound 2 for 24 h at 4° C., a peak at 26229 was observed, corresponding to the TEAD4.compound 2 adduct, while the peak at 25952 corresponding to TEAD4 disappeared (FIG. 3F). Compound 3 only showed a peak at 25952 indicating no adduct formation. The covalent adduct formation by compound 2 was relatively fast (FIG. 3G), reaching nearly 100% adduct formation after 30 min incubation with TEAD4. Because the rate of inhibition developed over a longer timescale (FIG. 3C), compound 2 is proposed to induce a slow conformational change in TEAD4 that prevents its interaction with Yap1. To rule out the possibility that compound 2 induces slow aggregation of TEAD4, and not the proposed slow conformational change, GST-TEAD4 was incubated with DMSO compound 2 for 24 h at 4° C., followed by injection into an SEC column (FIG. 3H). No significant aggregation of GST-TEAD4 was observed after 24 h incubation, with or without compound 2. A slight increase in dimer formation for the TEAD4 sample incubated with compound 2 occurred compared to the sample incubated with DMSO. In addition, a slight shift in the retention time of the TEAD4 sample incubated with compound 2, as well as peak broadening, compared to the TEAD4 incubated with DMSO, both of which suggest conformational change of the protein. Furthermore, it is highly unlikely that Cys-367 oxidation was responsible for the lack of 100% inhibition at longer times since it has been shown that covalent bond formation is rapid and complete within less than an hour. Also, whole protein mass spectrometry carried out at 24 h did not reveal the presence of the oxidized species.

FIG. 3A illustrates increasing concentration of TEAD4 incubated with 16 nM FAM-labeled Yap (FAM-Yap₆₀₋₉₉) peptide followed by measurements of changes in fluorescence polarization (mean±s.d.; n=3). FIG. 3B illustrates measured changes in fluorescence polarization when TEAD4 was incubated with increasing concentration of compound for 24 h at 4° C. followed by addition of FAM-Yap₆₀₋₉₉ (mean±s.d.; n=3). FIG. 3C illustrates assessment of time-dependent inhibition of TEAD4 by compound 2 by fluorescence polarization using FAM-Yap₆₀₋₉₉ at 10 different concentration (0.1-100 μM) following 0.5, 6, 24 and 48 h incubation at 4° C. (mean±s.d.; n=3). FIG. 3D illustrates assessment of time-dependent inhibition of TEAD4 by compound 2 by fluorescence polarization at 10 concentrations ranging from 0.1 to 100 μM following incubation at 0.5, 6, 24, and 48 h at 4° C. (mean±s.d.; n=3). The observed rate of inactivation (k_{obs}) was calculated at each compound concentration using percent inhibition data at each time point. The rate constant is plotted against the concentration of compound. FIG. 3E illustrates fluorescence polarization measurements when TEAD4 was incubated with 50 μM of compound 2 for 24 h at 4° C., then dialyzed against PBS for 24 h at 4° C., prior to addition of FAM-Yap₆₀₋₉₉ (mean±s.d.; n=3). FIG. 3F illustrates analysis by ESI mass spectrometry when 10 μM of TEAD4 was incubated with 200 μM compounds for 24 h at 4° C. FIG. 3G illustrates analysis by ESI mass spectrometry when 10 μM TEAD4 was incubated with 2, 10, 50 μM compound 2 for 0.5, 6, 25 h at 4° C. Percent ratio of the adduct over total protein signal, quantified from the relative ion count, is plotted versus time. FIG. 3H illustrates that no significant aggregation is observed when TEAD4 is incubated with DMSO or compound 2 followed by injection into SEC column.

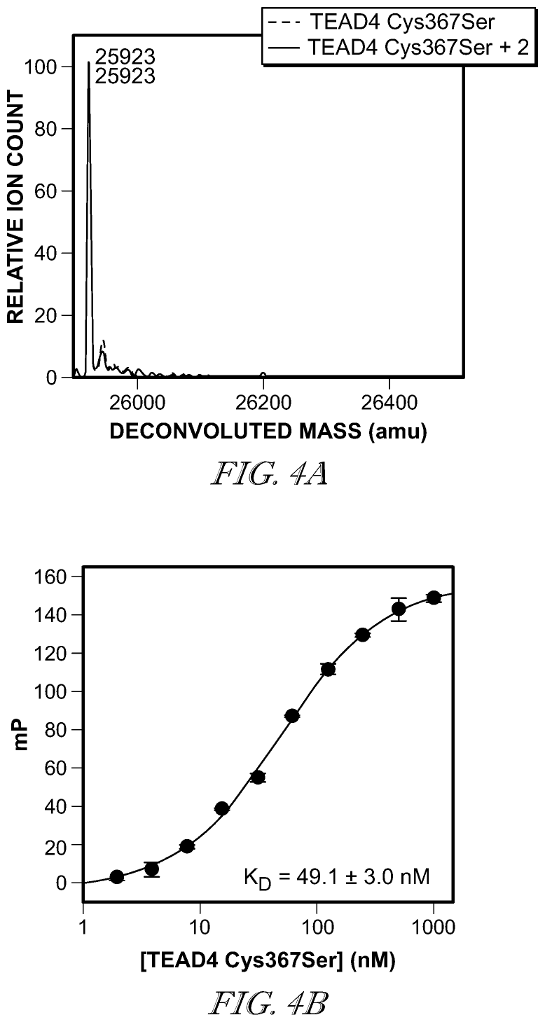

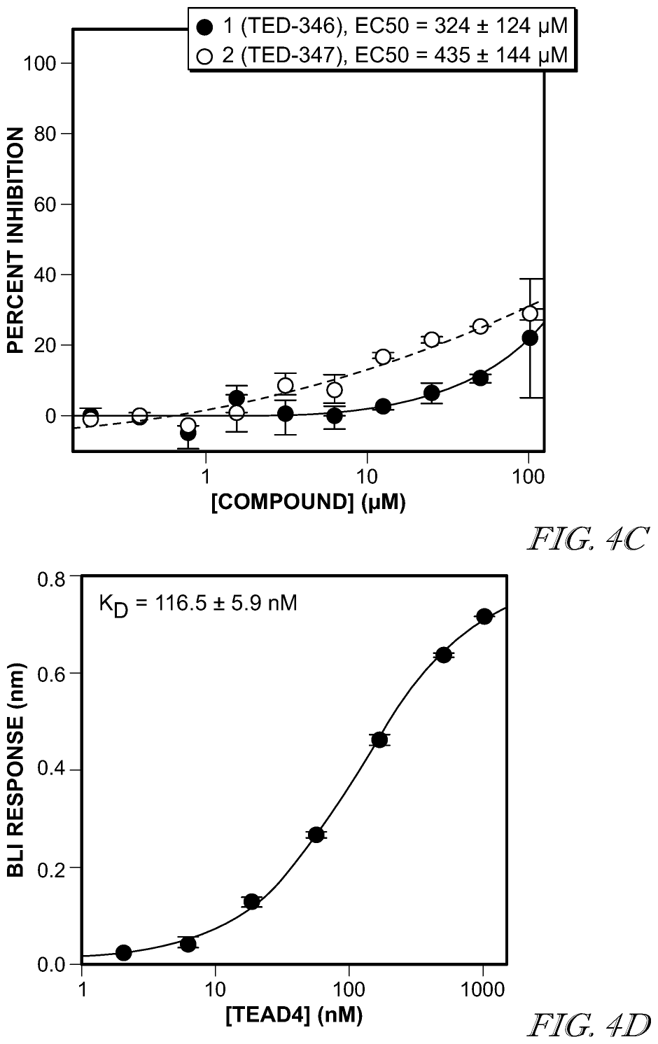

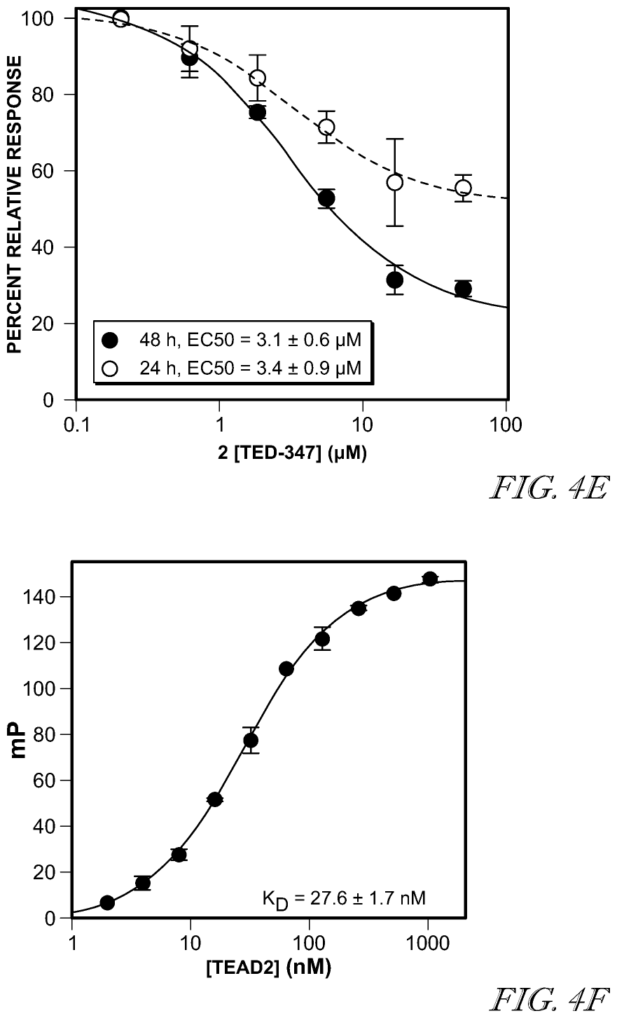

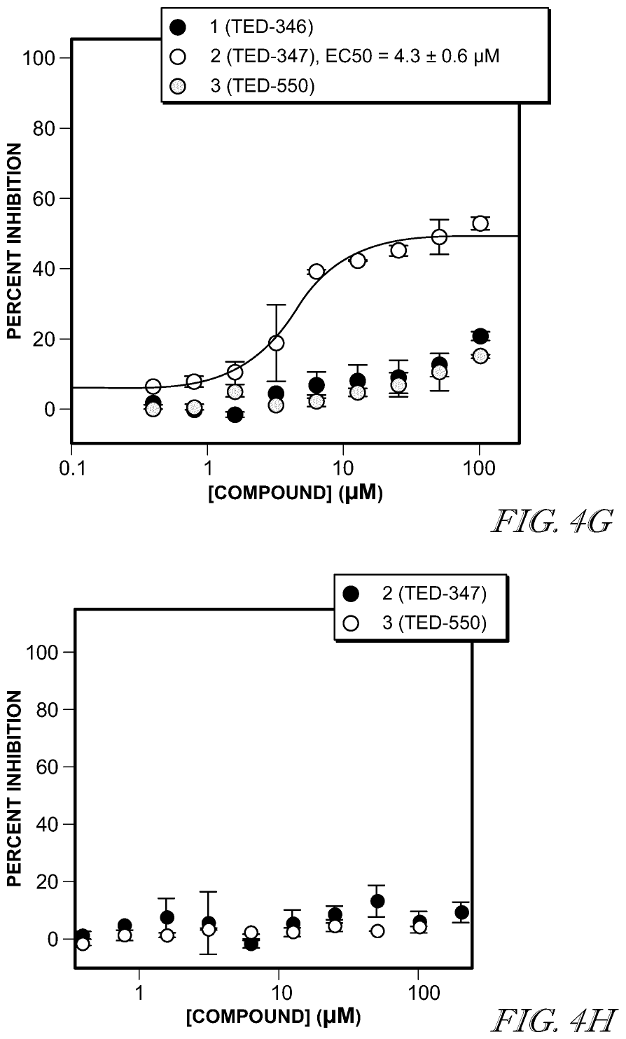

To further establish that compound 2 specifically forms a bond with Cys-367, within the central pocket of TEAD4, its interaction was tested with a TEAD4 Cys367Ser mutant. Adduct formation by compound 2 to the mutant TEAD4 Cys367Ser was analyzed by mass spectrometry. After 24 h, compound 2 failed to form an adduct with the mutant protein (FIG. 4A). TEAD4 Cys367Ser mutant showed no change in affinity for FAM-YAP₆₀₋₉₉ peptide, with a K_{D} of 49.1±3.0 nM (FIG. 4B). Subsequently, the compounds were tested for inhibition of the peptide binding to TEAD4. Compound 2 did not inhibit the mutant TEAD4 Cys367Ser protein binding to the peptide, suggesting that covalent adduct formation by compound 2 is essential for its ability to inhibit the protein-protein interaction (FIG. 4C).

FIG. 4A illustrates analysis by ESI mass spectrometry after 10 μM TEAD4 Cys367Ser mutant was incubated with 200 μM compound for 24 h at 4° C. FIG. 4B illustrates measurement of fluorescence polarization due to binding when increasing concentration of TEAD4 Cys367Ser mutant was mixed with 16 nM FAM-Yap₆₀₋₉₉ peptide (mean±s.d.; n=3). FIG. 4C illustrates measurement of fluorescence polarization due to binding when TEAD4 Cys367Ser mutant was incubated with increasing concentration of compounds for 24 h at 4° C. followed by addition of FAM-Yap₆₀₋₉₉ (mean±s.d.; n=3).

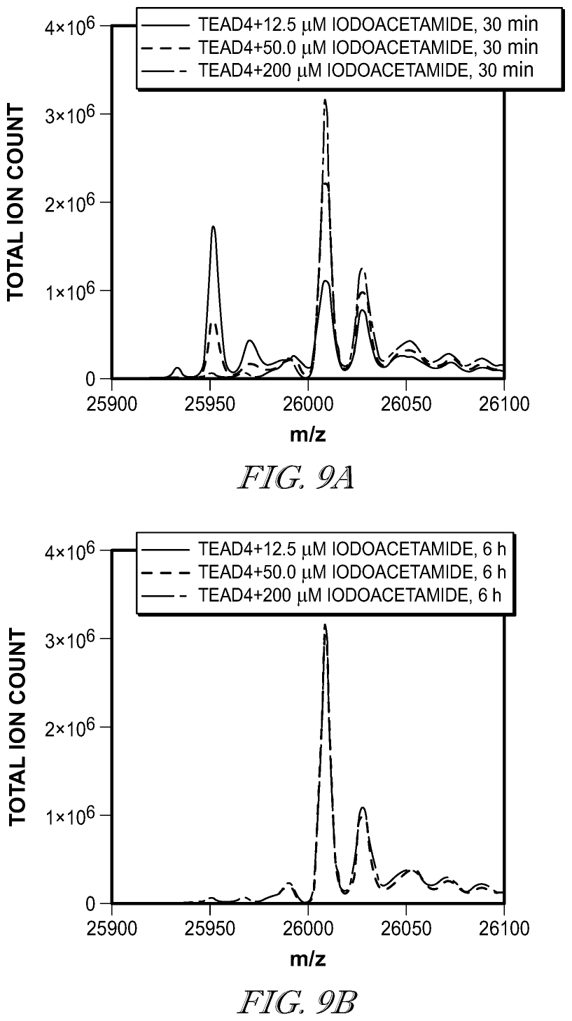

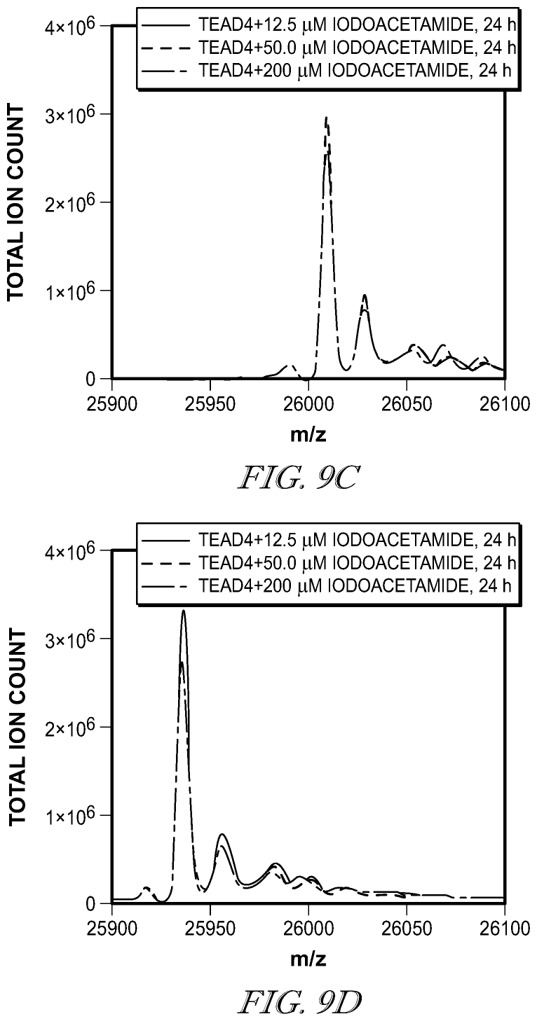

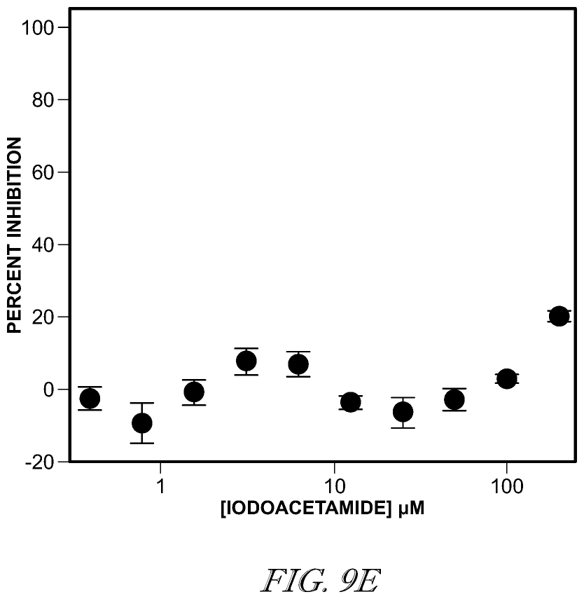

ESI mass spectrometry was used to detect formation of adducts by iodoacetamide. In <30 minutes, concentration-dependent adduct formation up to 200 μM was observed, where the protein was modified by a single adduct (FIG. 9A). After 6 h, the protein was modified by a single adduct at all concentrations of iodoacetamide (FIG. 9B). Presence of a second reaction site was not observed until 24 h at the highest tested concentration of 200 μM (FIG. 9C). To determine whether Cys-367 is the target of the single adduct, TEAD4 Cys367Ser mutant was reacted with varying concentrations of iodoacetamide for 24 h. After 24 h, there was no modification of the protein, except for a small adduct that was detected only at 200 μM iodoacetamide concentration, which is consistent with the wild-type TEAD4 (FIG. 9D). Although iodoacetamide was able to react with TEAD4 Cys-367, it was unable to inhibit the activity of the protein in the FP assay (FIG. 9E). Thus, merely reacting with the cysteine to form a covalent bond with conserved Cys-367 does not guarantee inhibition of activity from the TEAD4.Yap1 protein-protein interaction.

FIGS. 9A-9E illustrates that TEAD4 Cys-367 is reactive to iodoacetamide. A sample of 5 μM TEAD4 was reacted with 12.5, 50, and 200 μM iodoacetamide at 4° C. for FIG. 9A) 30 min, for FIG. 9B) 6 h, and for FIG. 9C) 24 h. Wild-type TEAD4 YBD construct showed an apparent MW of 25952, and the iodoacetamide adduct was +57. FIG 9D) A sample of 5 μM TEAD4 Cys367Ser mutant was reacted with 12.5, 50, and 200 μM iodoacetamide at 4° C. for 24 h. TEAD4 YBD Cys367Ser mutant construct showed an apparent MW of 25936. FIG. 9E) TEAD4 was incubated with increasing concentration of iodoacetamide for 24 h at 4° C. followed by addition of FAM-Yap₆₀₋₉₉ to measure changes in fluorescence polarization.

Biolayer Interferometry

Biolayer interferometry was measured on OctetRed 384 (ForteBio, Menlo Park, Calif.) using PBS with 0.025% v/v Tween-20 at 30° C. with constant shaking at 1000 rpm. Streptavidin-conjugated sensors (ForteBio, Menlo Park, Calif.) were loaded with 30 μg/ml biotin-labeled GST-Yap or biocytin and were introduced to 1-1000 nM TEAD4. The sensors were regenerated with 5 mM HCl solution after each interaction. For compound inhibition study, 100 nM TEAD4 was pre-incubated with 0.1100 μM compound 2 in 2% v/v DMSO for 24 h at 4° C. before interaction with captured GST-Yap.

To further confirm whether compound 2 inhibited the interaction between TEAD4 and Yap1 peptide, biolayer interferometry (BLI) that used full-length Yap1 protein was applied. The binding affinity between GST-tagged Yap1 and the TEAD4 protein was found to be 116.5±5.9 nM (FIG. 4D), which was comparable to that of the FAM-YAP₆₀₋₉₉ peptide. TEAD4 was incubated with compound 2 for 24 or 48 h at 4° C. before studying its interaction with GST-Yap1 using BLI. As with the FP assay, dose- and time-dependent inhibition of TEAD4 binding to full-length Yap1 (FIG. 4E) was observed.

Since the palmitate-binding pocket and the Cys-367 residue is conserved in all 4 human TEAD proteins, whether compound 2 would be active against TEAD2 was tested. His-tagged TEAD2 protein was tested in the FP binding assay, where it showed an apparent K_{d} of 27.6±1.7 nM (FIG. 4F). Compounds 1-3 were incubated with TEAD2 for 24 h at 4° C. before the addition of the Yap1 peptide, and compound 2 was shown to inhibit TEAD2, while compound 1 and compound 3 were inactive (FIG. 4G). It is expected that compound 2 will likely inhibit protein-protein interactions of TEAD1 and TEAD4 with Yap1 considering their close structural similarity to TEAD4.

The selectivity of the compounds was explored with two unrelated protein-protein interactions between (i) the urokinase receptor (uPAR) and its ligand urokinase (uPA); and (ii) the a subunit of the voltage-gated calcium channel Cav2.2 with its β subunit Cavβ₃. Using previously developed fluorescence polarization assays for these interactions, it was found that compound 2 and compound 3 showed no inhibition of uPAR.uPA or Cav2.2 α·β protein-protein interactions (FIG. 4H and FIG. 4I). Both proteins have cysteine residues capable of forming covalent adducts. These results further confirm the selectivity of compound 2.

FIG. 4D illustrates BLI measurements of the binding of TEAD4 to captured Yap when biotin-labeled GST-Yap1 was captured onto streptavidin-conjugated biolayer interferometry sensors which were dipped into varying concentrations of TEAD4. (mean±s.d.; n=3). FIG. 4E illustrates BLI measurements when biotin-labeled GST-Yap1 was captured onto streptavidin-conjugated biolayer interferometry sensors which were dipped into solutions containing 100 nM TEAD4, pre-incubated with varying concentrations of compound 2 for 24 or 48 h at 4° C. (mean±s.d.; n=3). FIG. 4F illustrates measured fluorescence polarization when increasing concentration of HIS-TEAD2 was mixed with 16 nM FAM-Yap₆₀₋₉₉ peptide (mean±s.d.; n=3). FIG. 4G illustrates measured fluorescence polarization when HIS-TEAD2 was incubated with increasing concentration of compounds for 24 h at 4° C. followed by addition of FAM-Yap₆₀₋₉₉ (mean±s.d.; n=3). FIG. 4H illustrates measured fluorescence polarization when urokinase receptor (uPAR) was incubated with varying concentrations of compounds for 24 h at 4° C. followed by addition of a urokinase-derived fluorescently-labeled peptide AE147 (mean±s.d.; n=3). FIG. 4I illustrates measured fluorescence polarization when the β-3 subunit of the voltage-gated calcium channel Cav2.2 was incubated with varying concentrations of compounds for 24 h at 4° C. followed by addition of an α-subunit peptide that was fluorescently labeled (mean±s.d.; n=3).

Crystallization of TEAD2 and Structure Refinement

Purified TEAD2 was concentrated to 12 mg/mL and crystallized at 20° C. using the hanging-drop vapor-diffusion method with a reservoir solution containing 0.1 M HEPES (pH 7.27.4) and 2.4-2.8 M sodium formate. The crystals were soaked in reservoir solution supplemented with 3-5 mM of compound 2 and 25% v/v glycerol for 3 h and were subsequently flash-cooled in liquid nitrogen. To rule out the possibility that the observed density of compound 2 was not the endogenous S-palmitoylation from protein expression, some crystals were soaked in a cryo-protectant solution supplemented with 2 mM DTT for 2 h to soak out the fatty acid. The crystal structure of these crystals was solved and no extra electron-density was observed. Another batch of crystals were soaked in three steps: 1)—in a cryo-protectant solution supplemented with 2 mM DTT for 2 h, 2)—in a cryo-protectant solution (wash) for 2 h and 3)—in a cryo-protectant solution supplemented with 3-5 mM of compound 2 for 3 h.

Data was collected at beamline 4.2.2 at the Advanced Light Source (ALS, Berkeley, Calif., USA) and processed with XDS. All crystals contained two molecules per asymmetric unit and the symmetry corresponded to space group C₂. Molecular Replacement was used to obtain the initial phases using Phaser and the crystal structure of TEAD2 transcriptional activation domain (PDB 5EMV) as the search model. Initial model building was carried out using Autobuild in PHENIX. The final model (Rfree 0.268, with good geometry and no Ramachandran outliers) was obtained by iterative cycles of manual building in Coot and refinements with PHENIX-refine.

A TEAD2.compound 2 complex was formed by soaking TEAD2 crystals with compound 2. The crystal diffracted to 2.43 Å resolution, and the structure was solved in space group C2 with two TEAD2 per asymmetric unit (Table 2).

TABLE 2
X-ray crystallographic data-collection and refinement
statistics of TEAD2 covalently bound to compound 2.

TEAD2 covalently bound to
compound 2

Data collection

| | |
|---|---|
| Wavelength (Å) | 0.97625 |
| Space group | C2 |

Cell dimensions

| | | | |
|---|---|---|---|
| a, b, c (Å) | 123.76 | 61.23 | 79.84 |
| α, β, γ(°) | 90.00 | 116.89 | 90.00 |

| | |
|---|---|
| Resolution (Å) | 48.17-2.43 |
| R_{sym} | 0.098 (1.198) |
| Rₘₑₐₛ | 0.116 (1.423) |
| Rₚᵢₘ | 0.061 (0.760) |
| CC1/2 | 0.996 (0.601) |
| I/σ(I) | 9.8 (1.0) |
| Completeness (%) | 99.7 (100.0) |
| Multiplicity | 3.6 (3.5) |

Refinement

| | |
|---|---|
| Resolution (Å) | 38.89-2.43 (2.56-2.43) |
| No. unique reflections | 20132 |
| R_{work} | 0.2205 |
| R_{free} | 0.2678 |

RMS deviations

| | |
|---|---|
| Bond lengths (Å) | 0.003 |
| Bond angles (°) | 0.601 |

No. atoms

| | |
|---|---|
| Protein | 3400 |
| Ligand | 52 |

B-factors (Å²)

| | |
|---|---|
| Protein/ligand/ions | 61.82 |

Ramachandran plot

| | |
|---|---|
| Favored (%) | 96.3 |
| Allowed (%) | 3.2 |
| MolProbity score | 1.21 |
| Rotamer outliers (%) | 0.27 |

*Highest-resolution shell values are shown in parentheses.

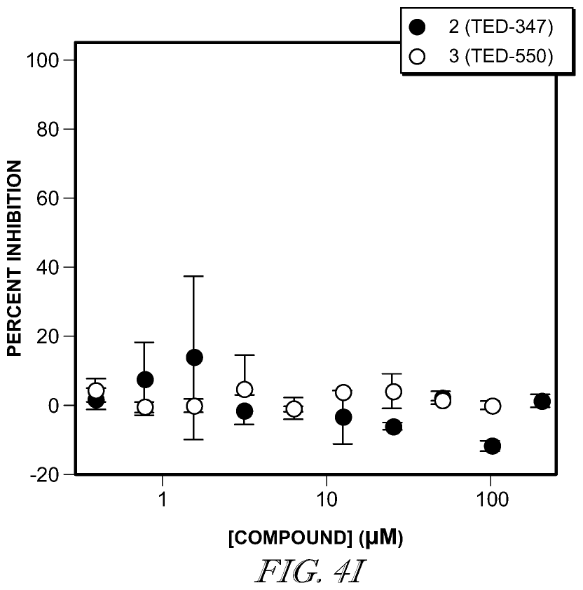

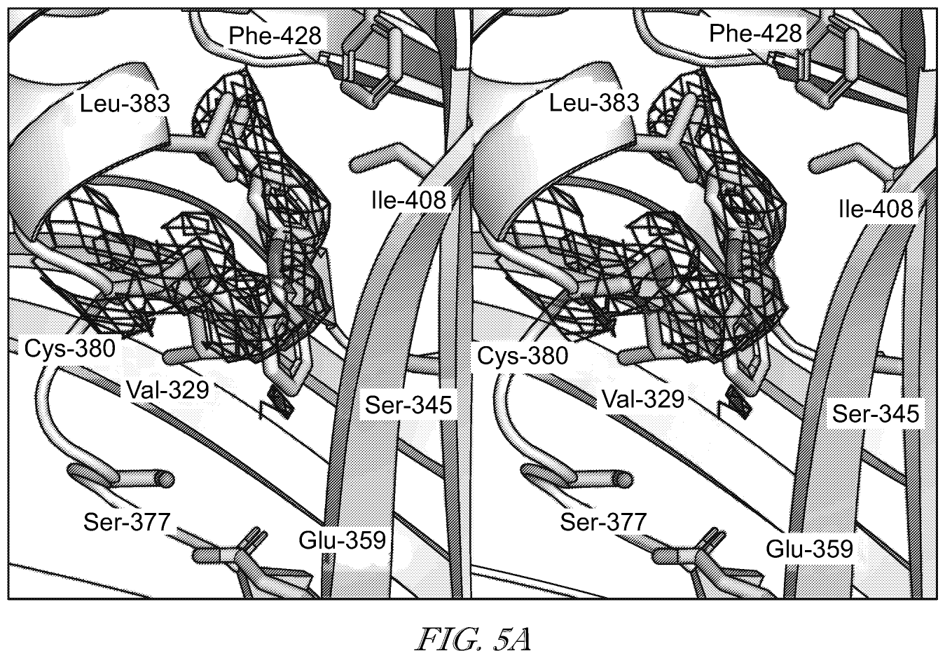

The overall structure of TEAD2 in complex with compound 2 was the same as previously published structures, with a Cα RMSD of 0.59 Å, compared to a previously published structure (PDB ID: 5DQ8). The density of compound 2 within the central binding site is weak (FIG. 5A), possibly indicating less than 100% occupancy. To confirm that the observed density is compound 2 and not palmitate, the fatty acid was soaked out by incubating the crystal in a buffer containing DTT for 2 h. No density within the central pocket was present after this treatment. A 3-step soaking experiment was performed, where the crystal was first soaked in buffer containing DTT for 2 h to remove the fatty acid, then exchanged into buffer without DTT for 2 h, and finally incubated with compound 2 for 3-4 h. The crystal quality suffered after the treatment, but an unambiguous positive density was observed in the pocket, covalently attached to Cys-380. Compared to the structure of TEAD2 in complex with compound 1 (PDB ID: 5DQ8), the first benzene ring of compound 2 is rotated away from the direction of Val-347 by about 90° to allow the covalent bond to form. The second ring and the trifluoromethyl group is shifted further into the hydrophobic pocket (FIGS. 5A and B). Compound 2 forms a covalent bonded interaction with TEAD2 at the proposed site of Cys-380.

FIGS. SA-SB illustrate the crystal structure of TEAD2 in complex with compound 2. FIG. 5A illustrates a stereo image of compound 2 covalently bound to Cys-380 in the central binding pocket of TEAD2. The 2|Fo|−|Fc|α_{calc} map around compound 2 is illustrated in black mesh. Compound 2 and residues near the reaction site of compound 2 are shown in sticks with accompanying labels. FIG. 5B depicts a two-dimensional ligand interaction map of covalently bound compound 2 in the central pocket of TEAD2.

Biochemical Studies of Compound 2 Derivatives

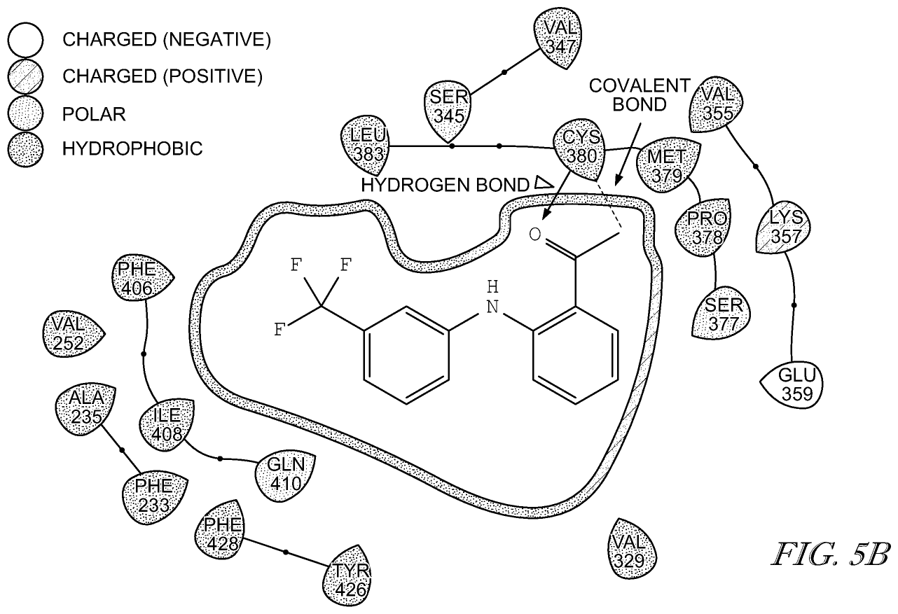

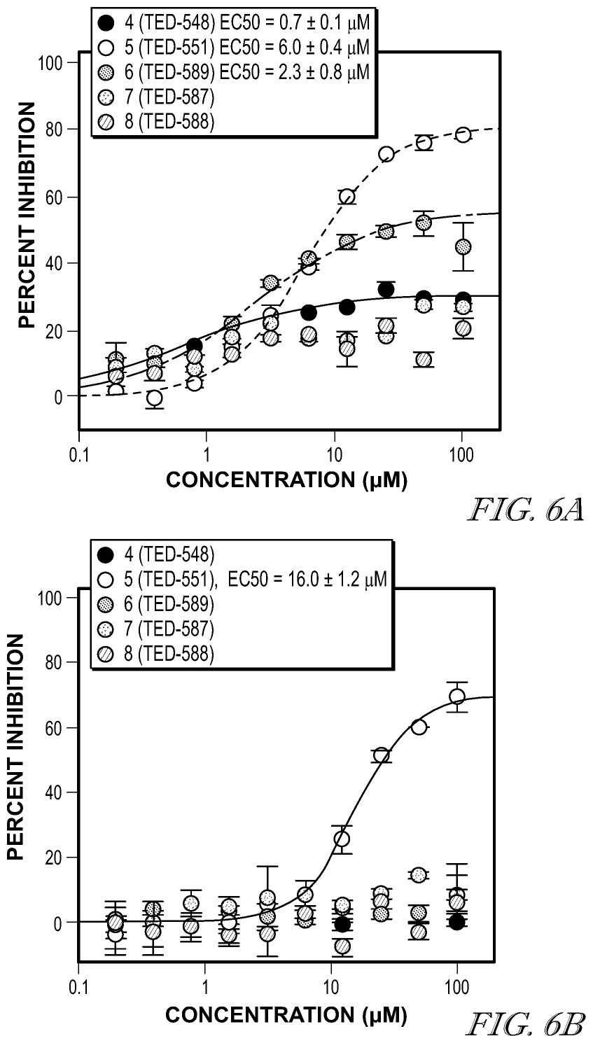

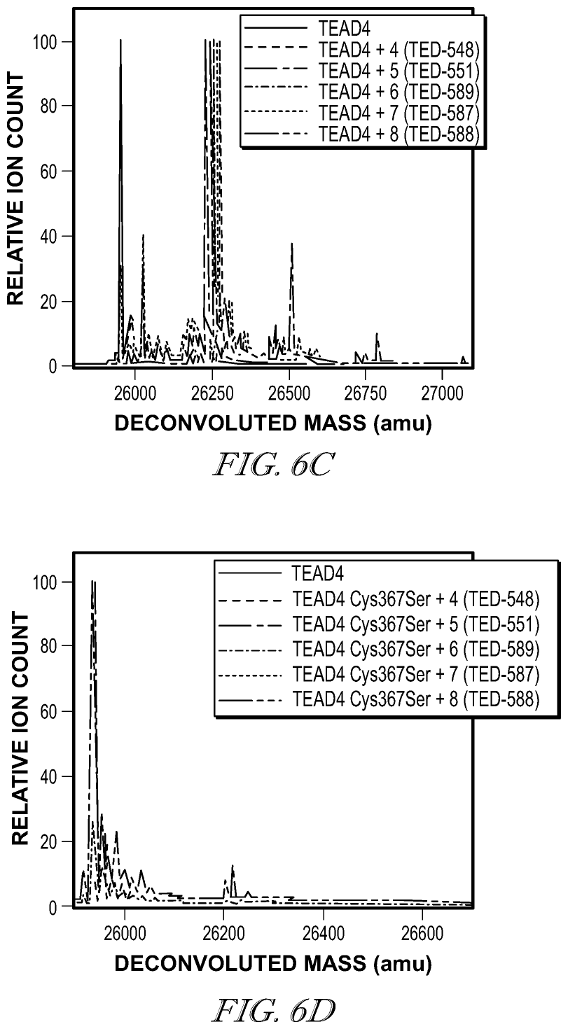

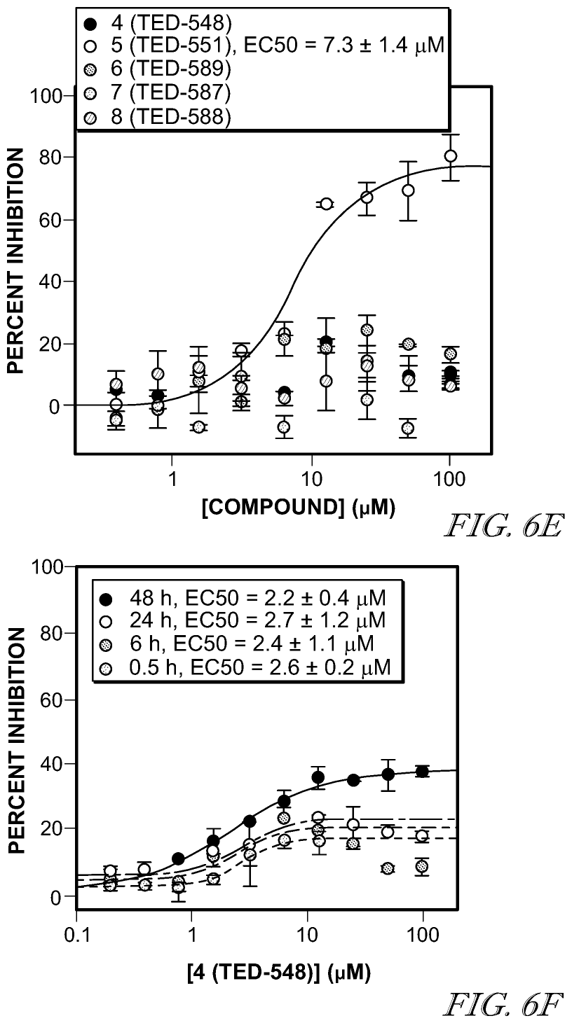

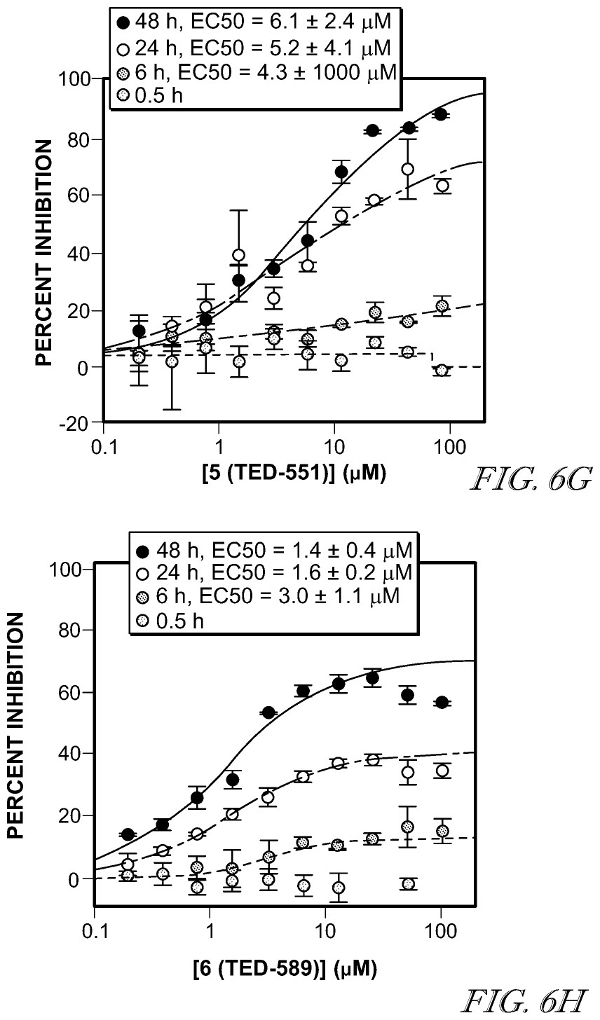

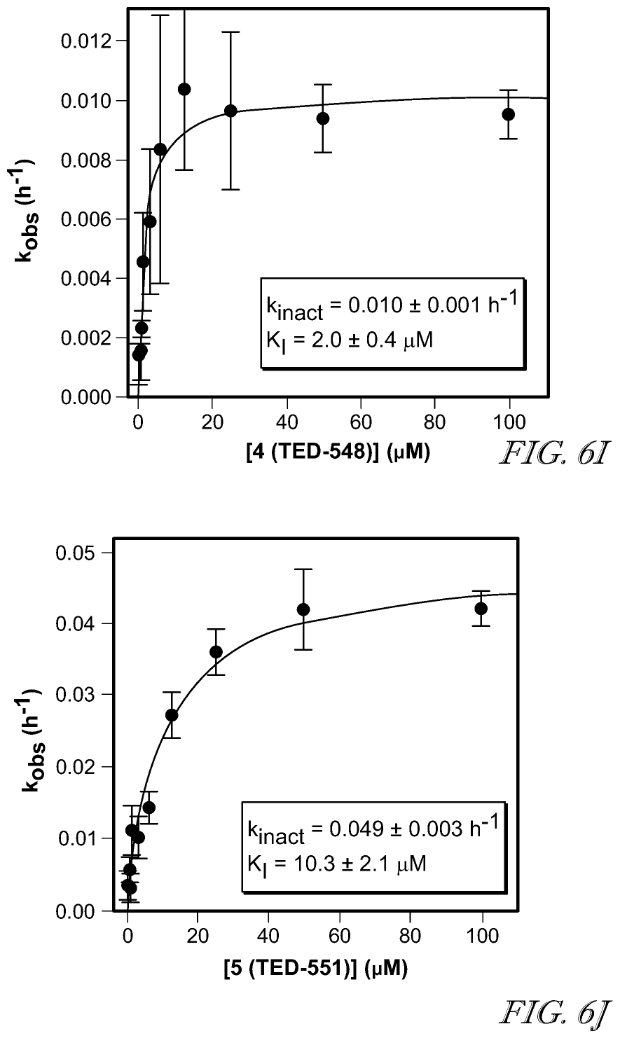

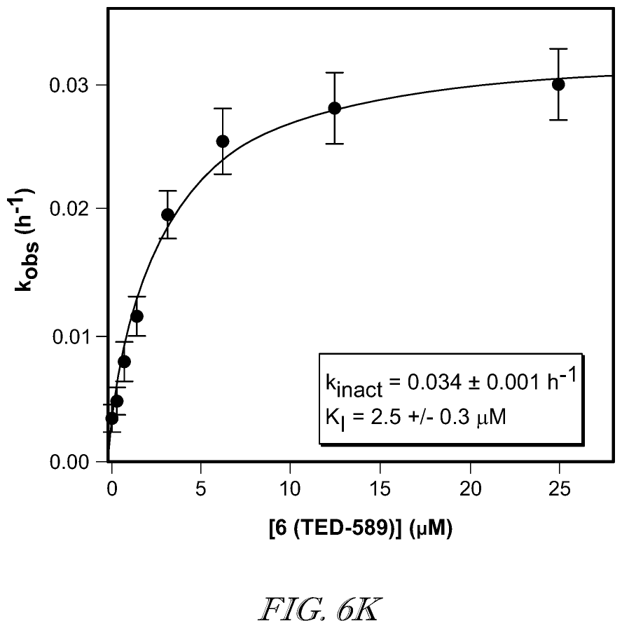

After 24 h incubation with TEAD4 at 4° C., compound 4, compound 5, and compound 6 showed a maximum inhibition of 31, 81, and 51% respectively, while compound 7 and compound 8 displayed less than 20% inhibition (FIG. 6A). Yet, the EC₅₀ of compound 4 was substantially lower (nearly an order of magnitude) than its parent compound 2, as well as the other derivatives. Compound 5 also inhibited the TEAD4 Cys367Ser mutant, in contrast to compound 4 and compound 6 (FIG. 6B). Compound 6 had improved EC₅₀ of 2.3±0.8 μM, while still being selective toward Cys-367. Whole protein mass spectrometry analysis of TEAD4 with the compounds showed that compound 4, compound 6, compound 7 and compound 8 formed single adducts, consuming all of the protein, while compound 5 formed more than one covalent complex (FIG. 6C). Furthermore, only compound 5 formed an adduct with the TEAD4 Cys367Ser mutant as evidenced by a minor peak corresponding to a mass of 26217 (FIG. 6D). The lack of TEAD4 inhibition by compound 7 and compound 8 (FIG. 6A), while still forming 100% adduct with TEAD4 (FIG. 6C), demonstrated that mere binding and reaction to Cys-367 on TEAD4 is not sufficient for inhibition of TEAD4 activity, as demonstrated with iodoacetamide (FIGS. 9A-9E). The five derivatives were also tested for inhibition of TEAD2 binding to Yap1. Compound 5 showed similar inhibition of TEAD2 and TEAD4 binding to Yap1, while compound 4 and compound 6 were much weaker inhibitors of TEAD2 compared to TEAD4 (FIG. 6E).

The three active derivatives showed concentration- and time-dependent inhibition of TEAD4 (FIG. 6F-H). The rate of inactivation of compound 4 is lower than its parent at 0.010±0.001 h-′, which resulted in a t_{1/2}^{∞} of 67.3 h (FIG. 6I). The rate of inactivation of compound 5 is slightly faster than its parent with a k_{inact} of 0.049±0.003 h⁻¹ corresponding to a half-life of 14.3 h (FIG. 6J). The rate of inactivation of compound 6, k_{inact}=0.034±0.003 h⁻¹ (t_{1/2}^{∞}=20 h) (FIG. 6K), was similar to parent compound 2.

FIG. 6A illustrates measured fluorescence polarization when TEAD4 was incubated with increasing concentration of compounds for 24 h at 4° C. followed by addition of FAM-Yap₆₀₋₉₉ (mean±s.d.; n=3). FIG. 6B illustrates FP measurement when TEAD4 Cys367Ser mutant was incubated with increasing concentrations of compounds for 24 h at 4° C. and then interacted with fluorescently labeled Yap peptide (mean±s.d.; n=3). FIG. 6C illustrates ESI mass spectrometry analysis after 10 μM TEAD4 was incubated with 200 μM compounds for 24 h at 4° C.

FIG. 6D illustrates ESI mass spectrometry analysis after 10 μM TEAD4 Cys367Ser mutant was incubated with 200 μM compounds for 24 h at 4° C. FIG. 6E illustrates FP measurement after His-TEAD2 was incubated with increasing concentration of compounds for 24 h at 4° C. followed by addition of FAM-Yap₆₀₋₉₉ (mean±s.d.; n=3). FIG. 6F illustrates FP measurement following time-dependent inhibition of TEAD4 by compound 4 at 10 different concentrations (0.1-100 μM) after 0.5, 6, 24 and 48 h incubations at 4° C. (mean±s.d.; n=3). FIG. 6G illustrates FP measurement following Time-dependent inhibition of TEAD4 by compound 5 at 10 different concentrations (0.1-100 μM) after 0.5, 6, 24 and 48 h incubations at 4° C. (mean±s.d.; n=3). FIG. 6H illustrates FP measurement following time-dependent inhibition of TEAD4 by compound 6 at 10 different concentrations (0.1-100 μM) after 0.5, 6, 24 and 48 h incubations at 4° C. (mean±s.d.; n=3). FIG. 6I illustrates FP measurement following time-dependent inhibition of TEAD4 by compound 4 at 10 different concentrations (0.1-100 μM) after 0.5, 6, 24, and 48 h incubations at 4° C. The observed rate of inactivation (k_{obs}) was calculated for each concentration of compound and is plotted against the concentration of compound (mean±s.d.; n=3). FIG. 6J illustrates FP measurement following time-dependent inhibition of TEAD4 by compound 5 at 10 different concentrations (0.1-100 μM) after 0.5, 6, 24, and 48 h incubations at 4° C. The observed rate of inactivation (k_{obs}) was calculated for each concentration of compound and is plotted against the concentration of compound (mean±s.d.; n=3). FIG. 6K illustrates FP measurement following time-dependent inhibition of TEAD4 by compound 6 at 10 different concentrations (0.1-100 μM) after 0.5, 6, 24, and 48 h incubations at 4° C. The observed rate of inactivation (k_{obs}) was calculated for each concentration of compound and is plotted against the concentration of compound (mean±s.d.; n=3).

Cell Culture

HEK-293 and GBM43 cells were cultured in DMEM medium with glutamine (Cellgro, Manassas, Va.) supplemented with 10% FBS and 1% penicillin/streptomycin in 5% CO₂ at 37° C.

Luciferase Reporter Assay

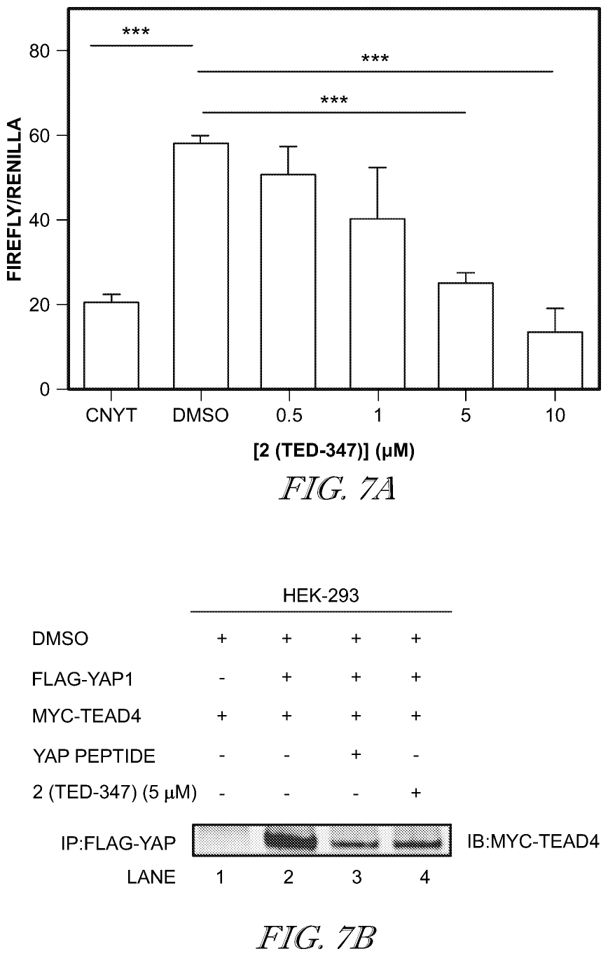

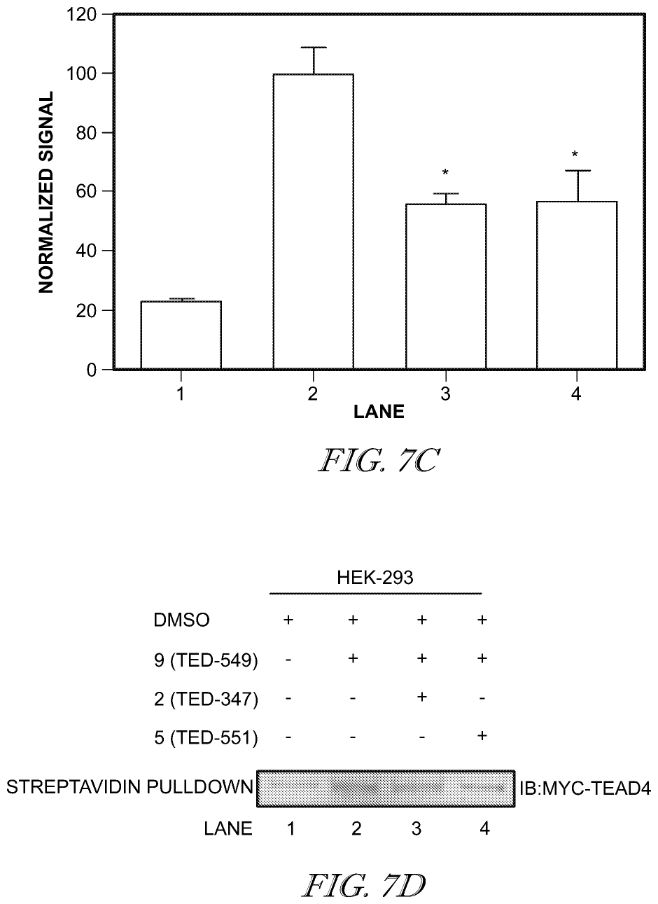

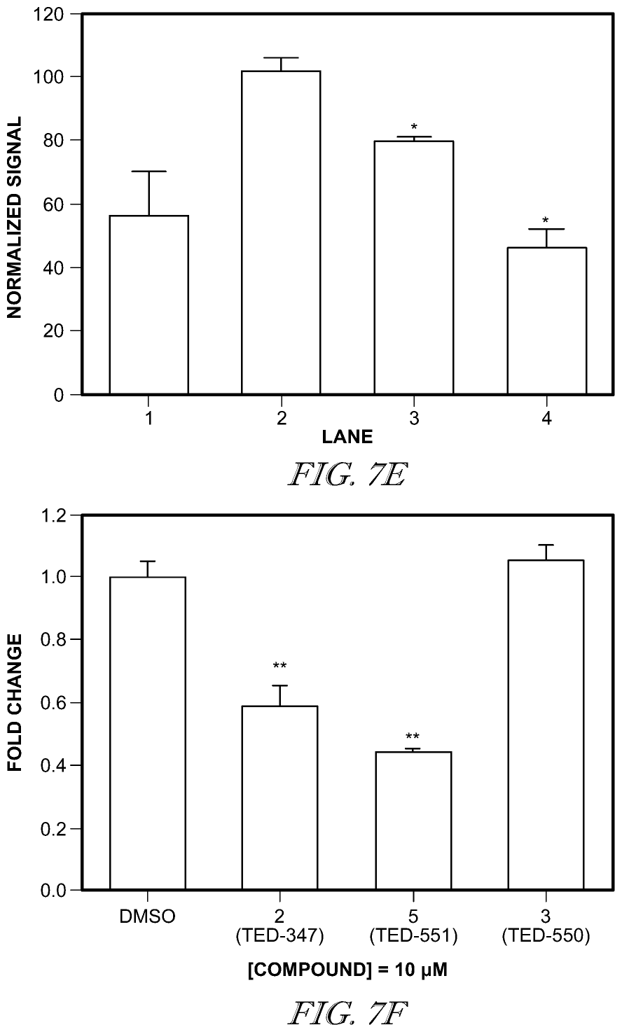

HEK-293 cells plated at 2.4×10⁴ cells/well in a 96-well microplate were transfected after 24 hours with the a pGL3.1 reporter containing the CTGF promoter and a plasmid encoding TK-Renilla luciferase in combination with control vectors or vectors that express Yap1 and TEAD4. After 48 h cells, were treated with 0.5, 1.0, 5.0 or 10 μM of compound 2 for another 48 h. Luciferase activity was measured according to the Dual-Glo luciferase assay (Promega) instructions using a Biotek Synergy Neo2 plate reader. Relative luciferase activity represents the ratio of firefly/renilla luminescence values. See FIG. 7A.

Covalent Pull Down of TEAD4

HEK293 cells transfected with the myc-TEAD4 construct were grown for 48 h and then treated with DMSO or with 25 μM of compound 2 for an additional 48 h. Cells were then harvested in lysis buffer (50 mM Tris-HCl, pH 7.3, 150 mM NaCl, 0.5 mM EDTA, 1% Triton X-100, PhosSTOP phosphatase inhibitor cocktail, and EDTA-free protease inhibitors cocktail). Cell lysates containing 2 mg of protein were incubated with the indicated compounds or DMSO for 24 h. Extracts were then incubated with Dynabeads M-280 Streptavidin (Sigma-Aldrich) for 2 h at 4° C. Dynabeads were then washed and bound proteins were denatured and eluted according to the manufacturer's instructions. Relative levels of myc-TEAD4 from each complex were measured by immunoblot analysis with the anti-c-Myc antibody (1:5,000, Sigma-Aldrich).

Co-Immunoprecipitation (Co-IP)

HEK293 cells transfected with Flag-YAP1 alone or in combination with myc-TEAD4 were incubated with DMSO or the indicated amount of compounds for 48 hours. Cells were harvested in lysis buffer (50 mM Tris-HCl, pH 7.3, 150 mM NaCl, 0.5 mM EDTA, 1% Triton X-100, PhosSTOP phosphatase inhibitor cocktail, and complete EDTA-free protease inhibitors cocktail). Extracts were immunoprecipitated with magnetic beads coupled to the M2 (anti-Flag) antibody (Sigma-Aldrich) for 4 h at 4° C. Dynabeads were then washed and bound proteins were denatured and eluted according to the manufacturer's instructions. Relative levels of myc-TEAD4 from each complex was then measured by immunoblot analysis with the anti-c-Myc antibody (1:5,000, Sigma-Aldrich). See FIGS. 7 B and 7C.

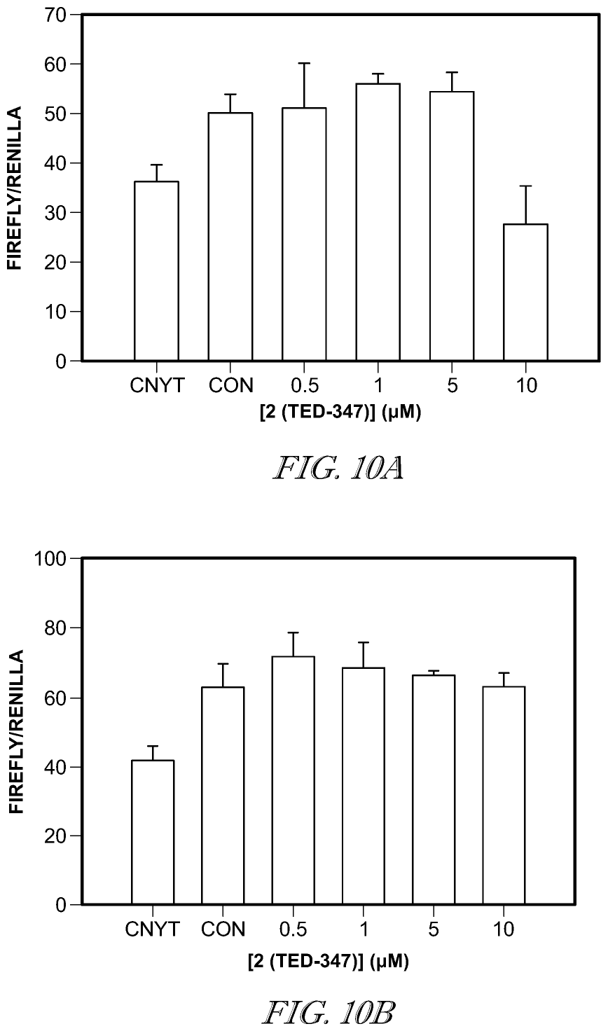

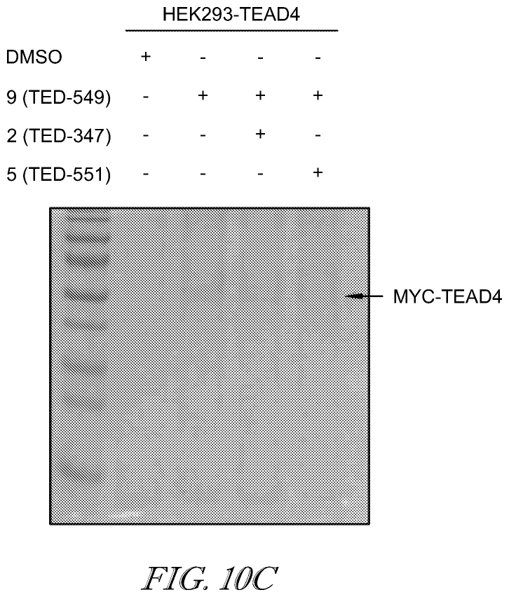

The effect of compound 2 on the intracellular transcriptional activity of TEAD4 was compared to its effects on the interaction of TEAD4 with Yap (FIG. 7A-7F). Treatment of cells transfected with a TEAD reporter over 48 h with compound 2 at 5 μM resulted in over 70% reduction in reporter activity, whereas cells treated with 10 μM of compound 2 showed a complete loss of reporter activity. Less activity is observed at 24 h suggesting time-dependent activity in cells (FIG. 10A). To further establish the selectivity of the small molecule, the TEAD4 transcriptional activity luciferase reporter assays were repeated using instead transfected Cys367Ala mutant. It was found that treatment of HEK-293 cells with compound 2 did not result in the inhibition of TEAD4 transcriptional activity as was observed for wild-type TEAD4 (FIG. 10B). Consistent with these effects being a result of disruption of the TEAD4.Yap1 interaction, cells incubated with 5 μM of compound 2 showed a significant loss of co-immunoprecipitation of Myc-tagged TEAD4 with Flag-Tagged Yap1 (FIGS. 7A-C). To establish that compound 2 forms a covalent adduct with TEAD4 in cells, a biotin-conjugated variant termed compound 9 was synthesized. Following addition of compound 9 to cell lysates, TEAD4 was specifically detected by immunoblot analysis in a streptavidin pull-down, consistent with compound 2 directly engaging TEAD4 in cells in a covalent complex (FIG. 7D, 7E, FIG. 10B). The reduction in TEAD4 in compound 9 containing samples that were also treated with higher concentrations of compound 2 or compound 5 indicates that these compounds compete with compound 9 for binding to TEAD4 (FIGS. 7D and 7E lanes 3 and 4). FIGS. 10A-10C shows that compound 2 does not inhibit TEAD mutant transcriptional activity and protein-protein interactions in cell culture. (A) The activity of the TEAD4 luciferase reporter was measured in HEK-293 cells at 24 h treated with either vehicle or compound 2 (TED-347); mean±s.d.; n=3 replicates. (B) The activity of the TEAD4 luciferase reporter was measured in HEK-293 cells treated with either vehicle or compound 2 (TED-347); mean±s.d.; n=2 replicates. (C) Coomassie-stained gel of the pull-down sample. The pull-down shows little background suggesting that the compound is likely selective to TEAD4.

RNA Extraction and Real-Time PCR

HEK293 cells co-transfected the Flag-YAP and myc-TEAD4 constructs were incubated with DMSO or the indicated amount of compounds for 48 h. See FIGS. 7A and 7B. Total RNA was purified using the RNeasy plus mini kit (QIAGEN, Hilden, Germany) according to the manufacturer's instructions. Complementary DNA was synthesized from 500 ng total RNA with Oligo-dT primers and the Multi-Scribe reverse transcriptase (Fisher, Waltham, Mass.) according to the manufacturer's instructions.

Real-time PCR reactions utilized 100 ng cDNA, 200 nM gene specific primers and the Sensifast No-ROX mix (Bioline, Taunton, Mass.) in a total volume of 20 μl. All measurements were carried out in triplicate using an Eppendorf Mastercycler® RealPlex2. The sequences of primers for CTGF were forward, 5′-TTGGCCCAGACCCAACTA-3 (SEQ ID NO: 1); and reverse, 5′-GCAGGAGGCGTTGTCATT-3′ (SEQ ID. NO: 2). The primer sequences for (3-actin were forward, 5′-TTGGCAATGAGCGGTTCC-3 (SEQ ID NO: 3); and reverse, 5′-GTTGAAGGTAGTTTCGTGGATG-3′ (SEQ ID NO: 4).

To monitor endogenous TEAD activity, the levels of CTGF transcript were measured by qRT-PCR from control cells and cells incubated with compound 2 or compound 5. Cells incubated with compound 2 and compound 5 showed a significant reduction in CTGF transcript levels versus control cells (FIG. 7F). Cells incubated with compound 3, which lacks the reactive moiety necessary to form an adduct with TEAD4, showed similar levels of CTGF transcript versus control cells.

Sphere-Forming Assay

Early-passage GBM43 cells were cultured in ultralow adherence plates (Corning Inc.) at 1 to 2×10³ cells/mL in DMEM supplemented with 1% nitrogen (Invitrogen), 2% B27 (Invitrogen), 25 ng/mL epidermal growth factor (EGF, 25 ng/mL fibroblast growth factor (FGF) (R&D Systems, Inc.), 2 ng/mL platelet growth factor (GFG) (R&D Systems Inc.), 2 ng/mL platelet growth factor, and 100 ng/mL each of penicillin and streptomycin (Invitrogen Inc.). Individual cells were recovered from spheres by incubation with Accutase (Sigma-Aldrich) for 10-15 minutes. Cells (50-100/well) were then seeded in fresh sphere-forming media in 96-well plates. After 2-3 days, Neurospheres containing 6 to 8 cells were treated with compounds 1, 2, and 5.

HEK-293 were cultured in DMEM with glutamine (Cellgro, Manassas, Va.) supplemented with 10% FBS and 1% penicillin/streptomycin in a 5% CO₂ atmosphere at 37° C. Tumors were expanded by passage in the flank of NOD/SCIDγnull mice. To generate GBM43 cell lines, tumors were harvested, disaggregated, and maintained in 2.5% FBS for 14 days on Matrigel-coated plates (BD Biosciences) to remove murine fibroblasts. In-vitro GBM43 cell lines were propagated in DMEM with 10% FBS for no more than 7 passages. Cell line identity was confirmed by DNA fingerprint analysis (IDEXX BioResearch) for species and baseline short-tandem repeat analysis testing. GBM43 spheroids were generated by plating early-passage cells at 2.5×10⁴ cells per well in 96-well ultralow attachment plates (Corning Inc.) in DMEM/F12 (1:1; GIBCO) supplemented with 2% B27 supplement (GIBCO), 20 ng/mL epidermal growth factor (EGF), and 20 ng/mL fibroblast growth factor (FGF) (Peprotech) for 2 days. The spheroids were then treated with compounds 1, 2, and 5 and growth analyzed by Alamar blue staining.

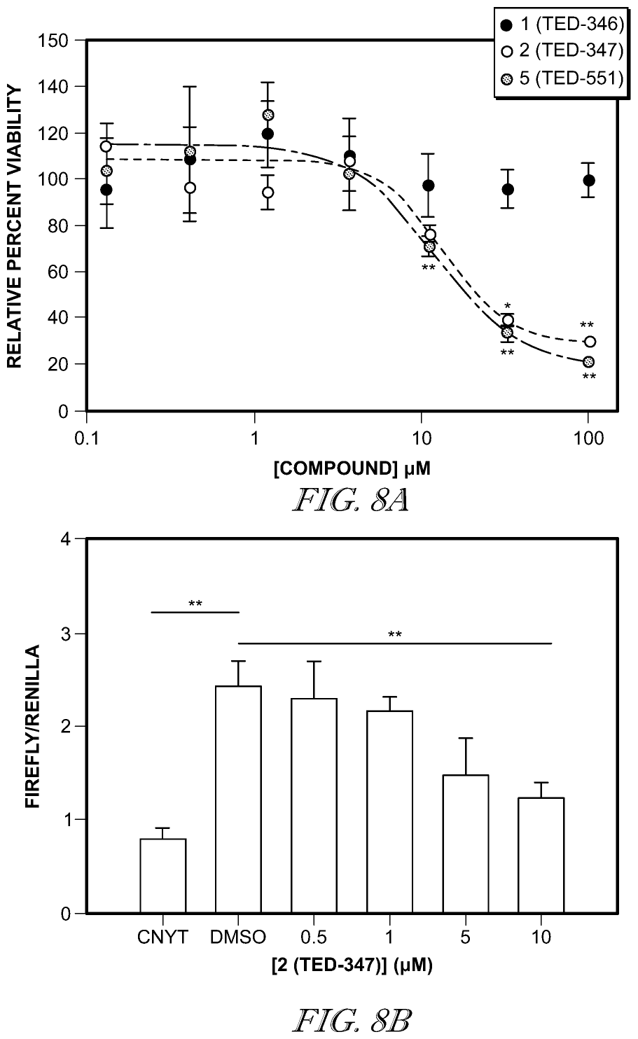

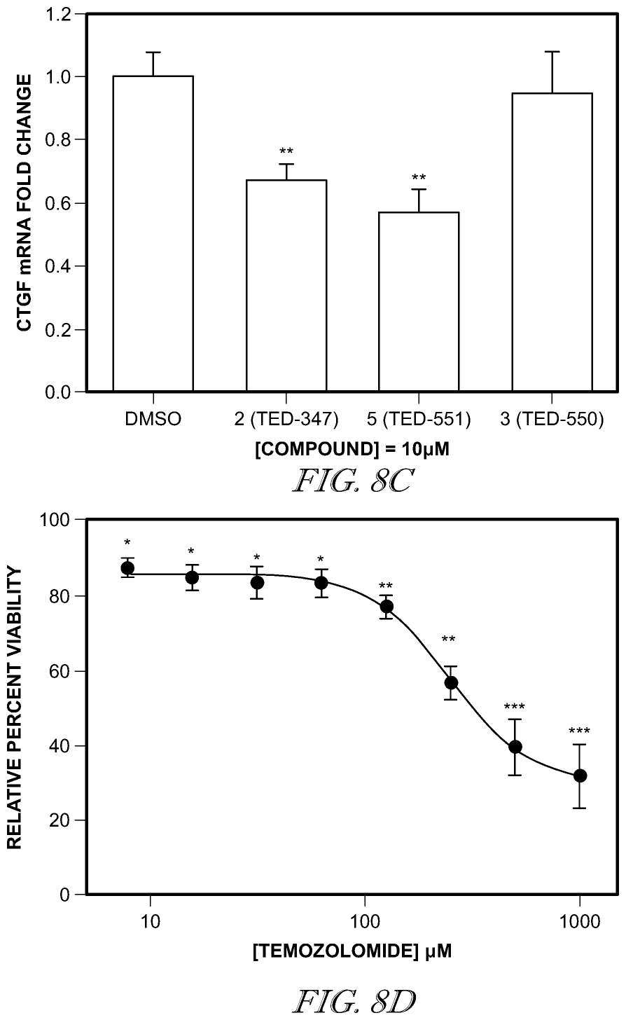

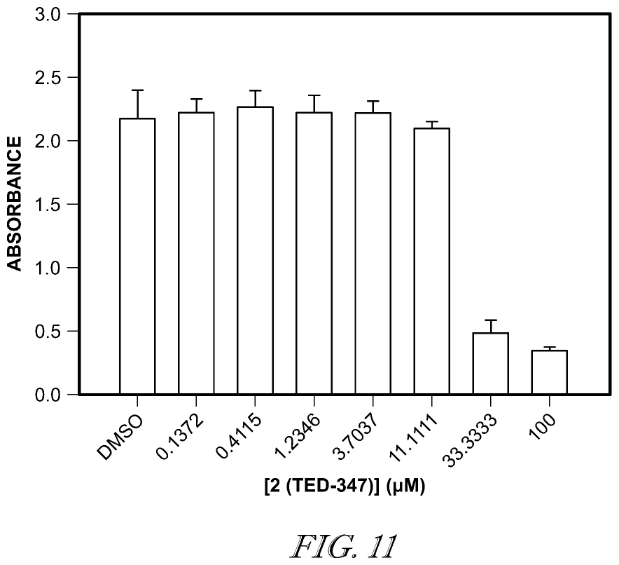

Hippo signaling promotes tumor growth and invasion in a range of cancers including GBM. We investigated the effects of compounds 1, 2, and 5 on GBM cell viability using patient-derived GBM43 cells that were grown as three-dimensional spheroids (FIG. 8A). Both compound 2 and compound 5 inhibited GBM43 cancer cell viability. At 10 μM, which is the concentration that was used to demonstrate inhibition of TEAD4 activity in cells, the compounds inhibit GBM43 cell viability by 30%. At this concentration, the compound did not show any effect on cell viability of non-transformed normal astrocytes (FIG. 11). Compound 1, which does not inhibit TEAD4.Yap1, did not affect GBM43 cancer cell growth (FIG. 8A). Compound 2 also inhibited TEAD4 transcriptional activity in GBM43 cells (FIG. 8B) in a concentration-dependent manner. Similarly, as shown in FIG. 8C, both compound 2 and compound 5 suppressed CTGF transcript levels while compound 1 had no effect versus cells treated with vehicle. The potency of compounds 2 and 6 were compared to temozolomide, which is the standard of care for patients with glioblastoma. Temozolomide inhibited GBM43 spheroid growth with a substantially higher EC₅₀ of 244±24 μM (FIG. 8D). FIG. 11 illustrates the following: human astrocytes cells were plated 24 h before treatment in collagen I coated 96-well plates in Dulbecco's modified Eagle's medium, supplemented with 20 μg/mL insulin, 5 μg/mL N-acetylcysteine, 10% fetal bovine serum, 10 μM hydrocortisone and antibiotics; then treated with DMSO or TED-347 at indicated concentrations for 72 h at 37° C. and 5% CO₂ in a humidified incubator. Stock MTS (Promega) solution (20 μL per 100 μL medium) was added to all wells of an assay, and plates were incubated at 37° C. for 4 h, and read on a BioTek Synergy 2 reader, using a test wavelength of 490 nm; mean±s.d.; n=2 replicates.

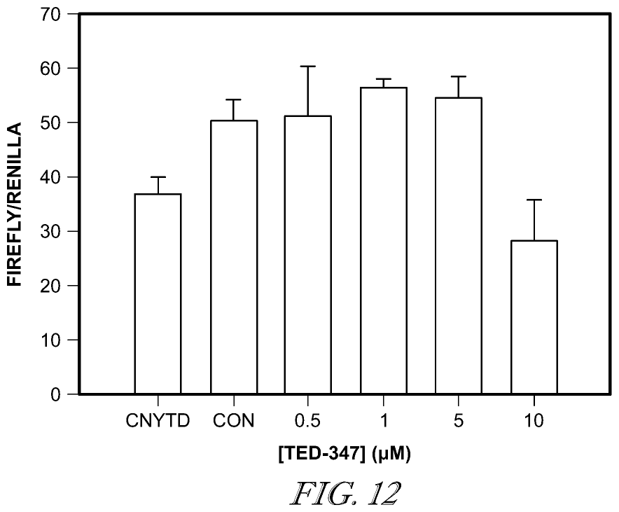

FIG. 12 illustrates that compound 2 inhibits TEAD transcriptional activity in a concentration-dependent manner in cell culture. The activity of the TEAD4 luciferase reporter was measured in HEK-293 cells at 24 h treated with either vehicle or compound 2; mean±s.d.; n=3 replicates.

FIG. 8A illustrates relative percent viability of spheroids of patient-derived GBM43 glioblastoma cell lines were grown and treated with compound 1, compound 2, and compound 5; mean±s.d.; n=3 biological replicates. FIG. 8B illustrates the measured activity of the TEAD4 luciferase reporter in GBM43 cells treated with either vehicle or compound 2. CNYT corresponds to no transfection; mean±s.d.; n=3 biological replicates. FIG. 8C illustrates qRT-PCR analysis of CTGF levels following treatment of HEK-293T cells with compounds; mean±s.d.; n=3 biological replicates. FIG. 8D illustrates relative percent viability of spheroids of patient-derived GBM43 glioblastoma cell lines were grown and treated with temozolomide; mean±s.d. of biological replicates (n=3). P values were calculated using two-tailed t-tests. *P<0.05, **P<0.005, ***P<0.0005.

Other variations or embodiments will be apparent to a person of ordinary skill in the art from the above-description. Thus, the foregoing embodiments are not to be construed as limiting the scope of the claimed invention. All references disclosed are expressly incorporated by reference in in their entirety.

1. (canceled)

2. (canceled)

3. A compound of Formula (1b) 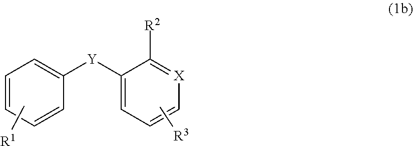
wherein
Y is selected from the group consisting of —C(O)—, —NH—, —O—, —S—, —CH₂—, and —S(O)₂;
R¹ is selected from the group consisting of —C(R)₃, thiophene, —O(CH₂)ₙ(OCH₂CH₂)ₘNH— biotin, and lower (C₁-C₆) alkyl, where one or more of the carbon atoms in lower (C₁-C₆) are replaced by oxygen, n is an integer from 1 to 6 and m is an integer from 1 to 3; and where R is a halogen;
R² is selected from the group consisting of —CH₂CN, —CN, —NHC(O)CHCH₂, —S(O)₂CHCH₂, —NHC(O)CHCHCH₂N(CH₃)₂, —NHC(O)CCH, —C(O)CH₂R^{2′}, and —NHC(O)CH₂R^{2′}, where R^{2′} is halogen or lower (C₁-C₆) alkyl, where one or more the hydrogens in lower (C₁-C₆) alkyl are replaced with a halogen;
R³ is H or C₁-C₆ alkoxy,
X is carbon or a heteroatom independently selected from nitrogen, oxygen and sulfur;
or a pharmaceutically acceptable salt thereof.

4. The compound or pharmaceutically acceptable salt thereof of claim 3, wherein lower (C₁-C₆) alkyl is methyl, ethyl, or n-propyl.

5. The compound or pharmaceutically acceptable salt thereof of claim 3, wherein R^{2′} is a halogen.

6. The compound or pharmaceutically acceptable salt thereof claim 3, wherein R^{2′} is chloro.

7. The compound or pharmaceutically acceptable salt thereof of claim 3, wherein R¹ is C(R)₃ where R is a halogen.

8. The compound or pharmaceutically acceptable salt thereof of claim 3, wherein X is carbon or nitrogen.

9. The compound or pharmaceutically acceptable salt thereof of claim 3, wherein X is carbon.

10. The compound or pharmaceutically acceptable salt thereof of claim 3, wherein X is nitrogen.

11. The compound or pharmaceutically acceptable salt thereof of claim 3, wherein Y is —NH—.

12. The compound or pharmaceutically acceptable salt thereof of claim 3, wherein R² is —C(O)CH₂R^{2′}.

13. The compound or pharmaceutically acceptable salt thereof of claim 12, wherein R^{2′} is a halogen.

14. The compound or pharmaceutically acceptable salt thereof of claim 12, wherein R^{2′} is chloro.

15. The compound or pharmaceutically acceptable salt thereof of claim 3, wherein R³ is a H.

16. The compound or pharmaceutically acceptable salt thereof of claim 3, wherein R³ is a alkoxy.

17. The compound or pharmaceutically acceptable salt thereof of claim 3, wherein R³ is methoxy.

18. The compound or pharmaceutically acceptable salt thereof of claim 3, wherein R¹ is thiophene.

19. The compound or pharmaceutically acceptable salt thereof of claim 3, wherein R¹ is —O(CH₂)ₙ(OCH₂CH₂)ₘNH-biotin.

20.-22. (canceled)

23. A compound selected from the group consisting of 2-chloro-1-(2-((3-(trifluoromethyl)phenyl) amino)phenyl)ethanone, 2-chloro-1-(2-((3-(2-methoxyethoxy)phenyl)amino)phenyl)ethanone, 2-chloro-1-(3-((3-(trifluoromethyl)phenyl)amino)pyridin-2-yl)ethanone, 2-chloro-1-(4-methoxy-2-((3-(trifluoromethyl)phenyl)amino)phenyl)ethanone, 2-chloro-1-(2-((4-(thiophen-2-yl)phenyl)amino)phenyl)ethanone, 2-chloro-1-(2-((3-(thiophen-2-yl)phenyl)amino)phenyl)ethanone, N-(2-(2-((5-(3-((2-(2-chloroacetyl)phenyl)amino)phenoxy)pentyl)oxy)ethoxy)ethyl)-5-(2-oxohexahydro-1H-thieno[3,4-d]imidazol-4-yl)pentanamide, 2-fluoro-1-(2-((3-(trifluoromethyl)phenyl)amino)phenyl) ethanone, 2-chloro-N-(2-((3-(trifluoromethyl)phenyl)amino)phenyl)acetamide, and N-(2-((3-(trifluoromethyl)phenyl)amino)phenylacrylamide; or a pharmaceutically acceptable salt thereof.

24.-26. (canceled)

27. A method to treat cancer in a mammal in need thereof, comprising administering an effective amount of a compound Formula (1b) 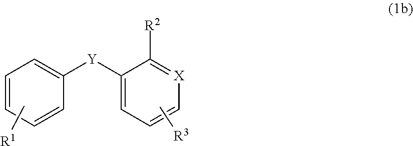
wherein
Y is selected from the group consisting of —C(O)—, —NH—, —O—, —S—, —CH₂—, and —S(O)₂;
R¹ is selected from the group consisting of —C(R)₃, thiophene, —O(CH₂)ₙ(OCH₂CH₂)ₘNH— biotin, and lower (C₁-C₆) alkyl, where one or more of the carbon atoms in lower (C₁-C₆) are replaced by oxygen, n is an integer from 1 to 6 and m is an integer from 1 to 3; and where R is a halogen.
R² is selected from the group consisting of —CH₂CN, —CN, —NHC(O)CHCH₂, —S(O)₂CHCH₂, —NHC(O)CHCHCH₂N(CH₃)₂, —NHC(O)CCH, —C(O)CH₂R^{2′}, and —NHC(O)CH₂R^{2′}, where R^{2′} is halogen or lower (C₁-C₆) alkyl, where one or more the hydrogens in lower (C₁-C₆) alkyl are replaced with a halogen;
R³ is H or C₁-C₆ alkoxy,
X is carbon or a heteroatom independently selected from nitrogen, oxygen and sulfur;
or a pharmaceutically acceptable salt thereof.

28.-48. (canceled)

49. The method of claim 27, wherein the cancer is a solid tumor, lung cancer, thyroid cancer, skin cancer, ovarian cancer, colon cancer, rectal cancer, prostate cancer, pancreatic cancer, esphogal cancer, liver cancer, or breast cancer.

50.-57. (canceled)


SEQUENCE LISTING (4 sequences)

SEQ ID NO: 1 - DNA, 18 bp, Artificial Sequence
  CTGF forward primer
gcaggaggcgttgtcatt

SEQ ID NO: 2 - DNA, 18 bp, Artificial Sequence
  CTGF reverse primer
gcaggaggcgttgtcatt

SEQ ID NO: 3 - DNA, 18 bp, Artificial Sequence
  beta-actin forward primer
ttggcaatgagcggttcc

SEQ ID NO: 4 - DNA, 22 bp, Artificial Sequence
  beta-lactin reverse primer
gttgaaggtagtttcgtggatg